(19) United States
(12) Patent Application Publication
(10) Pub. No.: US 2025/0164357 A1
(43) Pub. Date: May 22, 2025

(54) ISOLATING EXTRACELLULAR MATRIX BODIES

(71) Applicant: Aufbau Medical Innovations Ltd., Dublin (IE)

(72) Inventors: John T. G. PENA, New York, NY (US); John IRWIN, North Brunswick Township, NJ (US); Farideh MEHRAEIN-GHOMI, Highland Park, NJ (US); James Murray MITCHELL, New York, NY (US); Harmon Lawrence REMMEL, New York, NY (US)

(21) Appl. No.: 18/929,328

(22) Filed: Oct. 28, 2024

Related U.S. Application Data
(63) Continuation of application No. PCT/US2023/020288, filed on Apr. 27, 2023.
(60) Provisional application No. 63/335,890, filed on Apr. 28, 2022.

(51) Int. Cl. G01N 1/30; G01N 1/40; G01N 33/50

(52) U.S. Cl. CPC G01N 1/30; G01N 1/4077; G01N 33/50; G01N 2001/305; G01N 2001/4088; G01N 2800/60

(57) ABSTRACT

This invention relates to methods for separating, isolating and/or enriching extracellular matrix bodies in a biological fluid. More particularly, this invention discloses methods for isolating and detecting extracellular matrix bodies from a biological sample as medical information and/or for use in diagnosis, prognosis and monitoring disease.

### CROSS-REFERENCE TO RELATED APPLICATIONS

This application is a continuation of International Patent Application No. PCT/US2023/020288, filed Apr. 27, 2023, which claims priority to U.S. Provisional Patent Application No. 63/335,890, filed Apr. 28, 2022, the entire contents of which are incorporated herein by reference.

### TECHNICAL FIELD

This invention relates to methods for isolating, separating and/or enriching extracellular matrix bodies from biological fluids. More particularly, this invention discloses methods for isolating, detecting and measuring extracellular matrix bodies from a biological fluid as medical or diagnostic information. The methods include centrifugation and/or filtration of a biological fluid.

### BACKGROUND

Conventional methods for acquiring diagnostic information from biological samples are limited by the ability to detect features associated with disease. To detect the disease, the source of the biological sample is chosen to be closely connected to disease pathology. For example, intact tissue samples studied in relation to disease can be excised from a subject which ensures that the sample is correlated to the disease. Drawbacks of such conventional methods include the need for invasive biopsy for sampling the disease pathology.

Improvements in conventional methods for obtaining diagnostic information from biological samples include studying cells, exosomes or other isolated structures. However, these methods have major limitations when those well-known structures do not readily reflect the disease pathology. This information can have more tenuous connection to the disease and require significant assumptions underlying any diagnostic analysis.

Moreover, conventional methods for separation and analysis of biological fluids do not in general detect all components of the fluid. Most of the fluid and its components are lost, discarded or ignored in conventional methods or assays for nucleic acids or proteins, so that precious material is unused and valuable information is lost.

In addition, specific components of biological fluids are often hidden or masked amongst the many other components, so that any information they reflect is completely missed by conventional methods.

Further, additional components of biological fluids have generally not been exploited in medicine and pharmaceutics. Conventional methods have failed to perceive, appreciate, or examine significant components of biological fluids, in part because their presence is overwhelmed by other features, such as cells, cell components, and/or cell debris. Consequently, conventional methods intentionally or inadvertently discard components obscured by cells in biological fluids. However, such additional significant components of biological fluids can provide a wealth of medical information.

Additional drawbacks of conventional methods include the use of biomarkers which are inherently limited by remote association with disease pathology and significant uncertainties of accurate measurement.

What is needed are methods and compositions for separating, isolating, and/or enriching components from bodily fluids for use as diagnostic information. Methods for isolating and measuring components from a biological sample are needed for improving and increasing accuracy of diagnostic analysis relevant to a particular biology and/or disease.

There is an urgent need for methods and compositions for reducing the need for invasive biopsy, which can provide fractions of a biological material having strong connection to disease pathology. Methods for preparing and isolating samples from various biological fluids are needed to expand the range of diagnostic information available toward particular pathologies and disease.

### BRIEF SUMMARY

This invention encompasses methods for separating and isolating biological samples to obtain extracellular components of a biological fluid which are novel features reflecting diagnostic information.

The novel biological components can be associated with a disease and/or closely connected to a disease pathology. More particularly, isolated biological components from biological fluids can contain substances which inform of a disease and advantageously reduce the need for invasive biopsy for sampling the disease pathology.

Disclosures of this invention include methods for preparing samples for obtaining diagnostic information from biological samples. The methods include separating, enriching and/or isolating structures derived ultimately from an extracellular matrix region. The structures can directly reflect components of disease pathology extant in the isolates. Isolated structures may provide biomarker information with a direct connection to the disease and being useful in diagnostic evaluation and analysis. Methods disclosed herein include biomarker information with significantly enhanced level of detection and/or measurement.

Embodiments of this invention include methods and compositions for separating, isolating, and/or enriching extracellular matrix bodies from bodily fluids for diagnostic and/or therapeutic use. The use of extracellular matrix bodies isolated and/or enriched in a biological sample can surprisingly improve diagnostic analysis for a particular biological condition or disease.

Methods and compositions of this disclosure can advantageously reduce the need for invasive patient biopsy by using extracellular matrix bodies which are isolated from bodily fluid samples.

This invention includes methods for preparing and isolating samples from various biological fluids which surprisingly expands the range of diagnostic information available toward particular pathologies and disease. The fractions obtained from a biological material can have strong connection to disease pathology.

This invention can separate, isolate or enrich hidden components including extracellular matrix bodies from biological fluids and unlock their potential for medical information.

Embodiments of this disclosure contemplate methods for preparing samples for medical information, such as for diagnosis, prognosis or monitoring of disease in a subject, by isolating extracellular matrix bodies from a biological sample. In some embodiments, the extracellular matrix bodies can be directly associated with the disease.

Additional embodiments of this invention can isolate, extract, and/or utilize extracellular matrix bodies that are a source of multiple and specific biomarkers.

In certain aspects, extracellular matrix bodies can operate as biomarkers through their morphological features. In further aspects, extracellular matrix bodies can operate by containing isolated biochemical markers that may be presented in a disease pathway.

Embodiments of this invention include the following:

A process for separating, isolating or enriching extracellular matrix bodies in a biological fluid, the process comprising centrifuging or filtering the biological fluid. The process may comprise
removing cells or cell debris by centrifuging the biological fluid; and
separating, isolating or enriching extracellular matrix bodies from the biological fluid by centrifuging or filtering the biological fluid. The removing cells or cell debris by centrifuging the biological fluid may comprise low speed centrifugation. The removing cells or cell debris by centrifuging the biological fluid may comprise centrifugation which applies about 200 to 5,000 g for 1-10 minutes, or 100 to 1,200 g for 1-10 minutes. The separating, isolating or enriching extracellular matrix bodies from the biological fluid by centrifuging may create a pellet by applying forces for times and speeds below levels needed to force small particles into the pellet, so that the pellet is substantially free of particles smaller than about 1 micrometer. The separating, isolating or enriching extracellular matrix bodies from the biological fluid by centrifuging may create a pellet by applying 3,000-20,000 g for 1-100 minutes, or 6,000-100,000 g for 1-200 minutes. The separating, isolating or enriching extracellular matrix bodies from the biological fluid by centrifuging may create a pellet by applying 3,000-20,000 g for 1-100 minutes, or 6,000-100,000 g for 1-200 minutes, and re-suspending the pellet. The separating, isolating or enriching extracellular matrix bodies from the biological fluid by filtering can be performed with a filter for passing particles of less than a cutoff size, wherein the cutoff size is 1, or 2, or 3, or 4, or 5, or 6, or 10 micrometers, and the extracellular matrix bodies are extracted from the filter and re-suspended.

When the biological fluid is centrifuged, it may preferably be centrifuged by differential centrifugation or serial centrifugation. When differential centrifugation is performed, it may preferably be done at from about 4,000 to about 15,000 g, or more preferably at from about 6,000 to about 12,000 g.

When a biological fluid is filtered, it may preferably be filtered by single pass membrane filtration or multiple pass serial membrane filtration. For example, a cellulose acetate membrane filter may be used with a pore size of preferably from about 0.2 to about 1 micron, or more preferably from about 0.45 to about 2 microns.

The process for separating, isolating or enriching extracellular matrix bodies in a biological fluid may comprise both centrifuging and filtering the biological fluid.

The process above, comprising additional separating, isolating or enriching extracellular matrix bodies from a supernatant after centrifuging the biological fluid, or from a retentate, precipitate, residue, or extract after filtering the biological fluid, or from a filtrate after filtering the biological fluid, wherein the additional separating, isolating or enriching is done by microfluidic device, centrifugation, chromatography, chemical precipitation, filtration, immuno- or affinity-capture, electrophoresis, AC electrokinetics, or a combination thereof.

The process above, wherein the separating, isolating or enriching captures at least a majority of the extracellular matrix bodies from the biological fluid. The process above, wherein the separating, isolating or enriching captures at least a majority of the extracellular matrix bodies from the biological fluid with an absence of cells. The process above, wherein the separating, isolating or enriching captures substantially all of the extracellular matrix bodies from the biological fluid. The process above, wherein the separating, isolating or enriching captures substantially all of the extracellular matrix bodies from the biological fluid with an absence of cells. The process above, wherein the level of separated, isolated or enriched extracellular matrix bodies is a biomarker for medical, diagnostic or prognostic information. The process above, wherein the separated, isolated or enriched extracellular matrix bodies comprise biomarkers in the form of a protein, a polypeptide, a lipid molecule, a lipoparticle, a carbohydrate, a nucleic acid molecule, or an expression level of a nucleic acid. The process above, wherein the separated, isolated or enriched extracellular matrix bodies comprise biomarkers in the form of extracellular proteins, RNA, DNA, or a protein in Table 1.

For example, a biomarker can be the quantity of one or more of, for example, 1, 2, 3 or 4 of the proteins set out in Table 1 below. In another example, the biomarker may be the quantity of fibronectin.

This invention includes processes for separating, isolating or enriching extracellular matrix bodies in a biological fluid, the process comprising
applying an initial centrifugation step to the biological fluid for removing cells or cell debris; and
applying one or more serial centrifugation steps to the supernatant of the initial centrifugation, wherein a supernatant is used in each step. The removing cells or cell debris by centrifuging the biological fluid comprises low speed centrifugation. The removing cells or cell debris by centrifuging the biological fluid may comprise centrifugation which applies about 200 to 5,000 g for 1-10 minutes, or 100 to 1,200 g for 1-10 minutes.

The process above, wherein the one or more serial centrifugation steps each apply about 1,000 to 60,000 g for 1-100 minutes, or 1,000 to 3,000 g for 1-30 minutes to the supernatant from the previous step. The process above, comprising filtering the supernatant from a step of serial centrifugation with a filter for passing particles of less than a cutoff size; wherein the cutoff size is 1, or 2, or 3, or 4, or 5, or 6, or 10 micrometers, and
extracting extracellular matrix bodies trapped in the filter. The process above, comprising additional separating, isolating or enriching extracellular matrix bodies from a supernatant after centrifuging the biological fluid, wherein the additional separating, isolating or enriching is done by microfluidic device, centrifugation, chromatography, chemical precipitation, filtration, immuno- or affinity-capture, electrophoresis, AC electrokinetics, or a combination thereof.

The process above, wherein the separating, isolating or enriching captures at least a majority of the extracellular matrix bodies from the biological fluid. The process above, wherein the separating, isolating or enriching captures at least a majority of the extracellular matrix bodies from the biological fluid with an absence of cells. The process above, wherein the separating, isolating or enriching captures substantially all of the extracellular matrix bodies from the biological fluid. The process above, wherein the separating, isolating or enriching captures substantially all of the extracellular matrix bodies from the biological fluid with an absence of cells. The process above, wherein the level of separated, isolated or enriched extracellular matrix bodies is a biomarker for medical, diagnostic or prognostic information. The process above, wherein the separated, isolated or enriched extracellular matrix bodies comprise biomarkers in the form of a protein, a polypeptide, a lipid molecule, a lipoparticle, a carbohydrate, a nucleic acid molecule, or an expression level of a nucleic acid. The process above, wherein the separated, isolated or enriched extracellular matrix bodies comprise biomarkers in the form of extracellular proteins, RNA, DNA, or a protein in Table 1.

Embodiments of this invention further include The process above, comprising separating, isolating or enriching extracellular matrix bodies by:
re-suspending a pellet obtained from any preceding step;
filtering the re-suspension with a filter for passing particles of less than a cutoff size, wherein the cutoff size is 1, or 2, or 3, or 4, or 5, or 6, or 10 micrometers; and
extracting extracellular matrix bodies trapped in the filter. The process above, wherein the filtering captures at least a majority of the extracellular matrix bodies from the biological fluid, and the re-suspension is substantially free of particles smaller than about 1 micrometer. The process above, wherein the filtering captures substantially all of the extracellular matrix bodies from the biological fluid, and the re-suspension is substantially free of particles smaller than about 1 micrometer. The process above, comprising adding a reagent to a supernatant after the initial centrifugation, wherein the reagent is for precipitating the extracellular matrix bodies. The process above, wherein the extracellular matrix bodies are at least 2-fold, or at least 5-fold, or at least 10-fold enriched in concentration as compared to the biological fluid. The process above, wherein the extracellular matrix bodies are associated with a pathology or disease.

The process above, wherein the biological fluid is any one of whole blood, blood plasma, blood serum, cerebrospinal fluid, vitreous humor, aqueous humor, urine, saliva, sweat, tears, synovial fluid, pleural fluid, gastric fluid, peritoneal fluid, breast milk, nipple aspirate, ocular fluid, semen, amniotic fluid, lymph, bile, cerumen, chyle, chyme, endolymph, perilymph, exudates, feces, ejaculate, gastric acid, gastric juice, mucus, pericardial fluid, pus, rheum, sebum, serous fluid, smegma, sputum, synovial fluid, vaginal secretion, menstrual effluent, vomit and combinations thereof.

The biological fluid may preferably be any one of whole blood, blood plasma, blood serum, cerebrospinal fluid, vitreous humor, aqueous humor, urine, and saliva, and is more preferably blood plasma.

The process above, comprising determining a level of a biomarker of the separated, isolated or enriched extracellular matrix bodies.

The process above, wherein the biomarker is the level of the extracellular matrix bodies, or the level of a substance found in the extracellular matrix bodies, wherein the substance is a protein, a polypeptide, a lipid molecule, a lipoparticle, a carbohydrate, a nucleic acid molecule, or an expression level of a nucleic acid.

The process above, wherein the level of the extracellular matrix bodies is determined by microscopy.

The process above, wherein the level of the substance is determined by any of immunostaining, fluorescence assay, chelate complexation, quantitative HPLC, spectrophotometry, antibody array, Western blot, immunoassay, immunoprecipitation, ELISA, LC-MS, LC-MRM, radioimmunoassay, mass spectrometry, 2D gel mass spectrometry, LC-MS/MS, RT-PCR, nucleic acid assay, next generation sequencing, and combinations thereof.

This invention further contemplates processes for diagnosing, prognosing or monitoring a disease in a subject, the process comprising
separating, isolating or enriching extracellular matrix bodies in a biological fluid sample of the subject;
determining a level of one or more biomarkers based on the separated, isolated or enriched extracellular matrix bodies, wherein the biomarker is the level of the extracellular matrix bodies, or the level of a substance found in the extracellular matrix bodies, wherein the substance is a protein, a polypeptide, a lipid molecule, a lipoparticle, a carbohydrate, a nucleic acid molecule, or an expression level of a nucleic acid; and
comparing the levels of the biomarkers to reference levels based on a control group of subjects, and diagnosing, prognosing or monitoring the disease in the subject.

The process above, wherein the separated, isolated or enriched extracellular matrix bodies comprise biomarkers in the form of proteins, extracellular matrix proteins, polypeptides, lipids, lipoparticles, carbohydrates, nucleic acid molecules, DNA, or an expression level of a nucleic acid.

The process above, wherein the separating, isolating or enriching extracellular matrix bodies in a biological fluid sample of the subject comprises performing a process above.

The process above, comprising treating the subject for the disease by any one or more of surgery, drug therapy, therapeutic radiation, and chemotherapy.

Embodiments of this invention include compositions comprising extracellular matrix bodies isolated by the process above. The composition above, wherein the extracellular matrix bodies are associated with pathology of a disease. A composition above, for use in a method of therapy of a human or animal body.

This invention further includes methods for preparing a biological sample for a medical, diagnostic or prognostic use, the method may comprise isolating extracellular matrix bodies from the biological sample according to the process above, wherein the extracellular matrix bodies have a principal size from about 1 micrometer to 200 micrometers, or from about 4 micrometers to 200 micrometers. The method above, wherein the biological sample is composed of a bodily fluid. The method above, wherein the bodily fluid is any of whole blood, blood plasma, blood serum, cerebrospinal fluid, vitreous humor, aqueous humor, urine, saliva, sweat, tears, synovial fluid, pleural fluid, gastric fluid, peritoneal fluid, breast milk, nipple aspirate, ocular fluid, semen, amniotic fluid, lymph, bile, cerumen, chyle, chyme, endolymph, perilymph, exudates, feces, ejaculate, gastric acid, gastric juice, mucus, pericardial fluid, pus, rheum, sebum, serous fluid, smegma, sputum, synovial fluid, vaginal secretion, menstrual effluent, vomit and combinations thereof.

Embodiments of this invention include methods for preparing a sample by distinguishing extracellular matrix bodies in a biological fluid or material or tissue for a medical, diagnostic or prognostic use, the method comprising:
isolating extracellular matrix bodies from the biological sample according to the process above, wherein the extracellular matrix bodies have a principal size from about 1 micrometer to 200 micrometers, or from about 4 micrometers to 200 micrometers. The method above, wherein the biological sample is composed of a bodily fluid. The method above, wherein the bodily fluid is any of whole blood, blood plasma, blood serum, cerebrospinal fluid, vitreous humor, aqueous humor, urine, saliva, sweat, tears, synovial fluid, pleural fluid, gastric fluid, peritoneal fluid, breast milk, nipple aspirate, ocular fluid, semen, amniotic fluid, lymph, bile, cerumen, chyle, chyme, endolymph, perilymph, exudates, feces, ejaculate, gastric acid, gastric juice, mucus, pericardial fluid, pus, rheum, sebum, serous fluid, smegma, sputum, synovial fluid, vaginal secretion, menstrual effluent, vomit and combinations thereof.

This invention further contemplates methods for preparing a sample of extracellular matrix bodies in a biological fluid by fixation, the method comprising contacting the biological fluid with a non-reversible cross-linking agent which fixes the extracellular matrix bodies. The method above, wherein the non-reversible cross-linking agent is a water-soluble carbodiimide, a cyanogen halide, or a mixture thereof. The method above, wherein the non-reversible cross-linking agent is 1-ethyl-3-(3-dimethylaminopropyl)-carbodiimide, cyanogen bromide, cyanogen fluoride, cyanogen chloride, or cyanogen iodide. The method above, comprising contacting the biological fluid with an aldehyde-containing fixative agent. The method above, comprising detecting the fixed extracellular matrix bodies by microscopy, spectrophotometry, tomography, or magnetic resonance. The method above, wherein the biological fluid is any of whole blood, blood plasma, blood serum, cerebrospinal fluid, vitreous humor, aqueous humor, urine, saliva, sweat, tears, synovial fluid, pleural fluid, gastric fluid, peritoneal fluid, breast milk, nipple aspirate, ocular fluid, semen, amniotic fluid, lymph, bile, cerumen, chyle, chyme, endolymph, perilymph, exudates, feces, ejaculate, gastric acid, gastric juice, mucus, pericardial fluid, pus, rheum, sebum, serous fluid, smegma, sputum, synovial fluid, vaginal secretion, menstrual effluent, vomit and combinations thereof.

The non-reversible cross-linking agent may preferably be a water-soluble carbodiimide, for example 1-ethyl-3-(3-dimethylaminopropyl) carbodiimide (EDC).

This invention includes kits for fixing extracellular matrix bodies in a biological fluid, comprising:
a support substrate for holding the biological fluid; and
a non-reversible cross-linking agent. The kit above, wherein the non-reversible cross-linking agent is 1-ethyl-3-(3-dimethylaminopropyl)-carbodiimide, cyanogen bromide, cyanogen fluoride, cyanogen chloride, or cyanogen iodide, and comprising an aldehyde-containing fixative agent.

### BRIEF DESCRIPTION OF THE DRAWINGS

FIG. 1 shows a schematic of separation and isolation of extracellular matrix bodies from a bodily fluid by centrifugation.

FIG. 2 shows a schematic of methods for separating and isolating extracellular matrix bodies from a bodily fluid by centrifugation and filtration.

FIG. 3A shows a first step in serial centrifugation of a bodily fluid which can separate and isolate certain extracellular matrix bodies from a native bodily fluid.

FIG. 3B shows a step in serial centrifugation of a bodily fluid which can enrich certain extracellular matrix bodies from the supernatant of a previous step of centrifugation.

FIG. 3C shows a further step in serial centrifugation of a bodily fluid which can enrich certain extracellular matrix bodies from the supernatant of a previous step of centrifugation.

FIG. 3D shows a further step in serial centrifugation of a bodily fluid which can separate and isolate substantially all extracellular matrix bodies from a native bodily fluid into a pellet. The pellet can be resuspended to provide a fluid containing extracellular matrix bodies substantially free of small particles.

FIG. 4 shows a quantitative example of separating and enriching extracellular matrix bodies from a native human bodily fluid (plasma) by differential centrifugation. The relative quantities of extracellular matrix bodies isolated in a pellet (dashed line, open squares) and remaining in a supernatant (solid line, crosses) are shown.

FIG. 5 shows a representative micrograph of a diffuse extracellular matrix body detected in native human plasma by light microscopy of a sample fixed with EDC on a poly-l-lysine coated glass slide and stained with hematoxylin and eosin.

FIG. 6A shows a representative micrograph of extracellular matrix bodies isolated in native human plasma obtained from a subject having an internal disease. Image was obtained by light microscopy for extracellular matrix bodies fixed using EDC on a glass slide and stained with hematoxylin and eosin.

FIG. 6B shows a chart of the quantity by size of extracellular matrix bodies counted in native human plasma obtained from a non-disease subject by light microscopy of a sample fixed with EDC on a glass slide and stained with hematoxylin and eosin.

FIG. 6C shows a chart of the quantity of particles and their sizes for extracellular matrix bodies counted in native human plasma obtained from an internal disease subject by light microscopy fixed using EDC on a glass slide and stained with hematoxylin and eosin. The subject was newly diagnosed as having the internal disease.

FIG. 6D shows a chart of the quantity of particles and their sizes for extracellular matrix bodies counted in native human plasma obtained from a subject having the same internal disease as FIG. 6C, but having been diagnosed at a comparatively earlier date, and therefore having the internal disease for a longer period. Image was obtained by light microscopy fixed using EDC on a glass slide and stained with hematoxylin and eosin.

FIG. 6E shows a combined chart of FIG. 6B, FIG. 6C and FIG. 6D.

FIG. 7 shows a representative micrograph of native extracellular matrix bodies detected in bovine vitreous humor by light microscopy.

FIG. 8A shows a graph of isolation of extracellular matrix bodies in bovine vitreous humor by differential centrifugation. This graph shows the level of extracellular matrix bodies in the supernatant (S, filled triangles, dashed regression line) and pellet (P, open circles, solid regression line) after centrifugation at 1,000 to 12,000 g. Extracellular matrix bodies were separated to a high degree by differential centrifugation at about 6,000 to 12,000 g.

FIG. 8B shows a representative micrograph of extracellular matrix bodies isolated in bovine vitreous humor by centrifugation. This image shows extracellular matrix bodies in the supernatant after centrifugation at 500 g. Image was obtained by light microscopy of a sample fixed with EDC on a glass slide and stained with hematoxylin and eosin. Scale bars are 20 um.

FIG. 8C shows a representative micrograph of extracellular matrix bodies isolated in bovine vitreous humor by centrifugation. This image shows extracellular matrix bodies re-suspended from a pellet after centrifugation at 500 g. Image was obtained by light microscopy of a sample fixed with EDC on a glass slide and stained with hematoxylin and eosin. Scale bars are 20 um.

FIG. 8D shows an expansion of the micrograph in FIG. 8B.

FIG. 8E shows an expansion of the micrograph in FIG. 8C.

FIG. 8F shows a representative micrograph of extracellular matrix bodies isolated in bovine vitreous humor by centrifugation. This image shows extracellular matrix bodies in the supernatant after centrifugation at 500 g. Image was obtained by light microscopy of a sample fixed with EDC on a glass slide and stained with alcian blue. Scale bars are 20 um.

FIG. 8G shows a representative micrograph of extracellular matrix bodies isolated in bovine vitreous humor by centrifugation. This image shows extracellular matrix bodies re-suspended from a pellet after centrifugation at 500 g. Image was obtained by light microscopy of a sample fixed with EDC on a glass slide and stained with alcian blue. Scale bars are 20 um.

FIG. 8H shows an expansion of the micrograph in FIG. 8F.

FIG. 8I shows an expansion of the micrograph in FIG. 8G.

FIG. 9A shows a representative micrograph of extracellular matrix bodies isolated in bovine vitreous humor by centrifugation. This image shows extracellular matrix bodies in the supernatant after centrifugation at 1,000 g. Image was obtained by light microscopy and samples fixed with EDC on a glass slide and stained with hematoxylin and eosin. Scale bars are 20 um.

FIG. 9B shows a representative micrograph of extracellular matrix bodies isolated in bovine vitreous humor by centrifugation. This image shows extracellular matrix bodies re-suspended from a pellet after centrifugation at 1,000 g. Image was obtained by light microscopy and samples fixed with EDC, placed on a glass slide and stained with hematoxylin and eosin. Scale bars are 20 μm.

FIG. 9C shows an expansion of the micrograph in FIG. 9A.

FIG. 9D shows an expansion of the micrograph in FIG. 9B.

FIG. 9E shows a representative micrograph of extracellular matrix bodies isolated in bovine vitreous humor by centrifugation. This image shows extracellular matrix bodies in the supernatant after centrifugation at 1,000 g. Image was obtained by light microscopy of the sample fixed with EDC on a glass slide and stained with alcian blue. Scale bars are 20 um.

FIG. 9F shows a representative micrograph of extracellular matrix bodies isolated in bovine vitreous humor by centrifugation. This image shows extracellular matrix bodies re-suspended from a pellet after centrifugation at 1,000 g. Image was obtained by light microscopy of the sample fixed with EDC on a glass slide and stained with alcian blue. Scale bars are 20 um.

FIG. 9G shows an expansion of the micrograph in FIG. 9E.

FIG. 9H shows an expansion of the micrograph in FIG. 9F.

FIG. 10A shows a representative micrograph of extracellular matrix bodies isolated in bovine vitreous humor by centrifugation. This image shows extracellular matrix bodies in the supernatant after centrifugation at 2,000 g. Image was obtained by light microscopy with the sample fixed with EDC on a glass slide and stained with hematoxylin and eosin. Scale bars are 20 um.

FIG. 10B shows a representative micrograph of extracellular matrix bodies isolated in bovine vitreous humor by centrifugation. This image shows extracellular matrix bodies re-suspended from a pellet after centrifugation at 2,000 g. Image was obtained by light microscopy with the sample fixed with EDC on a glass slide and stained with hematoxylin and eosin. Scale bars are 20 μm.

FIG. 10C shows an expansion of the micrograph in FIG. 10A.

FIG. 10D shows an expansion of the micrograph in FIG. 10B.

FIG. 10E shows a representative micrograph of extracellular matrix bodies isolated in bovine vitreous humor by centrifugation. This image shows extracellular matrix bodies in the supernatant after centrifugation at 2,000 g. Image was obtained by light microscopy with the sample fixed with EDC on a glass slide and stained with alcian blue. Scale bars are 20 um.

FIG. 10F shows a representative micrograph of extracellular matrix bodies isolated in bovine vitreous humor by centrifugation. This image shows extracellular matrix bodies re-suspended from a pellet after centrifugation at 2,000 g. Image was obtained by light microscopy with the sample fixed with EDC on a glass slide and stained with alcian blue. Scale bars are 20 um.

FIG. 10G shows an expansion of the micrograph in FIG. 10E.

FIG. 10H shows an expansion of the micrograph in FIG. 10F.

FIG. 11A shows a representative micrograph of extracellular matrix bodies isolated in bovine vitreous humor by centrifugation. This image shows extracellular matrix bodies in the supernatant after centrifugation at 3,000 g. Image was obtained by light microscopy with a sample fixed with EDC on a glass slide and stained with hematoxylin and eosin. Scale bars are 20 um.

FIG. 11B shows a representative micrograph of extracellular matrix bodies isolated in bovine vitreous humor by centrifugation. This image shows extracellular matrix bodies re-suspended from a pellet after centrifugation at 3,000 g. Image was obtained by light microscopy of a sample fixed with EDC on a glass slide and stained with hematoxylin and eosin. Scale bars are 20 μm.

FIG. 11C shows an expansion of the micrograph in FIG. 11A.

FIG. 11D shows an expansion of the micrograph in FIG. 11B.

FIG. 11E shows a representative micrograph of extracellular matrix bodies isolated in bovine vitreous humor by centrifugation. This image shows extracellular matrix bodies in the supernatant after centrifugation at 3,000 g. Image was obtained by light microscopy of a sample fixed with EDC on glass slide and stained with alcian blue. Scale bars are 20 um.

FIG. 11F shows a representative micrograph of extracellular matrix bodies isolated in bovine vitreous humor by centrifugation. This image shows extracellular matrix bodies re-suspended from a pellet after centrifugation at 3,000 g. Image was obtained by light microscopy of a sample fixed with EDC on a glass slide and stained with alcian blue. Scale bars are 20 um.

FIG. 12A shows a representative micrograph of extracellular matrix bodies isolated in bovine vitreous humor by centrifugation. This image shows extracellular matrix bodies in the supernatant after centrifugation at 4,000 g. Image was obtained by light microscopy of a sample fixed with EDC on a glass slide and stained with hematoxylin and eosin. Scale bars are 20 um.

FIG. 12B shows a representative micrograph of extracellular matrix bodies isolated in bovine vitreous humor by centrifugation. This image shows extracellular matrix bodies re-suspended from a pellet after centrifugation at 4,000 g. Image was obtained by light microscopy of a sample fixed with EDC on a glass slide and stained with hematoxylin and eosin. Scale bars are 20 μm.

FIG. 12C shows an expansion of the micrograph in FIG. 12A.

FIG. 12D shows an expansion of the micrograph in FIG. 12B.

FIG. 12E shows a representative micrograph of extracellular matrix bodies isolated in bovine vitreous humor by centrifugation. This image shows extracellular matrix bodies in the supernatant after centrifugation at 4,000 g. Image was obtained by light microscopy of a sample fixed with EDC on a glass slide and stained with alcian blue. Scale bars are 20 um.

FIG. 12F shows a representative micrograph of extracellular matrix bodies isolated in bovine vitreous humor by centrifugation. This image shows extracellular matrix bodies re-suspended from a pellet after centrifugation at 4,000 g. Image was obtained by light microscopy of a sample fixed with EDC on a glass slide and stained with alcian blue. Scale bars are 20 um.

FIG. 12G shows an expansion of the micrograph in FIG. 12E.

FIG. 12H shows an expansion of the micrograph in FIG. 12F.

FIG. 13A shows a representative micrograph of extracellular matrix bodies isolated in bovine vitreous humor by centrifugation. This image shows extracellular matrix bodies in the supernatant after centrifugation at 5,000 g. Image was obtained by light microscopy of a sample fixed with EDC on a glass slide and stained with hematoxylin and eosin. Scale bars are 20 um.

FIG. 13B shows a representative micrograph of extracellular matrix bodies isolated in bovine vitreous humor by centrifugation. This image shows extracellular matrix bodies re-suspended from a pellet after centrifugation at 5,000 g. Image was obtained by light microscopy of a sample fixed with EDC on a glass slide and stained with hematoxylin and eosin. Scale bars are 20 μm.

FIG. 13C shows an expansion of the micrograph in FIG. 13A.

FIG. 13D shows an expansion of the micrograph in FIG. 13B.

FIG. 13E shows a representative micrograph of extracellular matrix bodies isolated in bovine vitreous humor by centrifugation. This image shows extracellular matrix bodies in the supernatant after centrifugation at 5,000 g. Image was obtained by light microscopy of a sample fixed with EDC on a glass slide and stained with alcian blue. Scale bars are 20 um.

FIG. 13F shows a representative micrograph of extracellular matrix bodies isolated in bovine vitreous humor by centrifugation. This image shows extracellular matrix bodies re-suspended from a pellet after centrifugation at 5,000 g. Image was obtained by light microscopy of a sample fixed with EDC on a glass slide and stained with alcian blue. Scale bars are 20 um.

FIG. 13G shows an expansion of the micrograph in FIG. 13E.

FIG. 13H shows an expansion of the micrograph in FIG. 13F.

FIG. 14A shows a representative micrograph of extracellular matrix bodies isolated in bovine vitreous humor by centrifugation. This image shows extracellular matrix bodies in the supernatant after centrifugation at 10,000 g. Image was obtained by light microscopy of a sample fixed with EDC on a glass slide and stained with hematoxylin and eosin. Scale bars are 20 um.

FIG. 14B shows a representative micrograph of extracellular matrix bodies isolated in bovine vitreous humor by centrifugation. This image shows extracellular matrix bodies re-suspended from a pellet after centrifugation at 10,000 g. Image was obtained by light microscopy of a sample fixed with EDC on a glass slide and stained with hematoxylin and eosin. Scale bars are 20 μm.

FIG. 14C shows a representative micrograph of extracellular matrix bodies isolated in bovine vitreous humor by centrifugation. This image shows extracellular matrix bodies in the supernatant after centrifugation at 10,000 g. Image was obtained by light microscopy of a sample fixed with EDC on a glass slide and stained with alcian blue. Scale bars are 20 um.

FIG. 14D shows a representative micrograph of extracellular matrix bodies isolated in bovine vitreous humor by centrifugation. This image shows extracellular matrix bodies re-suspended from a pellet after centrifugation at 10,000 g. Image was obtained by light microscopy of a sample fixed with EDC on a poly-l-lysine coated glass slide and stained with alcian blue. Scale bars are 20 μm.

FIG. 15A shows a representative micrograph of extracellular matrix bodies isolated in bovine vitreous humor by centrifugation. This image shows extracellular matrix bodies in the supernatant after centrifugation at 12,000 g. Image was obtained by light microscopy of a sample fixed with EDC on a glass slide and stained with hematoxylin and eosin. Scale bars are 20 um.

FIG. 15B shows a representative micrograph of extracellular matrix bodies isolated in bovine vitreous humor by centrifugation. This image shows extracellular matrix bodies re-suspended from a pellet after centrifugation at 12,000 g. Image was obtained by light microscopy of a sample fixed with EDC on a glass slide and stained with hematoxylin and eosin. Scale bars are 20 μm.

FIG. 15C shows an expansion of the micrograph in FIG. 15A.

FIG. 15D shows an expansion of the micrograph in FIG. 15B.

FIG. 15E shows a representative micrograph of extracellular matrix bodies isolated in bovine vitreous humor by centrifugation. This image shows extracellular matrix bodies in the supernatant after centrifugation at 12,000 g. Image was obtained by light microscopy of a sample fixed with EDC on a glass slide and stained with alcian blue. Scale bars are 20 um.

FIG. 15F shows a representative micrograph of extracellular matrix bodies isolated in bovine vitreous humor by centrifugation. This image shows extracellular matrix bodies re-suspended from a pellet after centrifugation at 12,000 g. Image was obtained by light microscopy of a sample fixed with EDC on a glass slide and stained with alcian blue. Scale bars are 20 um.

FIG. 15G shows an expansion of the micrograph in FIG. 15E.

FIG. 15H shows an expansion of the micrograph in FIG. 15F.

FIG. 16A shows an aliquot of human aqueous humor biofluid fixed on a poly-l-lysine coated formvar TEM grid with glutaraldehyde fixation agent and negative staining with a uranyl acetate solution. The transmission electron microscopy image showed that substantially no biological material was fixed and detected on the grid.

FIG. 16B shows an aliquot of human aqueous humor biofluid fixed on a poly-l-lysine coated formvar TEM grid with EDC fixation agent, and subsequently glutaraldehyde fixation, with uranyl acetate negative staining. The transmission electron microscopy image showed that biological material was fixed and detected on the grid. The biological material was an abundant number of extracellular matrix bodies.

FIG. 16C shows a comparison of the quantity of extracellular matrix bodies counted in images of three native human aqueous humor samples (A, B, C) obtained by the carbodiimide-fixation method of this invention (white bar) as compared to conventional fixation (solid bar). The detected quantities of extracellular matrix bodies are surprisingly enhanced by this invention.

FIG. 17A shows a representative micrograph of extracellular matrix bodies isolated in bovine vitreous humor by serial centrifugation. This image shows extracellular matrix bodies in the supernatant after centrifugation at 500 g. Image was obtained by light microscopy of a sample fixed with EDC on a glass slide and stained with hematoxylin and eosin. Scale bars are 20 um.

FIG. 17B shows a representative micrograph of extracellular matrix bodies isolated in bovine vitreous humor by serial centrifugation. This image shows extracellular matrix bodies re-suspended from a pellet after centrifugation at 500 g. Image was obtained by light microscopy of a sample fixed with EDC on a glass slide and stained with hematoxylin and eosin. Scale bars are 20 μm.

FIG. 18A shows a representative micrograph of extracellular matrix bodies isolated in bovine vitreous humor by serial centrifugation. This image shows extracellular matrix bodies in the supernatant after centrifugation at 1,000 g. Image was obtained by light microscopy of a sample fixed with EDC on a glass slide and stained with hematoxylin and eosin. Scale bars are 20 um.

FIG. 18B shows a representative micrograph of extracellular matrix bodies isolated in bovine vitreous humor by serial centrifugation. This image shows extracellular matrix bodies re-suspended from a pellet after centrifugation at 1,000 g. Image was obtained by light microscopy of a sample fixed with EDC on a glass slide and stained with hematoxylin and eosin. Scale bars are 20 μm.

FIG. 19A shows a representative micrograph of extracellular matrix bodies isolated in bovine vitreous humor by serial centrifugation. This image shows extracellular matrix bodies in the supernatant after centrifugation at 2,000 g. Image was obtained by light microscopy of a sample fixed with EDC on a glass slide and stained with hematoxylin and eosin. Scale bars are 20 um.

FIG. 19B shows a representative micrograph of extracellular matrix bodies isolated in bovine vitreous humor by serial centrifugation. This image shows extracellular matrix bodies re-suspended from a pellet after centrifugation at 2,000 g. Image was obtained by light microscopy of a sample fixed with EDC on a glass slide and stained with hematoxylin and eosin. Scale bars are 20 μm.

FIG. 20A shows a representative micrograph of extracellular matrix bodies isolated in bovine vitreous humor by serial centrifugation. This image shows extracellular matrix bodies in the supernatant after centrifugation at 3,000 g. Image was obtained by light microscopy of a sample fixed with EDC on a glass slide and stained with hematoxylin and eosin. Scale bars are 20 um.

FIG. 20B shows a representative micrograph of extracellular matrix bodies isolated in bovine vitreous humor by serial centrifugation. This image shows extracellular matrix bodies re-suspended from a pellet after centrifugation at 3,000 g. Image was obtained by light microscopy of a sample fixed with EDC on a glass slide and stained with hematoxylin and eosin. Scale bars are 20 μm.

FIG. 21A shows a representative micrograph of extracellular matrix bodies isolated in bovine vitreous humor by serial centrifugation. This image shows extracellular matrix bodies in the supernatant after centrifugation at 4,000 g. Image was obtained by light microscopy of a sample fixed with EDC on a glass slide and stained with hematoxylin and eosin. Scale bars are 20 um.

FIG. 21B shows a representative micrograph of extracellular matrix bodies isolated in bovine vitreous humor by serial centrifugation. This image shows extracellular matrix bodies re-suspended from a pellet after centrifugation at 4,000 g. Image was obtained by light microscopy of a sample fixed with EDC on a glass slide and stained with hematoxylin and eosin. Scale bars are 20 μm.

FIG. 22A shows a representative micrograph of extracellular matrix bodies isolated in bovine vitreous humor by serial centrifugation. This image shows extracellular matrix bodies in the supernatant after centrifugation at 5,000 g. Image was obtained by light microscopy of a sample fixed with EDC on a glass slide and stained with hematoxylin and eosin. Scale bars are 20 um.

FIG. 22B shows a representative micrograph of extracellular matrix bodies isolated in bovine vitreous humor by serial centrifugation. This image shows extracellular matrix bodies re-suspended from a pellet after centrifugation at 5,000 g. Image was obtained by light microscopy of a sample fixed with EDC on a glass slide and stained with hematoxylin and eosin. Scale bars are 20 μm.

FIG. 23A shows a representative micrograph of extracellular matrix bodies isolated in bovine vitreous humor by serial centrifugation. This image shows extracellular matrix bodies in the supernatant after centrifugation at 10,000 g. Image was obtained by light microscopy of a sample fixed with EDC on a glass slide and stained with hematoxylin and eosin. Scale bars are 20 um.

FIG. 23B shows a representative micrograph of extracellular matrix bodies isolated in bovine vitreous humor by serial centrifugation. This image shows extracellular matrix bodies re-suspended from a pellet after centrifugation at 10,000 g. Image was obtained by light microscopy of a sample fixed with EDC on a glass slide and stained with hematoxylin and eosin. Scale bars are 20 μm.

FIG. 24A shows a representative micrograph of extracellular matrix bodies isolated in bovine vitreous humor by serial centrifugation. This image shows extracellular matrix bodies in the supernatant after centrifugation at 12,000 g. Image was obtained by light microscopy of a sample fixed with EDC on a glass slide and stained with hematoxylin and eosin. Scale bars are 20 um.

FIG. 24B shows a representative micrograph of extracellular matrix bodies isolated in bovine vitreous humor by serial centrifugation. This image shows extracellular matrix bodies re-suspended from a pellet after centrifugation at 12,000 g. Image was obtained by light microscopy of a sample fixed with EDC on a glass slide and stained with hematoxylin and eosin. Scale bars are 20 μm.

FIG. 25 shows a particle analysis of extracellular matrix bodies isolated in bovine vitreous humor by centrifugation. Extracellular matrix bodies were analyzed in the supernatant (filled triangles) and the pellet (open circles) after centrifugation at 1,000 g.

FIG. 26 shows a particle analysis of extracellular matrix bodies isolated in bovine vitreous humor by centrifugation. Extracellular matrix bodies were analyzed in the supernatant (filled triangles) and the pellet (open circles) after centrifugation at 2,000 g.

FIG. 27 shows a particle analysis of extracellular matrix bodies isolated in bovine vitreous humor by centrifugation. Extracellular matrix bodies were analyzed in the supernatant (filled triangles) and the pellet (open circles) after centrifugation at 4,000 g.

FIG. 28 shows a particle analysis of extracellular matrix bodies isolated in bovine vitreous humor by centrifugation. Extracellular matrix bodies were analyzed in the supernatant (filled triangles) and the pellet (open circles) after centrifugation at 5,000 g.

FIG. 29 shows a particle analysis of extracellular matrix bodies isolated in bovine vitreous humor by centrifugation. Extracellular matrix bodies were analyzed in the supernatant (filled triangles) and the pellet (open circles) after centrifugation at 10,000 g.

FIG. 30 shows a particle analysis of extracellular matrix bodies isolated in bovine vitreous humor by centrifugation. Extracellular matrix bodies were analyzed in the supernatant (filled triangles) and the pellet (open circles) after centrifugation at 12,000 g.

FIG. 31 shows a representative micrograph of bovine vitreous humor processed with centrifugation. The bovine vitreous humor was centrifuged at 12,000 g, followed by resuspending the pellet in 400 μl of phosphate buffer saline. This image showed that significant amounts of extracellular matrix bodies were obtained from the resuspended pellet. Image was obtained by light microscopy of a sample fixed with EDC on a glass slide and stained with hematoxylin and eosin.

FIG. 32 shows an expansion of the micrograph in FIG. 31.

FIG. 33 shows a representative micrograph of bovine vitreous humor processed with centrifugation and membrane filtration. The bovine vitreous humor was centrifuged at 12,000 g, followed by resuspending the pellet in 400 μl of phosphate buffer saline and filtering with a cellulose acetate membrane with a 0.45 μm pore size. This image showed that substantially all extracellular matrix bodies were removed from the filtrate fraction distal to the 0.45 μm membrane. Image was obtained by light microscopy of a sample fixed with EDC on a glass slide and stained with hematoxylin and eosin.

FIG. 34 shows an expansion of the micrograph in FIG. 33.

FIG. 35 shows a representative micrograph of bovine vitreous humor extracted from the filter after processing with centrifugation and membrane filtration. The bovine vitreous humor was centrifuged at 12,000 g, followed by resuspending the pellet in 400 μl of phosphate buffer saline and filtering the suspension with a cellulose acetate membrane with a 0.45 μm pore size. Extracellular matrix bodies we recovered by extracting the material held on the proximal surface of the 0.45-micron filter. This image shows the presence of extracellular matrix bodies extracted from proximal surface of the 0.45-micron filter.

FIG. 36 shows an expansion of the micrograph in FIG. 35.

FIG. 37A shows an embodiment of a method for quantifying extracellular matrix bodies in a bodily fluid, bovine vitreous humor. The lengths of extracellular matrix bodies were measured by segmenting the image to identify and measure uninterrupted lengths of the extracellular matrix bodies (white lines).

FIG. 37B shows an embodiment of a method for quantifying extracellular matrix bodies in a bodily fluid, bovine vitreous humor. The lengths of extracellular matrix bodies can be measured by segmenting the image to identify and measure uninterrupted lengths of the extracellular matrix bodies (white lines).

FIG. 37C shows an embodiment of a method for quantifying extracellular matrix bodies in a bodily fluid, bovine vitreous humor. The lengths of extracellular matrix bodies can be measured by segmenting the image to identify and measure uninterrupted lengths of the extracellular matrix bodies (white lines). Scale bars are 20 um.

FIG. 38 shows quantification of extracellular matrix bodies in a method of this invention. Bovine vitreous humor was centrifuged at 12,000 g, followed by resuspending the pellet in 400 μl of phosphate buffer saline and filtering the suspension with a cellulose acetate membrane with a 0.45 μm pore size. Extracellular matrix bodies we recovered by extracting the material held on the proximal surface of the 0.45-micron filter. The extracted isolate contained 65% of the extracellular matrix bodies. The filtrate contained 35% of the extracellular matrix bodies.

FIG. 39 shows quantification of extracellular matrix bodies in a method of this invention. Bovine vitreous humor was centrifuged at 12,000 g, followed by resuspending the pellet in 400 μl of phosphate buffer saline and filtering the suspension with a cellulose acetate membrane with a 0.45 μm pore size. Extracellular matrix bodies we recovered by extracting the material held on the proximal surface of the 0.45-micron filter. The extracted isolate extracellular matrix bodies (open circles) were larger in average body length than extracellular matrix bodies obtained from the filtrate (filled triangles).

FIG. 40 shows quantification of extracellular matrix bodies in a method of this invention. Bovine vitreous humor was centrifuged at 12,000 g, followed by resuspending the pellet in 400 μl of phosphate buffer saline and filtering the suspension with a cellulose acetate membrane with a 0.45 μm pore size. Extracellular matrix bodies we recovered by extracting the material held on the proximal surface of the 0.45-micron filter. The extracted isolate extracellular matrix bodies (open circles) comprised significantly greater total body length than extracellular matrix bodies obtained from the filtrate (filled triangles).

FIG. 41A shows a representative photomicrographic of the enrichment of extracellular matrix bodies by filtration from a biological fluid, bovine vitreous humor, using a cellulose acetate membrane filter with a pore size of 0.22 micron filter. Extracellular matrix bodies were recovered by reversing the filter and extracting material from the proximal side of the filter to obtain an isolate. Image was taken on a glass slide with EDC fixation. Scale bars are 20 um.

FIG. 41B shows a representative photomicrograph of the filtrate from a biological fluid, bovine vitreous humor, using a cellulose acetate membrane filter with a pore size of 0.22 micron filter. Relatively few extracellular matrix bodies, if any, were found in the filtrate. Scale bars are 20 μm.

FIG. 42A shows an expansion of the micrograph in FIG. 41A.

FIG. 42B shows an expansion of the micrograph in FIG. 41B.

FIG. 43 shows a particle analysis of extracellular matrix bodies enriched from bovine vitreous humor by filtration using a cellulose acetate membrane filter with a pore size of 0.22 micron. Extracellular matrix bodies were recovered by reversing the filter and extracting material from the proximal side of the filter to obtain an isolate by reverse elution. A greater abundance of extracellular matrix bodies was found in the isolate (filled circles) as compared to the filtrate (open circles).

FIG. 44 shows a graphical representation of the enrichment of extracellular matrix bodies by filtration from a biological fluid, bovine vitreous humor, using a cellulose acetate membrane filter with a pore size of 0.45 micron filter. A greater abundance of extracellular matrix bodies was found in the isolate as compared to the filtrate.

FIG. 45A shows a representative photomicrographic of the enrichment of extracellular matrix bodies by filtration from a biological fluid, bovine vitreous humor, using a cellulose acetate membrane filter with a pore size of 0.22 micron filter. Extracellular matrix bodies were recovered by reversing the filter and extracting material from the proximal side of the filter to obtain an isolate. Image was taken on a glass slide with EDC fixation. Scale bars are 20 um.

FIG. 45B shows a representative photomicrograph of the filtrate from a biological fluid, bovine vitreous humor, using a cellulose acetate membrane filter with a pore size of 0.45 micron filter. Relatively few extracellular matrix bodies, if any, were found in the filtrate. Scale bars are 20 μm.

FIG. 46A shows an expansion of the micrograph in FIG. 45A.

FIG. 46B shows an expansion of the micrograph in FIG. 45B.

FIG. 47 shows a graphical representation of the enrichment of extracellular matrix bodies by filtration from a biological fluid, bovine vitreous humor, using a cellulose acetate membrane filter with a pore size of 0.45 micron filter. A greater abundance of extracellular matrix bodies were found in the isolate (filled circles) as compared to the filtrate (open circles). Extracellular matrix bodies were quantified by analyzing multiple photographic images (n=3). Analysis was performed in ImageJ for each 20× image. The length of extracellular matrix bodies were measured using a freehand line tool and fifty measurements were taken for each image.

FIG. 48 shows a graphical representation of the enrichment of extracellular matrix bodies by filtration from a biological fluid, bovine vitreous humor, using a cellulose acetate membrane filter with a pore size of 0.45 micron filter. A greater abundance of extracellular matrix bodies were found in the isolate as compared to the filtrate.

FIG. 49A shows a representative photomicrographic of the enrichment of extracellular matrix bodies by filtration from a biological fluid, bovine vitreous humor, using a cellulose acetate membrane filter with a pore size of 1 micron filter. Extracellular matrix bodies were recovered by reversing the filter and extracting material from the proximal side of the filter to obtain an isolate. Image was taken on a glass slide with EDC fixation. Scale bars are 20 um.

FIG. 49B shows a representative photomicrograph of the filtrate from a biological fluid, bovine vitreous humor, using a cellulose acetate membrane filter with a pore size of 1 micron filter. Relatively few extracellular matrix bodies, if any, were found in the filtrate. Scale bars are 20 um.

FIG. 50A shows an expansion of the micrograph in FIG. 49A.

FIG. 50B shows an expansion of the micrograph in FIG. 49B.

FIG. 51 shows a graphical representation of the enrichment of extracellular matrix bodies by filtration from a biological fluid, bovine vitreous humor, using a cellulose acetate membrane filter with a pore size of 1 micron filter. A greater abundance of extracellular matrix bodies were found in the isolate (filled circles) as compared to the filtrate (open circles).

FIG. 52 shows a graphical representation of the enrichment of extracellular matrix bodies by filtration from a biological fluid, bovine vitreous humor, using a cellulose acetate membrane filter with a pore size of 1 micron filter. A greater abundance of extracellular matrix bodies were found in the isolate as compared to the filtrate.

FIG. 53A shows a representative photomicrographic of the enrichment of extracellular matrix bodies by filtration from a biological fluid, bovine vitreous humor, using a cellulose acetate membrane filter with a pore size of 5 micron filter. Extracellular matrix bodies were recovered by reversing the filter and extracting material from the proximal side of the filter to obtain an isolate. Image was taken on a glass slide with EDC fixation. Scale bars are 20 um.

FIG. 53B shows a representative photomicrograph of the filtrate from a biological fluid, bovine vitreous humor, using a cellulose acetate membrane filter with a pore size of 5 micron filter. Relatively fewer extracellular matrix bodies were found in the filtrate as compared to the isolate. Scale bars are 20 um.

FIG. 54A shows an expansion of the micrograph in FIG. 49A.

FIG. 54B shows an expansion of the micrograph in FIG. 49B.

FIG. 55 shows a graphical representation of the enrichment of extracellular matrix bodies by filtration from a biological fluid, bovine vitreous humor, using a cellulose acetate membrane filter with a pore size of 1 micron filter. A greater abundance of extracellular matrix bodies were found in the isolate (filled circles) as compared to the filtrate (open circles).

FIG. 56 shows a graphical representation of the enrichment of extracellular matrix bodies by filtration from a biological fluid, bovine vitreous humor, using a cellulose acetate membrane filter with a pore size of 1 micron filter. A greater abundance of extracellular matrix bodies were found in the isolate as compared to the filtrate.

FIG. 57A shows a representative photomicrographic of the enrichment of extracellular matrix bodies by filtration from a biological fluid, bovine vitreous humor, using a cellulose acetate membrane filter with a pore size of 10 micron filter. Extracellular matrix bodies were recovered by reversing the filter and extracting material from the proximal side of the filter to obtain an isolate. Image was taken on a glass slide with EDC fixation. Scale bars are 20 um.

FIG. 57B shows a representative photomicrograph of the filtrate from a biological fluid, bovine vitreous humor, using a cellulose acetate membrane filter with a pore size of 10 micron filter. Relatively fewer extracellular matrix bodies were found in the filtrate as compared to the isolate. Scale bars are 20 um.

FIG. 58A shows an expansion of the micrograph in FIG. 57A.

FIG. 58B shows an expansion of the micrograph in FIG. 57B.

FIG. 59 shows a graphical representation of the enrichment of extracellular matrix bodies by filtration from a biological fluid, bovine vitreous humor, using a cellulose acetate membrane filter with a pore size of 10 micron filter. A greater abundance of extracellular matrix bodies were found in the isolate (filled circles) as compared to the filtrate (open circles).

FIG. 60 shows a graphical representation of the enrichment of extracellular matrix bodies by filtration from a biological fluid, bovine vitreous humor, using a cellulose acetate membrane filter with a pore size of 10 micron filter, shown I FIG. 59. A greater abundance of extracellular matrix bodies were found in the isolate as compared to the filtrate.

FIG. 61A shows a representative photomicrographic of the enrichment of extracellular matrix bodies by filtration from a biological fluid, bovine vitreous humor, using a cellulose acetate membrane filter with a pore size of 0.22 micron filter. Extracellular matrix bodies were recovered by reversing the filter and extracting material from the proximal side of the filter to obtain an isolate. Image was taken on a glass slide with EDC fixation. Scale bars are 20 um. This image shows the filtrate.

FIG. 61B shows a representative photomicrograph of the isolate from a biological fluid, bovine vitreous humor, using a cellulose acetate membrane filter with a pore size of 0.22 micron filter. Scale bars are 20 um. Relatively few extracellular matrix bodies, if any, were found in the isolate as compared to the filtrate.

FIG. 62A shows an expansion of the micrograph in FIG. 61A.

FIG. 62B shows an expansion of the micrograph in FIG. 61B.

FIG. 63 shows a graphical representation of the enrichment of extracellular matrix bodies by filtration from a biological fluid, bovine vitreous humor, using a cellulose acetate membrane filter with a pore size of 0.22 micron filter. A greater abundance of extracellular matrix bodies were found in the isolate (filled circles) as compared to the filtrate (open circles).

FIG. 64A shows a representative photomicrographic of the enrichment of extracellular matrix bodies by serial (repeated) filtration from a biological fluid, bovine vitreous humor, using a cellulose acetate membrane filter, first with a pore size of 0.22 micron, and second with a pore size of 0.45 micron. Extracellular matrix bodies were recovered by reversing the filter and extracting material from the proximal side of the filter to obtain an isolate. Image was taken on a glass slide with EDC fixation. Scale bars are 20 um. This image shows the double isolate being the isolate after filtration at 0.22, then 0.45 micron.

FIG. 64B shows a representative photomicrographic of the enrichment of extracellular matrix bodies by serial (repeated) filtration from a biological fluid, bovine vitreous humor, using a cellulose acetate membrane filter, first with a pore size of 0.22 micron, and second with a pore size of 0.45 micron. Extracellular matrix bodies were recovered by reversing the filter and extracting material from the proximal side of the filter to obtain an isolate. Image was taken on a glass slide with EDC fixation. Scale bars are 20 um. This image shows the filtrate after filtration at 0.22, then 0.45 micron. Relatively few extracellular matrix bodies, if any, were found in the double filtrate as compared to the double isolate.

FIG. 65A shows an expansion of the micrograph in FIG. 64A.

FIG. 65B shows an expansion of the micrograph in FIG. 64B.

FIG. 66 shows a graphical representation of the enrichment of extracellular matrix bodies by serial filtration from a biological fluid, bovine vitreous humor, using a cellulose acetate membrane filter, first with a pore size of 0.22 micron, and second with a pore size of 0.45 micron. A greater abundance of extracellular matrix bodies were found in the double isolate (filled circles) as compared to the double filtrate (open circles).

FIG. 67 shows a graphical representation of the enrichment of extracellular matrix bodies by serial filtration from a biological fluid, bovine vitreous humor, using a cellulose acetate membrane filter, first with a pore size of 0.22 micron, and second with a pore size of 0.45 micron. A greater abundance of extracellular matrix bodies were found in the isolate as compared to the filtrate in each step of the serial filtration.

FIG. 68 shows a graphical representation of the enrichment of extracellular matrix bodies by serial filtration from a biological fluid, bovine vitreous humor, using a cellulose acetate membrane filter, first with a pore size of 0.22 micron, and second with a pore size of 0.45 micron. A multi-fold abundance of extracellular matrix bodies were found in the final isolate as compared to the final filtrate after serial filtration.

FIG. 69 shows a representative photomicrographic of extracellular matrix bodies isolated by membrane filtration from a biological fluid, human cerebrospinal fluid (CSF). CSF was filtered through a 0.45 μm syringe-tip filter. Extracellular matrix bodies were recovered from the proximal side of the membrane and fixed to a glass slide by an EDC-glutaraldehyde fixation protocol. The extracellular matrix bodies were stained with a monoclonal fluorescent-conjugated antibody (594) for fibronectin, an extracellular matrix protein. The slide was imaged by microscopy. Black lines and features in the image of FIG. 69 correspond to stained substances. This image shows that extracellular matrix bodies can be isolated from human cerebrospinal fluid (CSF) by the methods of this invention.

### DETAILED DESCRIPTION OF THE INVENTION

This invention provides methods for acquiring diagnostic information from biological samples which include features associated with a disease. The biological features can be closely connected to the disease pathology. More particularly, biological features of substances isolated from samples such as bodily fluids can inform of the disease and advantageously reduce the need for invasive biopsy for sampling the disease pathology.

Embodiments of this invention include methods for distinguishing extracellular matrix bodies in a biological fluid. The method allows for isolating, separating, depleting and/or enriching extracellular matrix bodies from biological fluids. More particularly, this invention discloses methods for isolating, detecting and measuring extracellular matrix bodies from a biological sample as diagnostic information and for other uses.

Disclosures of this invention include methods for obtaining diagnostic information from biological samples by studying structures isolated from components of an extracellular matrix found in a biological fluid. The structures may readily reflect components of disease pathology extant in the isolates. The structures provide information with a direct connection to the disease and are useful in diagnostic analysis.

This invention can further provide methods for obtaining biomarker information having direct association with a disease pathology. Methods disclosed herein include biomarker information with significantly enhanced level of measurement.

Embodiments of this invention include methods and compositions for separating, isolating, and/or enriching extracellular matrix bodies from bodily fluids for use as diagnostic information. The use of extracellular matrix bodies isolated and/or enriched in a biological sample can surprisingly increase diagnostic analysis for a particular biological condition or disease.

Methods and compositions of this disclosure can advantageously reduce the need for invasive patient biopsy because extracellular matrix bodies are isolated from bodily fluid samples.

This invention includes methods for preparing and isolating samples from various biological fluids which surprisingly expands the range of diagnostic information available toward particular pathologies and disease. The fractions obtained from a biological material can have strong connection to disease pathology.

Embodiments of this disclosure provide methods for preparing samples for medical information for diagnosis or prognosis of disease in a subject, by isolating extracellular matrix bodies from a biological sample from the subject, wherein the extracellular matrix bodies are associated with the disease or with a healthy subject.

In some embodiments, this invention provides methods for preparing samples for obtaining medical information, or for diagnosis, prognosis or monitoring of disease by contacting a tissue sample with a buffer or reagent to release extracellular matrix components such as extracellular matrix bodies.

In some aspects, this disclosure shows methods for obtaining extracellular matrix bodies from bodily fluids. The extracellular matrix bodies are novel structures having uses in diagnostics and development of new therapeutics, as well as for processing of bodily fluids for medical or commercial use.

In further aspects, this disclosure includes methods for separating, isolating and/or enriching extracellular matrix bodies from bodily fluids. In general, the extracellular matrix bodies cannot be observed by conventional imaging methods. This disclosure provides novel methods for isolating and detecting extracellular matrix bodies.

In additional aspects, extracellular matrix bodies of this disclosure can be surprisingly well separated from cells. Extracellular matrix bodies can also be surprisingly well separated from nano-vesicles, which are much smaller.

Methods of this invention can provide a novel window into disease pathology by separating, isolating and/or enriching extracellular matrix bodies for analysis of their properties and structure.

In general, conventional methods for analyzing a bodily fluid and isolating or purifying its components do not identify large extracellular matrix bodies as exemplified in this disclosure. Conventional methods intentionally or inadvertently discard extracellular matrix bodies.

This invention provides methods and compositions for sampling dynamic extracellular matrix structures and/or disease pathology through their presentation in bodily fluids. Extracellular matrix bodies provided by this disclosure reflect the diversity of extracellular matrix structures that determine tissue properties. Such extracellular matrix structures can be highly dynamic and constantly deposited, remodeled, and degraded to maintain tissue homeostasis. The extracellular matrix structures are spatiotemporally regulated to control cell behavior and differentiation, and dysregulation of extracellular matrix structures can lead to disease pathology.

Processes of this disclosure for separating, isolating, and/or enriching extracellular matrix bodies can be useful for identifying biomarkers of disease and therapies thereof, as well as concentrating or purifying bodily fluids.

As used herein, the term separating can include depleting and/or removing extracellular matrix bodies from a biological fluid.

Methods of this invention can provide advantageously intact biomarkers from bodily fluids.

Methods of this invention can further provide advantageously stable fractions of extracellular matrix bodies and biomarkers therefrom.

As used herein, a “biological fluid” is a “bodily fluid.”

### Extracellular Matrix Bodies

This invention discloses methods for isolating extracellular matrix bodies from a biological sample. Extracellular matrix bodies can be associated with a healthy subject or with a disease pathology in a subject, and provide markers for wellness or disease. Different bodily fluids can provide biological samples containing extracellular matrix bodies related to a biology of interest.

While not wishing to be bound by theory, the presence of extracellular matrix bodies has generally not been exploited in medicine and pharmaceutics. Conventional methods have failed to perceive, appreciate, or examine extracellular matrix bodies, in part because their presence is overwhelmed by other features, such as cells, cell components, or cell debris. Further, conventional methods intentionally or inadvertently discard extracellular matrix bodies. In the absence of cells, cell components, and/or cell debris, it has been discovered that extracellular matrix bodies can be separated, isolated, and/or enriched from a biological fluid to provide a wealth of medical information. Further, the dynamic nature of the heterogeneous structure and properties of extracellular matrix bodies has been a barrier to separating, isolating and/or enriching extracellular matrix bodies for uses in medicinal fields. Methods, compositions and discoveries described herein provide novel approaches to obtaining and utilizing extracellular matrix bodies.

As used herein, the terms extracellular matrix bodies and extracellular matrix bodies can refer to a morphologically and physiologically distinct heterogeneous mass of substances which may form a bioparticle. Extracellular matrix bodies can have various shapes with principal sizes, length or width, ranging from about 1 micrometer up to hundreds of micrometers. As used herein, a principal size can be a length, for example, a largest linear length of an object in any direction.

An extracellular matrix body bioparticle can be suspended in a bodily fluid, from which it can be separated, isolated or enriched. Extracellular matrix bodies may be composed of proteins, extracellular matrix proteins, polypeptides, lipid molecules, lipoparticles, carbohydrates, and combinations thereof. Certain components of an extracellular matrix body may be composed of nucleic acids, including any of the various forms of DNA and/or RNA. Extracellular matrix bodies may contain portions of extracellular matrix tissue structures.

The morphology of extracellular matrix bodies can range from diffuse, wherein the body may be composed of extended arms of various lengths, to a more compacted structure, wherein the body may be composed of closely-packed components; and to a more continuous structure, wherein the body may be composed of a substantially continuous mass.

The morphology of extracellular matrix bodies can be dynamic and can change with circumstances. The morphology of extracellular matrix bodies may depend on environment, such as the bodily fluid in which it is found, as well as processes to which it has been subjected, such as circulation or transport in an organism or laboratory or industrial processes. The shape and/or size of extracellular matrix bodies can vary with the environment, such as fluid temperature, pressure, flow, viscosity, ionicity, pH, osmolality, and composition.

The components of extracellular matrix bodies may remain biologically active, or biologically functional, or may have immunomodulatory properties. Extracellular matrix bodies may be formed externally to various cells, or in extracellular matrix regions of healthy or diseased tissue.

Extracellular matrix bodies present biomarkers of various kinds which can be useful as diagnostic information. Extracellular matrix bodies themselves can operate as biomarkers through their quantitative and morphological features.

The size of extracellular matrix bodies can be determined by microscopy, hydrodynamic radius, hydrodynamic volume, or radius of gyration, as well as by size fractionation methods and dynamic light scattering. The size and shape can be determined by microscopy methods. Density, mass and charge can be determined by hydrodynamic methods, light scattering methods, particle tracking methods, and electrophoretic measurements.

In some bodily fluids, extracellular matrix bodies may include various regularly-shaped microparticles or nanoparticles, typically less than about 1 micrometer in dimension, as well as irregularly shaped substances that can be attached or adhered within a body. The structures of certain components of an extracellular matrix body may include membranes, layers, or bilayers.

In certain bodily fluids, an extracellular matrix body may contain a cell, a cell from an extracellular matrix, a stromal cell, a fibroblast, an immune cell, a tumor cell, a mesenchymal cell, a vascular cell, or various other cells such as compromised or diseased cells found in bodily fluids.

In some bodily fluids, an extracellular matrix body can include within its heterogeneous structure various components such as microparticles, nanoparticles, vesicles, extracellular vesicles, exosomes, various small “mere” particles such as exomeres, endosomes, organelles, fibers, fibrous structures, and/or secretions of various cells or tissues. However, extracellular matrix bodies are in general larger than such particles and components.

In some aspects, extracellular matrix bodies isolated by the methods herein may be at least about 1 micrometer, or at least about 2 micrometers, or at least about 4 micrometers, or at least about 5 micrometers, or at least about 10 micrometers, or at least about 25 micrometers, or at least about 50 micrometers, or at least about 150 micrometers, or at least about 200 micrometers in a principal size.

In certain embodiments, extracellular matrix bodies isolated by the methods herein may be about 1 to 50 micrometers, or about 1 to 200 micrometers, or about 2 to 200 micrometers, or about 4 to 300 micrometers, or about 4 to 200 micrometers, or about 5 to 500 micrometers in a principal size.

### Isolating Extracellular Matrix Bodies

Extracellular matrix bodies are not found in cells and are not a part of cellular structure. Extracellular matrix bodies are heterogeneous bodies found in bodily fluids. In some examples, the structure of extracellular matrix bodies can be diffuse, or compacted, or a substantially continuous mass. Extracellular matrix bodies may be composed of several components, for example, various extracellular proteins, as well as certain nucleic acid molecules and various fibers or strands. Extracellular matrix bodies vary greatly in size and shape over a wide range. These features of structure can make it difficult or impossible to separate, isolate or enrich extracellular matrix bodies from a biological sample.

Further, the morphology and/or structure of extracellular matrix bodies can be dynamic and can change with experimental conditions and protocols. Embodiments of this invention show surprisingly effective methods for separating, isolating or enriching extracellular matrix bodies from a biological fluid.

Extracellular matrix bodies differ substantially from cells in density and range of sizes, shapes and structures. Embodiments of this invention include methods for separating, isolating or enriching extracellular matrix bodies by taking advantage of these differences in structure and properties. For example, cells and cell debris can be separated from extracellular matrix bodies by low speed centrifugation, and in turn, extracellular matrix bodies can be selectively separated from the remainder of a biological fluid sample by methods of this disclosure.

Embodiments of this invention provide methods for capturing and isolating at least a majority of the extracellular matrix bodies from a biological fluid. In certain embodiments, the isolate of extracellular matrix bodies can be substantially free of cells.

In further embodiments, methods of this invention can capture and isolate substantially all of the extracellular matrix bodies from a biological fluid. In certain embodiments, the isolate of extracellular matrix bodies can have an absence of cells.

Upon re-suspending an isolate of extracellular matrix bodies, the concentration of re-suspended extracellular matrix bodies can be at least 2-fold, or at least 5-fold, or at least 10-fold, or at least 100-fold enriched in concentration as compared to a biological sample, or a native biological fluid.

Examples of a biological fluid include whole blood, blood plasma, blood serum, cerebrospinal fluid, vitreous humor, aqueous humor, urine, saliva, sweat, tears, synovial fluid, pleural fluid, gastric fluid, peritoneal fluid, breast milk, nipple aspirate, ocular fluid, semen, amniotic fluid, lymph, bile, cerumen, chyle, chyme, endolymph, perilymph, exudates, feces, ejaculate, gastric acid, gastric juice, mucus, pericardial fluid, pus, rheum, sebum, serous fluid, smegma, sputum, synovial fluid, vaginal secretion, menstrual effluent, vomit and combinations thereof.

### Differential and Serial Centrifugation

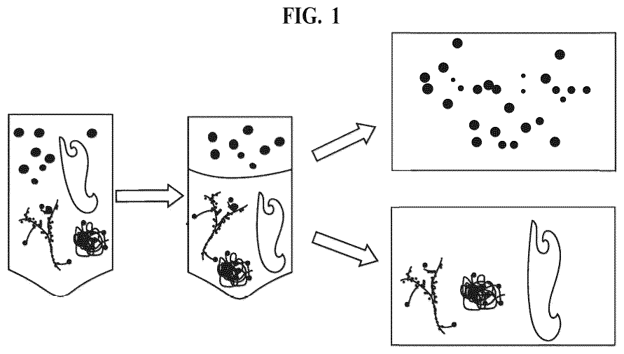

In some aspects, this disclosure provides methods for separating, isolating and/or enriching extracellular matrix bodies from bodily fluids by centrifugation. FIG. 1 shows steps to displace extracellular matrix bodies by centrifugation from a sample of bodily fluid and into a pellet form, leaving smaller biological components in the supernatant.

In various embodiments, extracellular matrix bodies can be obtained by a separation or isolation process from a bodily fluid. Some examples of methods for obtaining samples of extracellular matrix bodies from a bodily fluid include microfluidic separation, affinity chromatography, centrifugation, differential centrifugation, density gradient centrifugation, mesh filtration, diafiltration, tangential flow filtration, membrane filtration, immuno-affinity capture, magnetic bead capture, size exclusion chromatography, electrophoresis, AC electrokinetics, and combinations thereof.

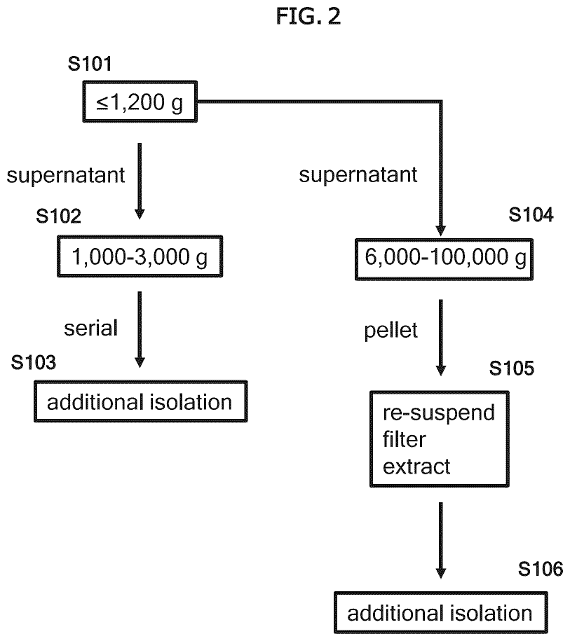

In some embodiments, this disclosure provides processes for separating, isolating or enriching extracellular matrix bodies in a pellet obtained from a biological fluid by differential centrifugation. FIG. 2 shows an initial centrifugation step S101 can be applied to the biological fluid, which applies less than about 1,200 g forces for at least about three minutes. In certain embodiments, the initial step S101 may be performed at 200 to 5,000 g for less than about ten minutes or 1-10 minutes, or at 100 to 1,200 g for less than about ten minutes or 1-10 minutes, or at 300 to 1,200 g for less than about ten minutes or 1-10 minutes. In certain embodiments, the initial step S101 may be performed at 200 to 5,000 g for about three to ten minutes. The initial step S101 can remove cells, cellular debris and other large components that are not attached to extracellular matrix bodies. Subsequently, one or more additional centrifugation steps S104 can be applied to the supernatant of the initial centrifugation, wherein at least one of the additional centrifugation steps applies about 6,000-100,000 g or greater to the supernatant. The additional steps of centrifugation can be serial centrifugation steps, in which the supernatant from a previous centrifugation step is used in the next step. The process continues with step S105 by re-suspending a final pellet obtained from the one or more additional serial centrifugation steps S104. Extracellular matrix bodies may be obtained as re-suspended in S105. The extracellular matrix bodies obtained in step S105 can be substantially free of particles smaller than about 1 micrometer, such as microparticles, nanoparticles, vesicles, extracellular vesicles, exosomes, various small “mere” particles such as exomeres, and endosomes because the centrifugation times and speeds of S101 and S104 can be kept below levels needed to force such small particles into the final pellet. In certain embodiments, step S104 may create a pellet by applying forces for times and speeds below levels needed to force small particles into the pellet, so that the pellet is substantially free of particles smaller than about 1 micrometer. The re-suspended extracellular matrix bodies may be further separated, isolated or enriched in step S106 using various methods such as a microfluidic device, additional centrifugation steps, chromatography, chemical precipitation, filtration, immuno- or affinity-capture, or a combination thereof.

In further embodiments, after removing cells and/or cell debris, any of the steps of centrifugation and filtration can be applied to a biological fluid in any order or combination in this invention. For example, after removing cells and/or cell debris, a step S104 of centrifugation to isolate extracellular matrix bodies can be preceded or followed by a step of filtration with a cut-off size where extracellular matrix bodies are extracted from the filter and re-suspended. For example, extracellular matrix bodies can be re-suspended in solution, buffer, or solvent. In another example, after removing cells and/or cell debris, a step S102 of centrifugation to isolate extracellular matrix bodies can be preceded or followed by a step of filtration with a cut-off size where extracellular matrix bodies are extracted from the filter and re-suspended.

Extracting extracellular matrix bodies may be trapped in a filter when they exceed the cut-off size. In some embodiments, extracellular matrix bodies which may be trapped in a filter can be extracted from the filter.

In some embodiments, the one or more additional centrifugation steps S104 can be applied to the supernatant of the initial centrifugation, wherein at least one of the additional centrifugation steps applies about 6,000-100,000 g, or about 6,000-80,000 g to the supernatant, or about 3,000-10,000 g, or about 3,000-20,000 g, or about 6,000-70,000 g, or about 6,000-60,000 g, or about 6,000-50,000 g, or about 6,000-40,000 g, or about 6,000-30,000 g, or about 6,000-25,000 g, or about 6,000-20,000 g. The times for additional centrifugation steps S104 to be applied can be from 1-200 minutes, or 1-100 minutes, or from 2-100 minutes, or from 3-30 minutes, or from 3-10 minutes.

In further embodiments, the one or more additional centrifugation steps S104 can be applied to the supernatant of the initial centrifugation, wherein at least one of the additional centrifugation steps applies about 6,000-80,000 g to the supernatant, or about 6,000-70,000 g, or about 6,000-60,000 g, or about 6,000-50,000 g, or about 6,000-40,000 g, or about 6,000-30,000 g, or about 6,000-25,000 g, or about 6,000-20,000 g, which steps separate substantially all of the extracellular matrix bodies.

In further embodiments, the one or more additional centrifugation steps S104 can be applied to the supernatant of the initial centrifugation, wherein at least one of the additional centrifugation steps applies about 6,000-80,000 g to the supernatant, or about 6,000-70,000 g, or about 6,000-60,000 g, or about 6,000-50,000 g, or about 6,000-40,000 g, or about 6,000-30,000 g, or about 6,000-25,000 g, or about 6,000-20,000 g, which steps separate the majority of the extracellular matrix bodies.

In further embodiments, the one or more additional centrifugation steps S104 can be applied to the supernatant of the initial centrifugation, wherein at least one of the additional centrifugation steps applies about 6,000-80,000 g to the supernatant, or about 6,000-70,000 g, or about 6,000-60,000 g, or about 6,000-50,000 g, or about 6,000-40,000 g, or about 6,000-30,000 g, or about 6,000-25,000 g, or about 6,000-20,000 g, which steps separate substantially all of the extracellular matrix bodies in the absence of cells.

In some aspects, each step of differential centrifugation may separate, isolate, or enrich a majority of the available extracellular matrix bodies. By separating, isolating, or enriching a majority of the available extracellular matrix bodies, the methods of this invention can provide surprisingly stable fractions of extracellular matrix bodies and biomarkers therefrom.

For example, a biomarker can be the quantity of one or more of, for example 1, 2, 3 or 4 of, the proteins set out in Table 1 below. For example, the biomarker may be the quantity of fibronectin.

The pellet obtained from a biological fluid by differential centrifugation may capture at least a majority of the extracellular matrix bodies from the biological fluid. In some embodiments, a pellet obtained from a biological fluid by differential centrifugation may capture at least a majority of the extracellular matrix bodies from the biological fluid with an absence of cells. In certain embodiments, a pellet obtained from a biological fluid by differential centrifugation may capture substantially all of the extracellular matrix bodies from the biological fluid. In additional embodiments, a pellet obtained from a biological fluid by differential centrifugation may capture substantially all of the extracellular matrix bodies from the biological fluid with an absence of cells.

A process of this disclosure for separating, isolating or enriching extracellular matrix bodies from a biological fluid can advantageously provide biomarker information for medical, diagnostic or prognostic use. Biomarker information can include the quantity of extracellular matrix bodies obtained from a biological fluid. Biomarker information can include the form or identity of a protein, a polypeptide, a lipid molecule, a lipoparticle, a carbohydrate, a nucleic acid molecule, or an expression level of a nucleic acid associated with extracellular matrix bodies. Biomarker information can include the form or identity of extracellular proteins or nucleic acids associated with extracellular matrix bodies.

For each step of centrifugation, the time parameter can vary from 2 to 120 minutes, or from 5 to 100 minutes, or from 10 to 50 minutes, or from 15 to 30 minutes. A centrifugation step may be applied for a time of at least 2 minutes, or at least 5 minutes, or at least 10 minutes, or at least 15 minutes. A centrifugation step may be applied for a time of less than about 2 minutes, or less than about 5 minutes, or less than about 10 minutes, or less than about 15 minutes. A centrifugation step may be carried out at any temperature, or at temperatures below ambient, or at 0-10° C., or at 4° C.

Methods of this invention for separating, isolating, and/or enriching extracellular matrix bodies can provide surprisingly intact biomarkers from bodily fluids.

Methods of this invention for separating, isolating, and/or enriching extracellular matrix bodies can provide surprisingly stable fractions of extracellular matrix bodies and biomarkers therefrom.

In some embodiments, a reagent may be added to a centrifugation step. Examples of reagents include buffers, lysing solutions, nucleic acid cleavage agents or cleavage inhibitors, precipitation agents, and fixative reagents.

The size and morphology of extracellular matrix body particles can be characterized using Zen (Zeiss) and ImageJ (NIH) software.

As used herein, the term “separated to a high degree” can refer to a high degree as being a majority, or at least about 60%.

In some aspects, this disclosure provides methods for separating, isolating and/or enriching extracellular matrix bodies from bodily fluids by serial centrifugation.

In various embodiments, extracellular matrix bodies can be obtained by a separation or isolation process from a bodily fluid. Some examples of methods for obtaining samples of extracellular matrix bodies from a bodily fluid include microfluidic separation, affinity chromatography, centrifugation, differential centrifugation, density gradient centrifugation, mesh filtration, diafiltration, tangential flow filtration, membrane filtration, immuno-affinity capture, magnetic bead capture, size exclusion chromatography, electrophoresis, and combinations thereof.

In some embodiments, this disclosure provides processes for separating, isolating or enriching extracellular matrix bodies in a pellet obtained from a biological fluid by serial centrifugation. FIG. 2 shows an initial centrifugation step S101 can be applied to the biological fluid, which applies less than about 1,200 g forces for at least about two minutes. The initial step S101 can apply forces for a period of time sufficient to remove from the supernatant cells, cell debris, and other large components that are not attached to extracellular matrix bodies. In certain embodiments, the initial step S101 may be performed at 200 to 5,000 g for about three to ten minutes. Subsequently, one or more additional centrifugation steps S102 can be applied to the supernatant of the initial centrifugation, wherein at least one of the additional centrifugation steps each apply about 1,000-3,000 g to the supernatant. The additional steps of centrifugation can be serial centrifugation steps, in which the supernatant from a previous centrifugation step is used in the next step. Extracellular matrix bodies in the final supernatant from steps S102 of serial centrifugation can be greatly enriched in concentration as compared to the biological fluid, and can be substantially free of cells or cell debris. The final supernatant from serial centrifugation may be further separated, isolated or enriched in step S103 using various methods such as a microfluidic device, additional centrifugation steps, chromatography, chemical precipitation, electrophoresis, AC electrokinetics, filtration, immuno- or affinity-capture, or a combination thereof. The supernatant from serial centrifugation may contain relatively fewer extracellular matrix bodies as compared to the pellet of these steps, however, the serial centrifugation may advantageously provide a collection of stable extracellular matrix bodies, or a collection of extracellular matrix bodies of more uniform morphology.

In further embodiments, after removing cells and/or cell debris, any of the steps of centrifugation and filtration can be applied to a biological fluid in any order or combination in this invention. In another example, after removing cells and/or cell debris, a step S102 of centrifugation to isolate extracellular matrix bodies can be preceded or followed by a step of filtration with a cut-off size where extracellular matrix bodies are extracted from the filter and re-suspended. The cut-off size can be made small enough to pass through small particles, but trap large particles such as extracellular matrix bodies in the filter. By extracting the extracellular matrix bodies from the filter and re-suspending, the resulting suspension can be substantially free of small particles such as exosomes.

In certain embodiments, the initial centrifugation step S101 may be performed at about 200 to about 10,000 g, or at about 200 to about 5,000 g, or 400 to 8,000 g, or 500 to 7,000 g, or 600 to 6,000 g, or 800 to 5,000 g, or 1,000 to 4,000 g, or 1,200 to 3,000 g. The times for serial centrifugation steps S101 to be applied can be from 1-30 minutes, or from 1-10 minutes, or from 2-10 minutes, or from 3-10 minutes.

In some embodiments, the additional serial centrifugation steps S102 may each apply about 300-3,000 g, 1,000-3,000 g, 1,000-6,000 g, or 1,000-12,000 g, or 1,000-20,000 g, or 1,000-25,000 g, or 1,000-30,000 g, or 1,000-40,000 g, or 1,000-50,000 g, or 1,000-60,000 g, or greater. The times for serial centrifugation steps S102 to be applied can be from 1-100 minutes, or from 1-30 minutes, or from 1-10 minutes, or from 2-10 minutes, or from 3-10 minutes.

In certain embodiments, the additional serial centrifugation steps S102 may each apply the same force. For example, each additional serial centrifugation step may apply 3,000 g. Each additional serial centrifugation step may be applied for the same amount of time, or for a different amount of time.

The supernatant obtained from a biological fluid by serial centrifugation may capture at least a majority of the extracellular matrix bodies from the biological fluid. In some embodiments, supernatant obtained from a biological fluid by serial centrifugation may capture at least a majority of the extracellular matrix bodies from the biological fluid with an absence of cells. In certain embodiments, supernatant obtained from a biological fluid by serial centrifugation may capture substantially all of the extracellular matrix bodies from the biological fluid. In additional embodiments, supernatant obtained from a biological fluid by serial centrifugation may capture substantially all of the extracellular matrix bodies from the biological fluid with an absence of cells.

The supernatant obtained from a biological fluid by serial centrifugation may capture and enrich a specific portion of the extracellular matrix bodies from the biological fluid, so that extracellular matrix bodies of the specific portion can be distinguished from particles smaller than about 1 micrometer, such as microparticles, nanoparticles, vesicles, extracellular vesicles, exosomes, various small “mere” particles such as exomeres, and endosomes. The specific portion of the extracellular matrix bodies can be selected by adjusting the speeds and times of serial centrifugation steps S102. Methods based on steps S101, S102, and S103 can greatly enrich extracellular matrix bodies of a specific portion in a supernatant relative to particles smaller than about 1 micrometer, such as microparticles, nanoparticles, vesicles, extracellular vesicles, exosomes, various small “mere” particles such as exomeres, and endosomes.

In certain aspects, a step of serial centrifugation may separate, isolate, or enrich a stable fraction of the available extracellular matrix bodies. By separating, isolating, or enriching a stable fraction of the available extracellular matrix bodies, the methods of this invention can provide surprisingly intact biomarkers from bodily fluids. In further aspects, a step of serial centrifugation may separate, isolate, or enrich a stable fraction of the available extracellular matrix bodies, which includes intact bodies having diffuse morphology that are preserved.

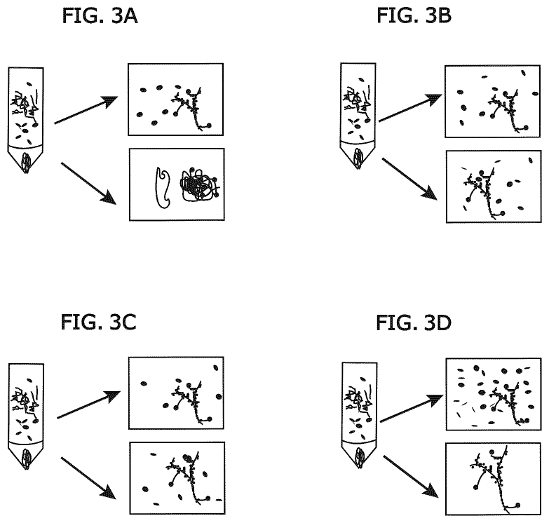

FIG. 3A shows that some extracellular matrix bodies can be separated, isolated, or enriched by initial centrifugation of a sample of bodily fluid. FIG. 3B shows that by using a serial step of centrifugation, using supernatant from a previous step, some extracellular matrix bodies can be separated, isolated, or enriched in a sample of bodily fluid. FIG. 3C and FIG. 3D show that by using one or more additional serial steps of centrifugation, each using supernatant from a previous step, some extracellular matrix bodies can be separated, isolated, or enriched in a sample of bodily fluid.

A process of this disclosure for separating, isolating or enriching extracellular matrix bodies from a biological fluid can advantageously provide biomarker information for medical, diagnostic or prognostic use. Biomarker information can include the quantity of extracellular matrix bodies obtained from a biological fluid. Biomarker information can include the form or identity of a protein, a polypeptide, a lipid molecule, a lipoparticle, a carbohydrate, a nucleic acid molecule, or an expression level of a nucleic acid associated with extracellular matrix bodies. Biomarker information can include the form or identity of extracellular proteins or nucleic acids associated with extracellular matrix bodies.

For each step of centrifugation, the time parameter can vary from 2 to 120 minutes, or from 5 to 100 minutes, or from 10 to 50 minutes, or from 15 to 30 minutes. A centrifugation step may be applied for a time of at least 2 minutes, or at least 5 minutes, or at least 10 minutes, or at least 15 minutes. A centrifugation step may be applied for a time of less than about 2 minutes, or less than about 5 minutes, or less than about 10 minutes, or less than about 15 minutes. A centrifugation step may be carried out at any temperature, or at temperatures below ambient, or at 0-10° C., or at 4° C.

In some embodiments, a reagent may be added to a centrifugation step. Examples of reagents include buffers, lysing solutions, nucleic acid cleavage agents or cleavage inhibitors, precipitation agents, and fixative reagents.

In some embodiments, when a biological fluid is centrifuged, it may preferably be centrifuged by differential centrifugation or serial centrifugation, or a combination thereof. When differential centrifugation is performed, it preferably applies from about 4,000 to about 15,000 g, more preferably at from about 6,000 to about 12,000 g.

In some embodiments, when a biological fluid is filtered, it may preferably be filtered by single pass membrane filtration or multiple pass serial membrane filtration. For example, a cellulose acetate membrane filter may be used with a pore size of from about 0.2 to about 1 micron, or from about 0.45 micron to about 4 micron. Extracellular matrix bodies may be retained (trapped) in the filter when a principal size is larger than the pore size.

A process for separating, isolating or enriching extracellular matrix bodies in a biological fluid may comprise both centrifuging and filtering the biological fluid.

### Density Gradient Centrifugation

This disclosure provides methods for separating, isolating and/or enriching extracellular matrix bodies from bodily fluids by density gradient centrifugation. Some methods for centrifugation are described in C. A. Price, Centrifugation in Density Gradients (1982 Elsevier).

In some embodiments, extracellular matrix bodies can be obtained by a separation or isolation process from a bodily fluid. Some examples of methods for obtaining samples of extracellular matrix bodies from a bodily fluid include microfluidic separation, affinity chromatography, centrifugation, differential centrifugation, density gradient centrifugation, mesh filtration, diafiltration, tangential flow filtration, membrane filtration, immuno-affinity capture, magnetic bead capture, size exclusion chromatography, electrophoresis, AC electrokinetics, and combinations thereof.

Methods of this invention for separating, isolating, and/or enriching extracellular matrix bodies can provide surprisingly intact biomarkers from bodily fluids.

Methods of this invention for separating, isolating, and/or enriching extracellular matrix bodies can provide surprisingly stable fractions of extracellular matrix bodies and biomarkers therefrom.

### Membrane Filtration

This disclosure provides methods for separating, isolating and/or enriching extracellular matrix bodies from bodily fluids by membrane filtration.

In some embodiments, extracellular matrix bodies can be obtained by a separation or isolation process from a bodily fluid. Some examples of methods for obtaining samples of extracellular matrix bodies from a bodily fluid include microfluidic separation, affinity chromatography, centrifugation, differential centrifugation, density gradient centrifugation, mesh filtration, diafiltration, tangential flow filtration, membrane filtration, immuno-affinity capture, magnetic bead capture, size exclusion chromatography, electrophoresis, AC electrokinetics, and combinations thereof.

In any process of membrane filtration, as used herein, and as known in the art, a precipitate, a residue, an extract, or an isolate can be obtained from the feed or entry or proximal side of a membrane, by reverse flow of fluid through the filter. A filtrate can be obtained from the distal side of the membrane, in general, by forward flow of fluid through the filter.

In some embodiments, filtering of a biological fluid can be performed with a filter for passing particles of less than a cutoff size, wherein the cutoff size is 1, or 2, or 3, or 4, or 5, or 6, or 10 micrometers, and extracellular matrix bodies are extracted from the filter.

In filtration, as used herein, and as known in the art, a membrane may also be referred to as a frit, or a disc, or a frit membrane, or a sheet, or a screen, any of which can be barriers to flow having fenestrations, pore sizes, and/or cutoff sizes.

Filtration methods of this invention for separating, isolating, and/or enriching extracellular matrix bodies can provide surprisingly intact biomarkers from bodily fluids.

Filtration methods of this invention for separating, isolating, and/or enriching extracellular matrix bodies can provide surprisingly stable fractions of extracellular matrix bodies and biomarkers therefrom.

Filtration methods of this invention for separating, isolating, and/or enriching extracellular matrix bodies include centrifugal filtration methods.

### Ultrafiltration

This disclosure provides methods for separating, isolating and/or enriching extracellular matrix bodies from bodily fluids by ultrafiltration. Some methods for ultrafiltration are described in M. Cheryan, Ultrafiltration Handbook (1997 CRC Press), and K. Scott, Handbook of Industrial Membranes (2^{nd} Ed 1995 Elsevier).

In some embodiments, extracellular matrix bodies can be obtained by a separation or isolation process from a bodily fluid. Some examples of methods for obtaining samples of extracellular matrix bodies from a bodily fluid include microfluidic separation, affinity chromatography, centrifugation, differential centrifugation, density gradient centrifugation, mesh filtration, diafiltration, tangential flow filtration, membrane filtration, immuno-affinity capture, magnetic bead capture, size exclusion chromatography, electrophoresis, AC electrokinetics, and combinations thereof.

### Flow Filtration

This disclosure provides methods for separating, isolating and/or enriching extracellular matrix bodies from bodily fluids by flow filtration. Some methods for flow filtration are described in G. Jagschies et al., Biopharmaceutical Processing (2017 Elsevier).

In some embodiments, extracellular matrix bodies can be obtained by a separation or isolation process from a bodily fluid. Some examples of methods for obtaining samples of extracellular matrix bodies from a bodily fluid include microfluidic separation, affinity chromatography, centrifugation, differential centrifugation, density gradient centrifugation, mesh filtration, diafiltration, tangential flow filtration, membrane filtration, immuno-affinity capture, magnetic bead capture, size exclusion chromatography, electrophoresis, AC electrokinetics, and combinations thereof.

### Extracellular Matrix Bodies Presenting Biomarkers

This invention can further provide methods for obtaining biomarker information having direct association with a disease pathology. Methods disclosed herein include biomarker information with significantly enhanced level of measurement.

Extracellular matrix bodies present biomarkers of various kinds which can be useful as diagnostic information. Extracellular matrix bodies themselves can operate as biomarkers through their quantitative and morphological features.

A process of this disclosure for separating, isolating or enriching extracellular matrix bodies from a biological fluid can advantageously provide biomarker information for medical, diagnostic or prognostic use. Biomarker information can include the quantity of extracellular matrix bodies obtained from a biological fluid. Biomarker information can include the form or identity of a protein, a polypeptide, a lipid molecule, a lipoparticle, a carbohydrate, a nucleic acid molecule, or an expression level of a nucleic acid associated with extracellular matrix bodies. Biomarker information can include the form or identity of extracellular proteins or nucleic acids associated with extracellular matrix bodies.

Some examples of biomarkers found in extracellular matrix bodies include proteins given in Table 1.

TABLE 1
Biomarkers found in extracellular matrix bodies

| No. | GENE | IDENTIFIER |
|---|---|---|
| 1. | IGHG4 | tr\|A0A286YFJ8\|A0A286YFJ8_HUMAN |
| 2. | PIGR | sp\|P01833\|PIGR_HUMAN |
| 3. | IGHA1 | sp\|P01876\|IGHA1_HUMAN |
| 4. | IGLV4-60 | sp\|A0A075B6I1\|LV460_HUMAN |
| 5. | IGHD | sp\|P01880\|IGHD_HUMAN |
| 6. | HBB | sp\|P68871\|HBB_HUMAN |
| 7. | HPR | sp\|P00739\|HPTR_HUMAN |
| 8. | IGKV1-6 | sp\|A0A0C4DH72\|KV106_HUMAN |
| 9. | IGHM | sp\|P01871\|IGHM_HUMAN |
| 10. | HBA1 | sp\|P69905\|HBA_HUMAN |
| 11. | CFB | tr\|A0A0G2JH38\|A0A0G2JH38_HUMAN |
| 12. | LPA | sp\|P08519\|APOA_HUMAN |
| 13. | F12 | sp\|P00748\|FA12_HUMAN |
| 14. | B4E1Z4 | tr\|B4E1Z4\|B4E1Z4_HUMAN |
| | | (C3/C5 convertase) |
| 15. | IGHV3-33 | sp\|P01772\|HV333_HUMAN |
| 16. | SERPINF2 | sp\|P08697\|A2AP_HUMAN |
| 17. | IGHV2-70D | sp\|A0A0C4DH43\|HV70D_HUMAN |
| 18. | JCHAIN | sp\|P01591\|IGJ_HUMAN |
| 19. | IGHV6-1 | sp\|A0A0B4J1U7\|HV601_HUMAN |
| 20. | TLN1 | sp\|Q9Y490\|TLN1_HUMAN |
| 21. | FGA | sp\|P02671\|FIBA_HUMAN |
| 22. | THBS1 | sp\|P07996\|TSP1_HUMAN |
| 23. | IGHA2 | tr\|A0A286YEY5\|A0A286YEY5_HUMAN |
| 24. | CFH | tr\|A0A0D9SG88\|A0A0D9SG88_HUMAN |
| 25. | IGHV5-51 | sp\|A0A0C4DH38\|HV551_HUMAN |
| 26. | IGHV4-39 | sp\|P01824\|HV439_HUMAN |
| 27. | IGKV3-20 | sp\|P01619\|KV320_HUMAN |
| 28. | IGHV3-53 | sp\|P01767\|HV353_HUMAN |
| 29. | ITIH1 | sp\|P19827\|ITIH1_HUMAN |
| 30. | A2M | sp\|P01023\|A2MG_HUMAN |
| 31. | CFD | tr\|K7ERG9\|K7ERG9_HUMAN |
| 32. | CRP | sp\|P02741\|CRP_HUMAN |
| 33. | CD5L | sp\|O43866\|CD5L_HUMAN |
| 34. | LGALS3BP | sp\|Q08380\|LG3BP_HUMAN |
| 35. | IGKV3-15 | sp\|P01624\|KV315_HUMAN |
| 36. | PON1 | sp\|P27169\|PON1_HUMAN |
| 37. | ORM1 | sp\|P02763\|A1AG1_HUMAN |
| 38. | SLC4A1 | sp\|P02730\|B3AT_HUMAN |
| 39. | APOC4-APOC2 | tr\|K7ER74\|K7ER74_HUMAN |
| 40. | IGKV2-29 | sp\|A2NJV5\|KV229_HUMAN |
| 41. | FGB | sp\|P02675\|FIBB_HUMAN |
| 42. | IGLC2 | sp\|P0DOY2\|IGLC2_HUMAN |
| 43. | IGHV3OR15-7 | tr\|A0A075B7D8\|A0A075B7D8_HUMAN |
| 44. | IGHA2 | tr\|A0A0G2JMB2\|A0A0G2JMB2_HUMAN |
| 45. | IGLV8-61 | sp\|A0A075B610\|LV861_HUMAN |
| 46. | IGHV3-21 | sp\|A0A0B4J1V1\|HV321_HUMAN |
| 47. | IGLV4-69 | sp\|A0A075B6H9\|LV469_HUMAN |
| 48. | IGLV1-44 | sp\|P01699\|LV144_HUMAN |
| 49. | C4B | sp\|P0C0L5\|CO4B_HUMAN |
| 50. | C7 | sp\|P10643\|CO7_HUMAN |
| 51. | ACTB | sp\|P60709\|ACTB_HUMAN |
| 52. | IGLV3-21 | sp\|P80748\|LV321_HUMAN |
| 53. | PGLYRP2 | sp\|Q96PD5\|PGRP2_HUMAN |
| 54. | IGHV1-18 | sp\|A0A0C4DH31\|HV118_HUMAN |
| 55. | IGKV2D-28 | tr\|A0A5H1ZRS2\|A0A5H1ZRS2_HUMAN |
| | | (P01615) |
| 56. | IGKV2-24 | sp\|A0A0C4DH68\|KV224_HUMAN |
| 57. | FBLL1 | tr\|A0A0G2JRQ6\|A0A0G2JRQ6_HUMAN |
| | | (fibrillarin) |
| 58. | IGKC | sp\|P01834\|IGKC_HUMAN |
| 59. | IGHV3-9 | sp\|P01782\|HV309_HUMAN |
| 60. | ITIH4 | sp\|Q14624\|ITIH4_HUMAN |
| 61. | AHSG | sp\|P02765\|FETUA_HUMAN |
| 62. | AGT | sp\|P01019\|ANGT_HUMAN |
| 63. | IGHV1-69 | sp\|P01742\|HV169_HUMAN |
| 64. | IGHV1-3 | sp\|A0A0C4DH29\|HV103_HUMAN |
| 65. | SPTA1 | sp\|P02549\|SPTA1_HUMAN |
| 66. | IGLV2-11 | sp\|P01706\|LV211_HUMAN |
| 67. | IGKV1D-33 | tr\|A0A2Q2TTZ9\|A0A2Q2TTZ9_HUMAN |
| 68. | LRG1 | sp\|P02750\|A2GL_HUMAN |
| 69. | IGHV3-72 | tr\|A0A4W8ZXM2\|A0A4W8ZXM2_HUMAN |
| 70. | FGG | sp\|P02679\|FIBG_HUMAN |
| 71. | APOC1 | tr\|K7ERI9\|K7ERI9_HUMAN |
| 72. | LYZ | sp\|P61626\|LYSC_HUMAN |
| 73. | IGKV1-5 | sp\|P01602\|KV105_HUMAN |
| 74. | IGKV1-27 | sp\|A0A075B6S5\|KV127_HUMAN |
| 75. | IGHV1-46 | sp\|P01743\|HV146_HUMAN |
| 76. | C1R | tr\|A0A3B3ISR2\|A0A3B3ISR2_HUMAN |
| 77. | IGKV1-16 | sp\|P04430\|KV116_HUMAN |
| 78. | C4BPB | sp\|P20851-2\|C4BPB_HUMAN |
| 79. | IGFALS | sp\|P35858\|ALS_HUMAN |
| 80. | IGKV4-1 | sp\|P06312\|KV401_HUMAN |
| 81. | PF4 | sp\|P02776\|PLF4_HUMAN |
| 82. | IGHV1OR15-1 | tr\|A0A075B7D0\|A0A075B7D0_HUMAN |
| 83. | IGKV1D-12 | sp\|P01611\|KVD12_HUMAN |
| 84. | IGKV2-30 | sp\|P06310\|KV230_HUMAN |
| 85. | IGHV3-74 | sp\|A0A0B4J1X5\|HV374_HUMAN |
| 86. | SERPING1 | sp\|P05155\|IC1_HUMAN |
| 87. | IGHV3-43D | sp\|P0DP04\|HV43D_HUMAN |
| 88. | APOH | sp\|P02749\|APOH_HUMAN |
| 89. | C1S | sp\|P09871\|C1S_HUMAN |
| 90. | PLG | sp\|P00747\|PLMN_HUMAN |
| 91. | IGLV3-9 | sp\|A0A075B6K5\|LV39_HUMAN |
| 92. | IGHV3-49 | sp\|A0A0A0MS15\|HV349_HUMAN |
| 93. | APOL1 | sp\|O14791\|APOL1_HUMAN |
| 94. | SERPINC1 | sp\|P01008\|ANT3_HUMAN |
| 95. | CFP | sp\|P27918\|PROP_HUMAN |
| 96. | AMBP | sp\|P02760\|AMBP_HUMAN |
| 97. | C6 | sp\|P13671\|CO6_HUMAN |
| 98. | IGHV3-38 | sp\|A0A0C4DH36\|HV338_HUMAN |
| 99. | IGKV2D-29 | tr\|A0A5H1ZRS9\|A0A5H1ZRS9_HUMAN |
| 100. | IGHV4-28 | sp\|A0A0C4DH34\|HV428_HUMAN |
| 101. | ADIPOQ | sp\|Q15848\|ADIPO_HUMAN |
| 102. | IGHV3-73 | sp\|A0A0B4J1V6\|HV373_HUMAN |
| 103. | IGHG3 | tr\|A0A286YES1\|A0A286YES1_HUMAN |
| 104. | CPN1 | sp\|P15169\|CBPN_HUMAN |
| 105. | PROS1 | tr\|A0A3B3ISJ1\|A0A3B3ISJ1_HUMAN |
| 106. | IGHG2 | tr\|A0A286YEY4\|A0A286YEY4_HUMAN |
| 107. | IGHV7-4-1 | sp\|A0A0J9YVY3\|HV741_HUMAN |
| 108. | F2 | sp\|P00734\|THRB_HUMAN |
| 109. | IGLV3-10 | sp\|A0A075B6K4\|LV310_HUMAN |
| 110. | IGLV6-57 | sp\|P01721\|LV657_HUMAN |
| 111. | CFH | sp\|P08603\|CFAH_HUMAN |
| 112. | ALB | sp\|P02768\|ALBU_HUMAN |
| 113. | HRG | sp\|P04196\|HRG_HUMAN |
| 114. | FN1 | sp\|P02751-8\|FINC_HUMAN fibronectin |
| 115. | C3 | sp\|P01024\|CO3_HUMAN |
| 116. | C1QB | tr\|D6R934\|D6R934_HUMAN |
| 117. | F5 | tr\|A0A0A0MRJ7\|A0A0A0MRJ7_HUMAN |
| 118. | GC | sp\|P02774\|VTDB_HUMAN |
| 119. | IGLV1-51 | sp\|P01701\|LV151_HUMAN |
| 120. | PLTP | sp\|P55058\|PLTP_HUMAN |
| 121. | EFEMP1 | sp\|Q12805\|FBLN3_HUMAN |
| 122. | SERPINA5 | sp\|P05154\|IPSP_HUMAN |
| 123. | CPN2 | sp\|P22792\|CPN2_HUMAN |
| 124. | IGKV1-13 | sp\|P0DP09\|KV113_HUMAN |
| 125. | IGKV3-11 | sp\|P04433\|KV311_HUMAN |
| 126. | IGLV3-25 | sp\|P01717\|LV325_HUMAN |
| 127. | SHBG | tr\|I3L145\|I3L145_HUMAN |
| 128. | FCGBP | tr\|A0A087WXI2\|A0A087WXI2_HUMAN |
| 129. | AZGP1 | sp\|P25311\|ZA2G_HUMAN |
| 130. | ING4 | tr\|E9PNE3\|E9PNE3_HUMAN |
| 131. | APOD | tr\|C9JF17\|C9JF17_HUMAN |
| 132. | HPX | sp\|P02790\|HEMO_HUMAN |
| 133. | CD14 | sp\|P08571\|CD14_HUMAN |
| 134. | APOE | sp\|P02649\|APOE_HUMAN |
| 135. | IGLV3-19 | sp\|P01714\|LV319_HUMAN |
| 136. | C9 | sp\|P02748\|CO9_HUMAN |
| 137. | APOM | sp\|O95445\|APOM_HUMAN |
| 138. | IGKV3-7 | sp\|A0A075B6H7\|KV37_HUMAN |
| 139. | IGLL5 | tr\|A0A0B4J231\|A0A0B4J231_HUMAN |
| 140. | ITIH2 | sp\|P19823\|ITIH2_HUMAN |
| 141. | IGKV1-9 | sp\|A0A0C4DH69\|KV109_HUMAN |
| 142. | ECM1 | sp\|Q16610\|ECM1_HUMAN |
| 143. | IGHV3OR16-12 | tr\|A0A075B7B8\|A0A075B7B8_HUMAN |
| 144. | CLEC3B | tr\|E9PHK0\|E9PHK0_HUMAN |
| 145. | APOB | sp\|P04114\|APOB_HUMAN |
| 146. | CFHR2 | tr\|A0A3B3IQ51\|A0A3B3IQ51_HUMAN |
| 147. | IG-unk | tr\|A0A0J9YY99\|A0A0J9YY99_HUMAN |
| 148. | IGKV1D-39 | sp\|P04432\|KVD39_HUMAN |
| 149. | TTR | sp\|P02766\|TTHY_HUMAN |
| 150. | FBLN1 | sp\|P23142\|FBLN1_HUMAN |
| 151. | IGHV3-64 | sp\|A0A075B6Q5\|HV364_HUMAN |
| 152. | KLKB1 | tr\|H0YAC1\|H0YAC1_HUMAN |
| 153. | LUM | sp\|P51884\|LUM_HUMAN |
| 154. | VTN | sp\|P04004\|VTNC_HUMAN |
| 155. | C5 | sp\|P01031\|CO5_HUMAN |
| 156. | PZP | sp\|P20742\|PZP_HUMAN |
| 157. | SERPIND1 | sp\|P05546\|HEP2_HUMAN |
| 158. | F13B | sp\|P05160\|F13B_HUMAN |
| 159. | HABP2 | sp\|Q14520\|HABP2_HUMAN |
| 160. | APOA2 | tr\|V9GYM3\|V9GYM3_HUMAN |
| 161. | SERPINA6 | sp\|P08185\|CBG_HUMAN |
| 162. | IGLL1 | sp\|P15814\|IGLL1_HUMAN |
| 163. | APOA4 | sp\|P06727\|APOA4_HUMAN |
| 164. | IGHV3-66 | sp\|A0A0C4DH42\|HV366_HUMAN |
| 165. | IGKV6D-21 | sp\|A0A0A0MT36\|KVD21_HUMAN |
| 166. | IGHV3-13 | sp\|P01766\|HV313_HUMAN |
| 167. | ALB | tr\|A0A0C4DGB6\|A0A0C4DGB6_HUMAN |
| 168. | IGKV1D-37 | sp\|P0DSN7\|KVD37_HUMAN |
| 169. | IGKV1-17 | sp\|P01599\|KV117_HUMAN |
| 170. | ITIH3 | sp\|Q06033\|ITIH3_HUMAN |
| 171. | APCS | sp\|P02743\|SAMP_HUMAN |
| 172. | CPB2 | sp\|Q96IY4\|CBPB2_HUMAN |
| 173. | CFI | tr\|E7ETH0\|E7ETH0_HUMAN |
| 174. | C1QC | sp\|P02747\|C1QC_HUMAN |
| 175. | SERPINF1 | sp\|P36955\|PEDF_HUMAN |
| 176. | TGFBI | sp\|Q15582\|BGH3_HUMAN |
| 177. | C8G | sp\|P07360\|CO8G_HUMAN |
| 178. | IGHV2-5 | sp\|P01817\|HV205_HUMAN |
| 179. | APOA1 | sp\|P02647\|APOA1_HUMAN |
| 180. | F9 | sp\|P00740\|FA9_HUMAN |
| 181. | CLU | sp\|P10909-6\|CLUS_HUMAN |
| 182. | KNG1 | sp\|P01042-2\|KNG1_HUMAN |
| 183. | IGLV1-40 | sp\|P01703\|LV140_HUMAN |
| 184. | SERPINA1 | sp\|P01009\|A1AT_HUMAN |
| 185. | C8A | sp\|P07357\|CO8A_HUMAN |
| 186. | IGHG1 | tr\|A0A0A0MS08\|A0A0A0MS08_HUMAN |
| 187. | SELENOP | tr\|A0A182DWH7\|A0A182DWH7_HUMAN |
| 188. | HP | sp\|P00738\|HPT_HUMAN |
| 189. | IGHV4-38-2 | sp\|P0DP08\|HVD82_HUMAN |
| 190. | SERPINA4 | sp\|P29622\|KAIN_HUMAN |
| 191. | LBP | sp\|P18428\|LBP_HUMAN |
| 192. | ORM2 | sp\|P19652\|A1AG2_HUMAN |
| 193. | FN1 | sp\|P02751-10\|FINC_HUMAN |
| 194. | F10 | sp\|P00742\|FA10_HUMAN |
| 195. | APOC3 | tr\|B0YIW2\|B0YIW2_HUMAN |
| 196. | CFHR1 | sp\|Q03591\|FHR1_HUMAN |
| 197. | ATRN | sp\|O75882\|ATRN_HUMAN |
| 198. | B2M | sp\|P61769\|B2MG_HUMAN |
| 199. | VWF | sp\|P04275\|VWF_HUMAN |
| 200. | F13A1 | sp\|P00488\|F13A_HUMAN |
| 201. | AFM | sp\|P43652\|AFAM_HUMAN |
| 202. | GPX3 | tr\|A0A087X1J7\|A0A087X1J7_HUMAN |
| 203. | FCN3 | sp\|O75636\|FCN3_HUMAN |
| 204. | RBP4 | tr\|Q5VY30\|Q5VY30_HUMAN |
| 205. | C8B | sp\|P07358\|CO8B_HUMAN |
| 206. | SERPINA3 | sp\|P01011\|AACT_HUMAN |
| 207. | PRG4 | sp\|Q92954\|PRG4_HUMAN |
| 208. | C4A | sp\|P0C0L4\|CO4A_HUMAN |
| 209. | SAA2-SAA4 | tr\|A0A096LPE2\|A0A096LPE2_HUMAN |
| 210. | SERPINA7 | sp\|P05543\|THBG_HUMAN |
| 211. | CP | sp\|P00450\|CERU_HUMAN |
| 212. | A1BG | sp\|P04217\|A1BG_HUMAN |
| 213. | C2 | sp\|P06681\|CO2_HUMAN |
| 214. | IGHV1-2 | sp\|P23083\|HV102_HUMAN |
| 215. | HGFAC | tr\|D6RAR4\|D6RAR4_HUMAN |
| 216. | FETUB | sp\|Q9UGM5\|FETUB_HUMAN |
| 217. | GSN | sp\|P06396\|GELS_HUMAN |
| 218. | SERPINA1 | tr\|A0A024R6I7\|A0A024R6I7_HUMAN |
| 219. | C1QA | sp\|P02745\|C1QA_HUMAN |
| 220. | C4BPA | sp\|P04003\|C4BPA_HUMAN |
| 221. | IGKV3OR2-268 | tr\|A0A0C4DH90\|A0A0C4DH90_HUMAN |
| 222. | HSPG2 | B6EU51 (B6EU51_HUMAN) |
| 223. | FGG | P02679 (FIBG_HUMAN) |
| 224. | FGA | P02671 (FIBA_HUMAN) |
| 225. | FGB | P02675 (FIBB_HUMAN) |
| 226. | APOC1 | P02654 (APOC1_HUMAN) |
| 227. | LYZ | P61626 (LYSC_HUMAN) |
| 228. | ANG | P03950 (ANGI_HUMAN) |
| 229. | IGFBP5 | P24593 (IBP5_HUMAN) |
| 230. | C1QB | P02746 (C1QB_HUMAN) |
| 231. | F5 | P12259 (FA5_HUMAN) |
| 232. | H1-4 | P10412 (H14_HUMAN) |
| 233. | HBA1 | P69905 (HBA_HUMAN) |
| 234. | KRT10 | P13645 (K1C10_HUMAN) |
| 235. | H1-0 | P07305 (H10_HUMAN) |
| 236. | QSOX1 | O00391 (QSOX1_HUMAN) |
| 237. | HABP2 | Q14520 (HABP2_HUMAN) |
| 238. | KRT9 | P35527 (K1C9_HUMAN) |
| 239. | KRT2 | P35908 (K22E_HUMAN) |
| 240. | VTN | P04004 (VTNC_HUMAN) |
| 241. | H4C1 | P62805 (H4_HUMAN) |
| 242. | KRT1 | P04264 (K2C1_HUMAN) |
| 243. | C8B | P07358 (CO8B_HUMAN) |
| 244. | C1QA | P02745 (C1QA_HUMAN) |
| 245. | MTHFD2 | P13995 (MTDC_HUMAN) |
| 246. | CFHR1 | Q03591 (FHR1_HUMAN) |
| 247. | ACTB | P60709 (ACTB_HUMAN) |
| 248. | HRG | P04196 (HRG_HUMAN) |
| 249. | EEF1A1 | P68104 (EF1A1_HUMAN) |
| 250. | H2AC4 | P04908 (H2A1B_HUMAN) |
| 251. | MMP2 | P08253 (MMP2_HUMAN) |
| 252. | TIMP2 | P16035 (TIMP2_HUMAN) |
| 253. | ATP5F1B | P06576 (ATPB_HUMAN) |
| 254. | HGFAC | Q04756 (HGFA_HUMAN) |
| 255. | ENO1 | P06733 (ENOA_HUMAN) |
| 256. | HSPG2 | P98160 (PGBM_HUMAN) |
| 257. | COL18A1 | P39060 (COIA1_HUMAN) |
| 258. | APOE | P02649 (APOE_HUMAN) |
| 259. | CFH | P08603 (CFAH_HUMAN) |
| 260. | RARRES2 | Q99969 (RARR2_HUMAN) |
| 261. | SERPINA5 | P05154 (IPSP_HUMAN) |
| 262. | SAA4 | P35542 (SAA4_HUMAN) |
| 263. | PEBP4 | Q96S96 (PEBP4_HUMAN) |
| 264. | C1QC | P02747 (C1QC_HUMAN) |
| 265. | CHI3L1 | P36222 (CH3L1_HUMAN) |
| 266. | PON1 | P27169 (PON1_HUMAN) |
| 267. | CLU | P10909 (CLUS_HUMAN) |
| 268. | SERPIND1 | P05546 (HEP2_HUMAN) |
| 269. | PCOLCE | Q15113 (PCOC1_HUMAN) |
| 270. | C8A | P07357 (CO8A_HUMAN) |
| 271. | APOM | O95445 (APOM_HUMAN) |
| 272. | APOA1 | P02647 (APOA1_HUMAN) |
| 273. | APOD | P05090 (APOD_HUMAN) |
| 274. | ECM1 | Q16610 (ECM1_HUMAN) |
| 275. | SELENOP | P49908 (SEPP1_HUMAN) |
| 276. | SERPINA4 | P29622 (KAIN_HUMAN) |
| 277. | KNG1 | P01042 (KNG1_HUMAN) |
| 278. | C9 | P02748 (CO9_HUMAN) |
| 279. | PROS1 | P07225 (PROS_HUMAN) |
| 280. | CFHR2 | P36980 (FHR2_HUMAN) |
| 281. | SPON1 | Q9HCB6 (SPON1_HUMAN) |
| 282. | CLSTN1 | O94985 (CSTN1_HUMAN) |
| 283. | APP | P05067 (A4_HUMAN) |
| 284. | GPX3 | P22352 (GPX3_HUMAN) |
| 285. | RNASE1 | P07998 (RNAS1_HUMAN) |
| 286. | IGFBP6 | P24592 (IBP6_HUMAN) |
| 287. | AL645922.1 | B4E1Z4 (B4E1Z4_HUMAN) NM_001710 |
| 288. | ITIH2 | P19823 (ITIH2_HUMAN) |
| 289. | C5 | P01031 (CO5_HUMAN) |
| 290. | GSN | P06396 (GELS_HUMAN) |
| 291. | APOA2 | P02652 (APOA2_HUMAN) |
| 292. | RBP3 | P10745 (RET3_HUMAN) |
| 293. | HP | P00738 (HPT_HUMAN) |
| 294. | C3 | P01024 (CO3_HUMAN) |
| 295. | APOA4 | P06727 (APOA4_HUMAN) |
| 296. | C6 | P13671 (CO6_HUMAN) |
| 297. | APLP2 | Q06481 (APLP2_HUMAN) |
| 298. | TTR | P02766 (TTHY_HUMAN) |
| 299. | PLG | P00747 (PLMN_HUMAN) |

Embodiments of this invention further contemplate processes for determining a level of a biomarker of the separated, isolated or enriched extracellular matrix bodies. The biomarker may be the level of the extracellular matrix bodies, or the level of a substance found in the extracellular matrix bodies. Examples of substances include proteins, polypeptides, lipid molecules, lipoparticles, carbohydrates, nucleic acid molecules, and expression levels of one or more nucleic acids.

In certain embodiments, the level of extracellular matrix bodies may be determined by microscopy.

As used herein, a level of extracellular matrix bodies can be an amount of extracellular matrix bodies. For example, an amount of extracellular matrix bodies can be proportional to the signal of a marker within the extracellular matrix bodies, or proportional to an image area of a stained moiety within the extracellular matrix bodies, or proportional to an analytical spectral signal of the extracellular matrix bodies, or a mass of the extracellular matrix bodies.

In additional aspects, the level of a substance may be determined by any analyte technique including immunostaining, fluorescence assay, chelate complexation, quantitative HPLC, spectrophotometry, antibody array, Western blot, immunoassay, immunoprecipitation, ELISA, LC-MS, LC-MRM, radioimmunoassay, mass spectrometry, 2D gel mass spectrometry, LC-MS/MS, RT-PCR, and combinations thereof.

In further aspects, the level of certain substances, or their nature and/or composition may be determined by nucleic acid assay or sequencing, or next generation sequencing.

In further aspects, the level of a substance may be determined by imaging techniques include electron microscopy, stereoscopic microscopy, wide-field microscopy, polarizing microscopy, phase contrast microscopy, multiphoton microscopy, differential interference contrast microscopy, fluorescence microscopy, laser scanning confocal microscopy, multiphoton excitation microscopy, ray microscopy, and ultrasonic microscopy.

In some embodiments, the level of a substance may be determined by imaging techniques including positron emission tomography, computerized tomography, and magnetic resonance imaging.

In some embodiments, the level of a substance may be determined by assay techniques including colorimetric assay, chemiluminescence assay, spectrophotometry, immunofluorescence assay, and light scattering.

Examples of methods for analyzing extracellular matrix bodies include microscopy, mass spectrometry, microarray, nucleic acid amplification, hybridization, fluorescence hybridization, immunohistochemistry, nucleic acid analysis or sequencing, next generation sequencing, flow cytometry, chromatography, electrophoresis, and combinations thereof.

### Extracellular Matrix Bodies and Diagnosis of Disease

Embodiments of this invention can provide processes for diagnosing, prognosing or monitoring a disease in a subject. Biomarker levels obtained by separating, isolating, or enriching extracellular matrix bodies can be used for medical or diagnostic uses.

In some aspects, biomarker levels may be obtained from extracellular matrix bodies by centrifugation and/or filtration, and in various combinations, as described herein. Subsequently, a level of one or more biomarkers based on the extracellular matrix bodies that were separated, isolated or enriched can be determined.

In certain embodiments, a biomarker level can be the quantity of extracellular matrix bodies themselves.

In further embodiments, a biomarker level can be the quantity of a substance found in the extracellular matrix bodies, such as a protein, a polypeptide, a lipid molecule, a lipoparticle, a carbohydrate, a nucleic acid molecule, or an expression level of a nucleic acid.

Processes for diagnosing, prognosing or monitoring a disease in a test subject may compare the level of one or more biomarkers from a sample of the test subject to a reference level based on a control group of subjects. In some embodiments, the comparison may result in a diagnosis, prognosis or monitor the state or progression of the disease in the subject.

In certain embodiments, the comparing the level of one or more biomarkers from a sample of the test subject to a reference level based on a control group of subjects can include determining differences between a level of a biomarker and a reference level. A difference between a level of a biomarker and a reference level may also be a deviation of a level of a biomarker from a reference level.

In certain embodiments, the comparing the level of the biomarkers to a reference level based on a control group of subjects can include determining differences between a level of a biomarker and a reference level. A difference between a level of a biomarker and a reference level may also be a deviation of a level of a biomarker from a reference level.

In certain embodiments, the comparing the levels of the biomarkers to reference levels based on a control group of subjects can include determining differences between a level of a biomarker and a reference level. A difference between a level of a biomarker and a reference level may also be a deviation of a level of a biomarker from a reference level.

In some embodiments, a control group may be composed of subjects having the same disease as the test subject. In certain embodiments, a control group may be composed of subjects not clinically known to have a disease similar to the test subject. In further embodiments, a control group may be composed of healthy subjects.

In further embodiments, biomarker levels determined from separated, isolated or enriched extracellular matrix bodies can be combined with any number of known biomarkers of a particular disease to improve processes for diagnosing, prognosing or monitoring the disease.

In some embodiments, this invention can provide methods for early detection of disease in a subject. The methods include obtaining a biological sample from the subject, isolating extracellular matrix bodies from the sample, and determining the presence of the disease in the subject from a level of the isolated extracellular matrix bodies or a level of a biomarker contained in the extracellular matrix bodies. The presence of the disease in the subject may be determined before any one of:
onset of clinical signs and symptoms of the disease in the subject,
treatment for the disease is recommended or administered based on clinical examination of the subject, and
disease is detected in the subject by needle or tissue biopsy.

In some embodiments, treating a subject for a disease may be by any one or more of surgery, drug therapy, therapeutic radiation, and chemotherapy.

### Preparing Samples for Medical Information

Aspects of this invention include isolating and preserving the composition and properties of extracellular matrix bodies from a biological fluid or material. By preserving the composition and properties of extracellular matrix bodies isolated or extracted from a biological sample, fluid or material, the extracellular matrix bodies can be used, for example, for diagnosis or medical information, assaying activity of an agent against a disease or condition, or for monitoring biochemical or biological processes or changes of the sample material. Embodiments of this invention can be used to isolate, extract, and utilize extracellular matrix bodies that are a source of multiple and specific biomarkers.

A sample fluid of this disclosure may contain a carrier fluid, a biofluid, and/or reagents of interest. Examples of a carrier include water, purified water, saline solution, buffers, and organic solvents. A sample fluid may contain a gelling agent, a surfactant, or reagents for interacting with biological components.

Additional methods of this disclosure include preparing a biological sample for a diagnostic, prognostic, clinical or therapeutic use by isolating extracellular matrix bodies from the biological sample. The biological sample may be composed of bodily fluid, homogenized tissue, lysed cells, and/or lysed vesicles.

Examples of biological fluid include any bodily fluid, whole blood, blood plasma, blood serum, cerebrospinal fluid, vitreous humor, aqueous humor, urine, saliva, sweat, tears, synovial fluid, pleural fluid, gastric fluid, peritoneal fluid, breast milk, nipple aspirate, ocular fluid, semen, amniotic fluid, lymph, bile, cerumen, chyle, chyme, endolymph, perilymph, exudates, feces, ejaculate, gastric acid, gastric juice, mucus, pericardial fluid, pus, rheum, sebum, serous fluid, smegma, sputum, synovial fluid, vaginal secretion, menstrual effluent, vomit and combinations thereof.

The biological fluid may preferably be any of whole blood, blood plasma, blood serum, cerebrospinal fluid, vitreous humor, aqueous humor, urine, saliva, and combinations thereof.

Embodiments of this invention include methods for preparing a biological sample for a medical, diagnostic or prognostic use by isolating extracellular matrix bodies from the biological sample. Extracellular matrix bodies of biological sample such as a bodily fluid may be isolated by centrifugation, filtration, or a combination thereof as described herein.

A kit of this invention for a medical, diagnostic or prognostic use of extracellular matrix bodies may contain one or more reagents for measuring a biomarker level or quantity as disclosed herein, and comparing the biomarker level to a control. A kit of this invention may contain one or more reagents for measuring one or more proteins disclosed in Table 1 herein.

In certain embodiments, the comparing the level of one or more biomarkers from a sample of the test subject to a reference level based on a control group of subjects can include determining differences between a level of a biomarker and a reference level. A difference between a level of a biomarker and a reference level may also be a deviation of a level of a biomarker from a reference level.

In certain embodiments, the comparing the level of the biomarkers to a reference level based on a control group of subjects can include determining differences between a level of a biomarker and a reference level. A difference between a level of a biomarker and a reference level may also be a deviation of a level of a biomarker from a reference level.

### Extracted Compositions and Methods of Use

A composition of this invention may be composed of separated, isolated or enriched extracellular matrix bodies, which may be used in treatment of the human or animal body. The extracellular matrix bodies may be associated with pathology of a disease.

A composition of extracellular matrix bodies, isolated and/or extracted, can be combined with a pharmaceutical carrier and one or more pharmaceutical excipients.

A composition of this invention may be composed of a fraction of a bodily fluid in which extracellular matrix bodies have been separated, isolated or enriched. The composition may be used in treatment of the human or animal body. The extracellular matrix bodies may be associated with pathology of a disease.

In further embodiments, a composition may comprise a sample from which extracellular matrix bodies have been removed by the isolation and/or extraction processes for use in the treatment of the human or animal body. In certain embodiments, at least 25%, or at least 50%, or at least 75%, or at least 90%, or substantially all of the extracellular matrix bodies of a sample have been removed by the isolation and/or extraction processes herein for use in the treatment of the human or animal body.

### Fixation of Extracellular Matrix Bodies

Embodiments of this invention further include detecting extracellular matrix bodies in a biological fluid by fixation. These methods include contacting the biological fluid with a non-reversible cross-linking agent which fixes the extracellular matrix bodies.

Embodiments of this invention further include fixation of extracellular matrix bodies on a glass surface using a carbodiimide agent. In certain embodiments, a carbodiimide agent can be 1-Ethyl-3-(3-dimethylaminopropyl) carbodiimide.

This disclosure provides methods for detecting extracellular matrix bodies in a biological fluid by fixation. Methods involve contacting the biological fluid with a non-reversible cross-linking agent which fixes the extracellular matrix bodies. In some embodiments, a non-reversible cross-linking agent can be a water-soluble carbodiimide, a cyanogen halide, or a mixture thereof. In certain embodiments, a non-reversible cross-linking agent may be 1-ethyl-3-(3-dimethylaminopropyl)-carbodiimide, cyanogen bromide, cyanogen fluoride, cyanogen chloride, cyanogen iodide, or combinations thereof. Subsequently, the biological fluid may be contacted with an aldehyde-containing fixative agent.

Extracellular matrix bodies that have been fixed may be detected by any form of microscopy, spectrophotometry, tomography, or magnetic resonance.

In certain embodiments, a biological fluid may be any one of whole blood, blood plasma, blood serum, cerebrospinal fluid, vitreous humor, aqueous humor, urine, saliva, sweat, tears, synovial fluid, pleural fluid, gastric fluid, peritoneal fluid, breast milk, nipple aspirate, ocular fluid, semen, amniotic fluid, lymph, bile, cerumen, chyle, chyme, endolymph, perilymph, exudates, feces, ejaculate, gastric acid, gastric juice, mucus, pericardial fluid, pus, rheum, sebum, serous fluid, smegma, sputum, synovial fluid, vaginal secretion, menstrual effluent, vomit and combinations thereof.

In further aspects, this invention can provide kits for fixing extracellular matrix bodies in a biological fluid. A kit may contain a support substrate for holding the biological fluid and a non-reversible cross-linking agent. A kit may further comprise an aldehyde-containing fixative agent.

Numbered embodiments of this invention may include:

1) A process for separating, isolating or enriching extracellular matrix bodies in a biological fluid, the process comprising centrifuging or filtering the biological fluid.

2) The process of embodiment 1, comprising
removing cells or cell debris by centrifuging the biological fluid; and
separating, isolating or enriching extracellular matrix bodies from the biological fluid by centrifuging or filtering the biological fluid.

3) The process of embodiment 2, wherein the removing cells or cell debris by centrifuging the biological fluid comprises low speed centrifugation.

4) The process of embodiment 2, wherein the removing cells or cell debris by centrifuging the biological fluid comprises centrifugation which applies about 200 to 5,000 g for 1-10 minutes, or 100 to 1,200 g for 1-10 minutes.

5) The process of any of embodiments 1-4, wherein the separating, isolating or enriching extracellular matrix bodies from the biological fluid by centrifuging creates a pellet by applying forces for times and speeds below levels needed to force small particles into the pellet, so that the pellet is substantially free of particles smaller than about 1 micrometer.

6) The process of any of embodiments 1-5, wherein the separating, isolating or enriching extracellular matrix bodies from the biological fluid by centrifuging creates a pellet by applying 3,000-20,000 g for 1-100 minutes, or 6,000-100,000 g for 1-200 minutes.

7) The process of any of embodiments 1-6, wherein the separating, isolating or enriching extracellular matrix bodies from the biological fluid by centrifuging creates a pellet by applying 3,000-20,000 g for 1-100 minutes, or 6,000-100,000 g for 1-200 minutes, and re-suspending the pellet.

8) The process of any of embodiments 1-7, wherein the separating, isolating or enriching extracellular matrix bodies from the biological fluid by filtering is performed with a filter for passing particles of less than a cutoff size, wherein the cutoff size is 1, or 2, or 3, or 4, or 5, or 6, or 10 micrometers, and the extracellular matrix bodies are extracted from the filter and re-suspended.

9) The process of any of embodiments 1-8, comprising additional separating, isolating or enriching extracellular matrix bodies from a supernatant after centrifuging the biological fluid, or from a retentate, precipitate, residue, or extract after filtering the biological fluid, or from a filtrate after filtering the biological fluid, wherein the additional separating, isolating or enriching is done by microfluidic device, centrifugation, chromatography, chemical precipitation, filtration, immuno- or affinity-capture, electrophoresis, AC electrokinetics, or a combination thereof.

10) The process of any of embodiments 1-9, wherein the separating, isolating or enriching captures at least a majority of the extracellular matrix bodies from the biological fluid.

11) The process of any of embodiments 1-10, wherein the separating, isolating or enriching captures at least a majority of the extracellular matrix bodies from the biological fluid with an absence of cells.

12) The process of any of embodiments 1-11, wherein the separating, isolating or enriching captures substantially all of the extracellular matrix bodies from the biological fluid.

13) The process of any of embodiments 1-12, wherein the separating, isolating or enriching captures substantially all of the extracellular matrix bodies from the biological fluid with an absence of cells.

14) The process of any of embodiments 1-13, wherein the level of separated, isolated or enriched extracellular matrix bodies is a biomarker for medical, diagnostic or prognostic information.

15) The process of any of embodiments 1-14, wherein the separated, isolated or enriched extracellular matrix bodies comprise biomarkers in the form of a protein, a polypeptide, a lipid molecule, a lipoparticle, a carbohydrate, a nucleic acid molecule, or an expression level of a nucleic acid.

16) The process of any of embodiments 1-15, wherein the separated, isolated or enriched extracellular matrix bodies comprise biomarkers in the form of extracellular proteins, RNA, DNA, or a protein in Table 1.

17) A process for separating, isolating or enriching extracellular matrix bodies in a biological fluid, the process comprising
applying an initial centrifugation step to the biological fluid for removing cells or cell debris; and
applying one or more serial centrifugation steps to the supernatant of the initial centrifugation, wherein a supernatant is used in each step.

18) The process of embodiment 17, wherein the removing cells or cell debris by centrifuging the biological fluid comprises low speed centrifugation.

19) The process of embodiment 17 or embodiment 18, wherein the removing cells or cell debris by centrifuging the biological fluid comprises centrifugation which applies about 200 to 5,000 g for 1-10 minutes, or 100 to 1,200 g for 1-10 minutes.

20) The process of any of embodiments 17-19, wherein the one or more serial centrifugation steps each apply about 1,000 to 60,000 g for 1-100 minutes, or 1,000 to 3,000 g for 1-30 minutes to the supernatant from the previous step.

21) The process of any of embodiments 17-20, comprising filtering the supernatant from a step of serial centrifugation with a filter for passing particles of less than a cutoff size; wherein the cutoff size is 1, or 2, or 3, or 4, or 5, or 6, or 10 micrometers, and extracting extracellular matrix bodies trapped in the filter.

22) The process of any of embodiments 17-21, comprising additional separating, isolating or enriching extracellular matrix bodies from a supernatant after centrifuging the biological fluid, wherein the additional separating, isolating or enriching is done by microfluidic device, centrifugation, chromatography, chemical precipitation, filtration, immuno- or affinity-capture, electrophoresis, AC electrokinetics, or a combination thereof.

23) The process of any of embodiments 17-22, wherein the separating, isolating or enriching captures at least a majority of the extracellular matrix bodies from the biological fluid.

24) The process of any of embodiments 17-23, wherein the separating, isolating or enriching captures at least a majority of the extracellular matrix bodies from the biological fluid with an absence of cells.

25) The process of any of embodiments 17-24, wherein the separating, isolating or enriching captures substantially all of the extracellular matrix bodies from the biological fluid.

26) The process of any of embodiments 17-25, wherein the separating, isolating or enriching captures substantially all of the extracellular matrix bodies from the biological fluid with an absence of cells.

27) The process of any of embodiments 17-26, wherein the level of separated, isolated or enriched extracellular matrix bodies is a biomarker for medical, diagnostic or prognostic information.

28) The process of any of embodiments 17-27, wherein the separated, isolated or enriched extracellular matrix bodies comprise biomarkers in the form of a protein, a polypeptide, a lipid molecule, a lipoparticle, a carbohydrate, a nucleic acid molecule, or an expression level of a nucleic acid.

29) The process of any of embodiments 17-28, wherein the separated, isolated or enriched extracellular matrix bodies comprise biomarkers in the form of extracellular proteins, RNA, DNA, or a protein in Table 1.

30) The process of any of embodiments 1-29, comprising separating, isolating or enriching extracellular matrix bodies by:
re-suspending a pellet obtained from any preceding step;
filtering the re-suspension with a filter for passing particles of less than a cutoff size, wherein the cutoff size is 1, or 2, or 3, or 4, or 5, or 6, or 10 micrometers; and
extracting extracellular matrix bodies trapped in the filter.

31) The process of embodiment 30, wherein the filtering captures at least a majority of the extracellular matrix bodies from the biological fluid, and the re-suspension is substantially free of particles smaller than about 1 micrometer.

32) The process of embodiment 30 or embodiment 31, wherein the filtering captures substantially all of the extracellular matrix bodies from the biological fluid, and the re-suspension is substantially free of particles smaller than about 1 micrometer.

33) The process of any of embodiments 30-32, comprising adding a reagent to a supernatant after the initial centrifugation, wherein the reagent is for precipitating the extracellular matrix bodies.

34) The process of any of embodiments 30-33, wherein the extracellular matrix bodies are at least 2-fold, or at least 5-fold, or at least 10-fold enriched in concentration as compared to the biological fluid.

35) The process of any of embodiments 30-34, wherein the extracellular matrix bodies are associated with a pathology or disease.

36) The process of any of embodiments 1-35, wherein the biological fluid is any one of whole blood, blood plasma, blood serum, cerebrospinal fluid, vitreous humor, aqueous humor, urine, saliva, sweat, tears, synovial fluid, pleural fluid, gastric fluid, peritoneal fluid, breast milk, nipple aspirate, ocular fluid, semen, amniotic fluid, lymph, bile, cerumen, chyle, chyme, endolymph, perilymph, exudates, feces, ejaculate, gastric acid, gastric juice, mucus, pericardial fluid, pus, rheum, sebum, serous fluid, smegma, sputum, synovial fluid, vaginal secretion, menstrual effluent, vomit and combinations thereof.

37) The process of any of embodiments 1-36, comprising determining a level of a biomarker of the separated, isolated or enriched extracellular matrix bodies.

38) The process of any of embodiments 1-37, wherein the biomarker is the level of the extracellular matrix bodies, or the level of a substance found in the extracellular matrix bodies, wherein the substance is a protein, a polypeptide, a lipid molecule, a lipoparticle, a carbohydrate, a nucleic acid molecule, or an expression level of a nucleic acid.

39) The process of any of embodiments 1-38, wherein the level of the extracellular matrix bodies is determined by microscopy.

40) The process of any of embodiments 1-39, wherein the level of the substance is determined by any of immunostaining, fluorescence assay, chelate complexation, quantitative HPLC, spectrophotometry, antibody array, Western blot, immunoassay, immunoprecipitation, ELISA, LC-MS, LC-MRM, radioimmunoassay, mass spectrometry, 2D gel mass spectrometry, LC-MS/MS, RT-PCR, nucleic acid assay, next generation sequencing, and combinations thereof.

41) A process for diagnosing, prognosing or monitoring a disease in a subject, the process comprising
separating, isolating or enriching extracellular matrix bodies in a biological fluid sample of the subject;
determining a level of one or more biomarkers based on the separated, isolated or enriched extracellular matrix bodies, wherein the biomarker is the level of the extracellular matrix bodies, or the level of a substance found in the extracellular matrix bodies, wherein the substance is a protein, a polypeptide, a lipid molecule, a lipoparticle, a carbohydrate, a nucleic acid molecule, or an expression level of a nucleic acid; and
comparing the levels of the biomarkers to reference levels based on a control group of subjects, and diagnosing, prognosing or monitoring the disease in the subject.

42) The process of embodiment 41, wherein the separated, isolated or enriched extracellular matrix bodies comprise biomarkers in the form of proteins, extracellular matrix proteins, polypeptides, lipids, lipoparticles, carbohydrates, nucleic acid molecules, DNA, or an expression level of a nucleic acid.

43) The process of embodiment 41 or embodiment 42, wherein the separating, isolating or enriching extracellular matrix bodies in a biological fluid sample of the subject comprises performing a process according to any one of embodiments 1 to 40.

44) The process of any of embodiments 41-43, comprising treating the subject for the disease by any one or more of surgery, drug therapy, therapeutic radiation, and chemotherapy.

45) A composition comprising extracellular matrix bodies isolated by the process of any one of embodiments 1-44.

46) The composition of embodiments 45, wherein the extracellular matrix bodies are associated with pathology of a disease.

47) A composition of embodiment 45 or embodiment 46, for use in a method of therapy of a human or animal body.

48) A method for preparing a biological sample for a medical, diagnostic or prognostic use, the method comprising isolating extracellular matrix bodies from the biological sample according to the process of any of embodiments 1-44, wherein the extracellular matrix bodies have a principal size from about 1 micrometer to 200 micrometers, or from about 4 micrometers to 200 micrometers.

49) The method of embodiment 48, wherein the biological sample is composed of a bodily fluid.

50) The method of embodiment 49, wherein the bodily fluid is any of whole blood, blood plasma, blood serum, cerebrospinal fluid, vitreous humor, aqueous humor, urine, saliva, sweat, tears, synovial fluid, pleural fluid, gastric fluid, peritoneal fluid, breast milk, nipple aspirate, ocular fluid, semen, amniotic fluid, lymph, bile, cerumen, chyle, chyme, endolymph, perilymph, exudates, feces, ejaculate, gastric acid, gastric juice, mucus, pericardial fluid, pus, rheum, sebum, serous fluid, smegma, sputum, synovial fluid, vaginal secretion, menstrual effluent, vomit and combinations thereof.

51) A method for preparing a sample by distinguishing extracellular matrix bodies in a biological fluid or material or tissue for a medical, diagnostic or prognostic use, the method comprising:
isolating extracellular matrix bodies from the biological sample according to the process of any of embodiments 1-44, wherein the extracellular matrix bodies have a principal size from about 1 micrometer to 200 micrometers, or from about 4 micrometers to 200 micrometers.

52) The method of embodiment 51, wherein the biological sample is composed of a bodily fluid.

53) The method of embodiment 52, wherein the bodily fluid is any of whole blood, blood plasma, blood serum, cerebrospinal fluid, vitreous humor, aqueous humor, urine, saliva, sweat, tears, synovial fluid, pleural fluid, gastric fluid, peritoneal fluid, breast milk, nipple aspirate, ocular fluid, semen, amniotic fluid, lymph, bile, cerumen, chyle, chyme, endolymph, perilymph, exudates, feces, ejaculate, gastric acid, gastric juice, mucus, pericardial fluid, pus, rheum, sebum, serous fluid, smegma, sputum, synovial fluid, vaginal secretion, menstrual effluent, vomit and combinations thereof.

54) A method for preparing a sample of extracellular matrix bodies in a biological fluid by fixation, the method comprising contacting the biological fluid with a non-reversible cross-linking agent which fixes the extracellular matrix bodies.

55) The method of embodiment 54, wherein the non-reversible cross-linking agent is a water-soluble carbodiimide, a cyanogen halide, or a mixture thereof.

56) The method of embodiment 54 or embodiment 55, wherein the non-reversible cross-linking agent is 1-ethyl-3-(3-dimethylaminopropyl)-carbodiimide, cyanogen bromide, cyanogen fluoride, cyanogen chloride, or cyanogen iodide.

57) The method of any of embodiments 54-56, comprising contacting the biological fluid with an aldehyde-containing fixative agent.

58) The method of any of embodiments 54-57, comprising detecting the fixed extracellular matrix bodies by microscopy, spectrophotometry, tomography, or magnetic resonance.

59) The method of any of embodiments 54-58, wherein the biological fluid is any of whole blood, blood plasma, blood serum, cerebrospinal fluid, vitreous humor, aqueous humor, urine, saliva, sweat, tears, synovial fluid, pleural fluid, gastric fluid, peritoneal fluid, breast milk, nipple aspirate, ocular fluid, semen, amniotic fluid, lymph, bile, cerumen, chyle, chyme, endolymph, perilymph, exudates, feces, ejaculate, gastric acid, gastric juice, mucus, pericardial fluid, pus, rheum, sebum, serous fluid, smegma, sputum, synovial fluid, vaginal secretion, menstrual effluent, vomit and combinations thereof.

60) A kit for fixing extracellular matrix bodies in a biological fluid, comprising:
a support substrate for holding the biological fluid; and
a non-reversible cross-linking agent.

61) The kit of embodiment 60, wherein the non-reversible cross-linking agent is 1-ethyl-3-(3-dimethylaminopropyl)-carbodiimide, cyanogen bromide, cyanogen fluoride, cyanogen chloride, or cyanogen iodide, and comprising an aldehyde-containing fixative agent.

All publications including patents, patent application publications, and non-patent publications referred to in this description are each expressly incorporated herein by reference in their entirety for all purposes.

Although the foregoing disclosure has been described in detail by way of example for purposes of clarity of understanding, it will be apparent to the artisan that certain changes and modifications are comprehended by the disclosure and may be practiced without undue experimentation within the scope of the appended claims, which are presented by way of illustration not limitation. This invention includes all such additional embodiments, equivalents, and modifications. This invention includes any combinations or mixtures of the features, materials, elements, or limitations of the various illustrative components, examples, and claimed embodiments.

The terms “a,” “an,” “the,” and similar terms describing the invention, and in the claims, are to be construed to include both the singular and the plural.

### EXAMPLES

Example 1. Isolating Extracellular Matrix Bodies from Bodily Fluid by Differential centrifugation. Extracellular matrix bodies were isolated from bovine vitreous humor, a bodily fluid. A frozen bovine vitreous humor specimen was thawed and homogenized. The homogenate was spun using a benchtop centrifuge (Eppendorf 5417R, F45-30-11 Eppendorf rotor). 700-800 μl of the homogenate was placed in 1 mL centrifuge tubes and spun at each of 500, 1,000, 2,000, 3,000, 4,000, 5,000, 10,000 and 12,000 g for 10 minutes at 4° C. (See also Examples 5-12). For each speed, the supernatant was collected by aspiration, placed into a 1 mL centrifuge tube and kept at 4° C. until analysis. The pellet was collected by resuspension in 400 μL of phosphate buffered saline (PBS) and placed into a 1 mL centrifuge tube and kept at 4° C. until analysis.

To detect extracellular matrix bodies in the samples, supernatant and pellet resuspension from each centrifugation speed were immediately processed by EDC-glutaraldehyde fixation on glass slides, as described below. Samples were stained with alcian blue which is sensitive to extracellular matrix glycans. Samples were also stained with H&E stain which is sensitive to extracellular matrix cytoplasm and proteins, as well as nuclear components. Slides were preserved by mounting and sealing glass coverslips and analyzed by light microscopy.

Color bright field images were captured on a Zeiss inverted phase contrast Axiovert 200 microscope equipped with an Axiocam 105 color camera. Images were processed with Zen software (Zeiss, version 4.3).

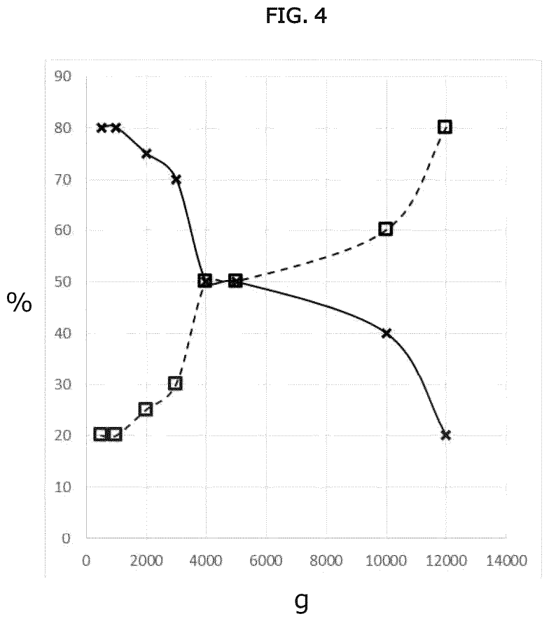

FIG. 4 shows results for differential centrifugation of a sample of a bodily fluid which was separated, isolated and enriched in extracellular matrix bodies to a high degree. FIG. 4 shows a quantitative example of separating and enriching extracellular matrix bodies from bovine vitreous humor by differential centrifugation. The relative quantities of extracellular matrix bodies isolated in a pellet (dashed line, open squares) and remaining in a supernatant (solid line, crosses) are shown. Steps of centrifugation were performed for 10 minutes each.

### Example 2. Extracellular Matrix Bodies in Healthy Control Bodily Fluids

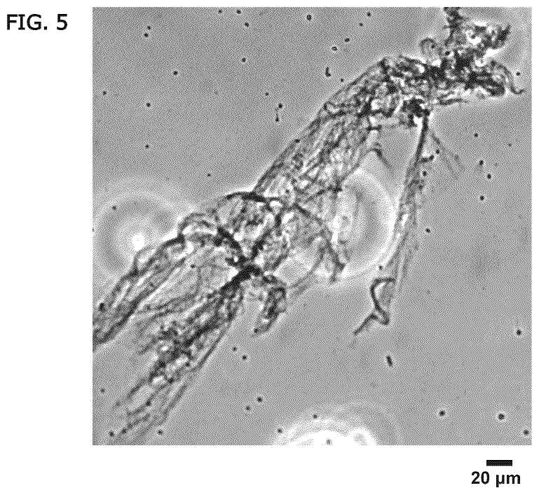

FIG. 5 shows a representative micrograph of an extracellular matrix body isolated in native human plasma obtained from a healthy control subject by light microscopy fixed on a poly-1-lysine coated glass slide and stained with hematoxylin and eosin (Vector Labs H-3502). This representative image shows an extracellular matrix body having a large structure with connected fibrous structures. The staining of the large fiber and bundle-like structures in this control indicates the presence of collagen protein. This data demonstrates visualizing the spatial localization and morphological features of a previously unknown biological material in human plasma. This image shows a distinct morphology of an extracellular matrix body present in a healthy plasma control sample.

Methods of this disclosure can be used for separating, isolating, or enriching a stable fraction of the available extracellular matrix bodies to surprisingly preserve extracellular matrix bodies having such diffuse morphology.

### Example 3. Observation of Extracellular Matrix Bodies

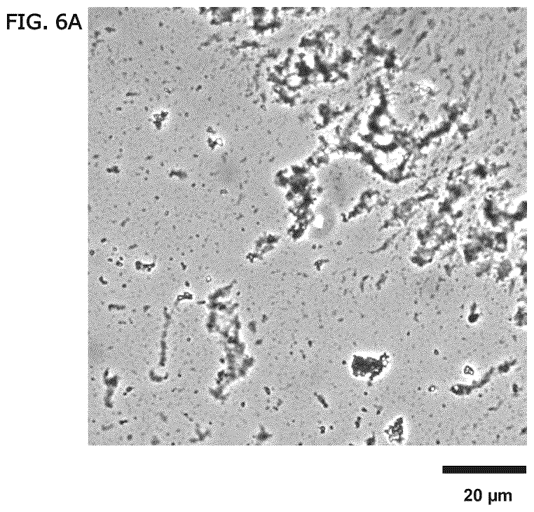

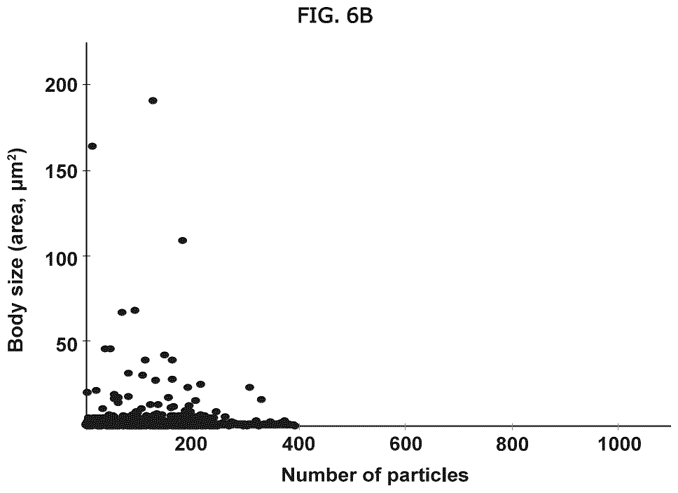

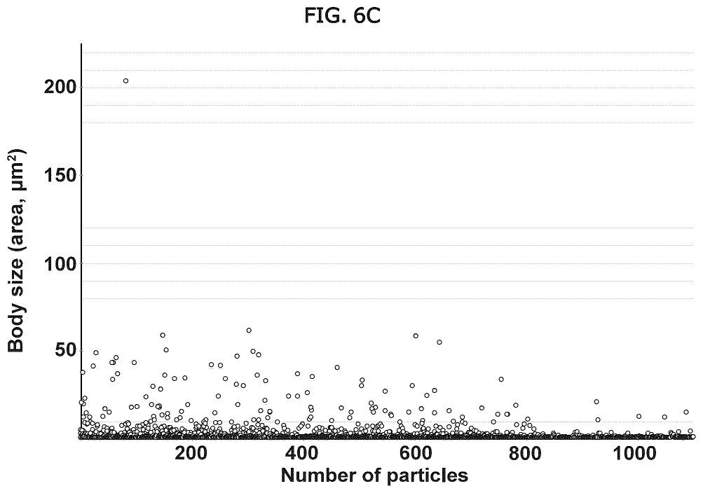

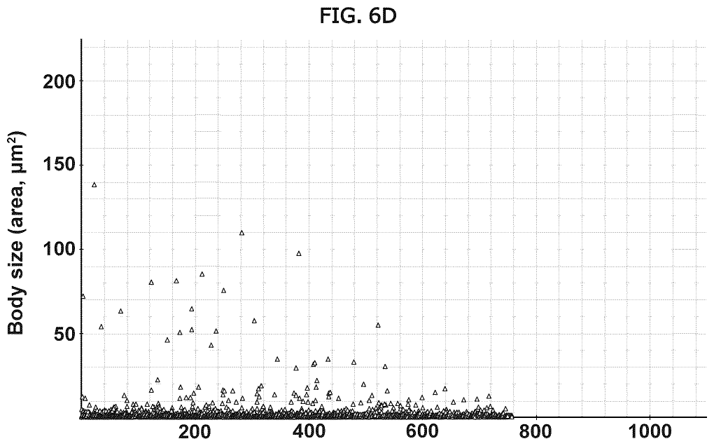

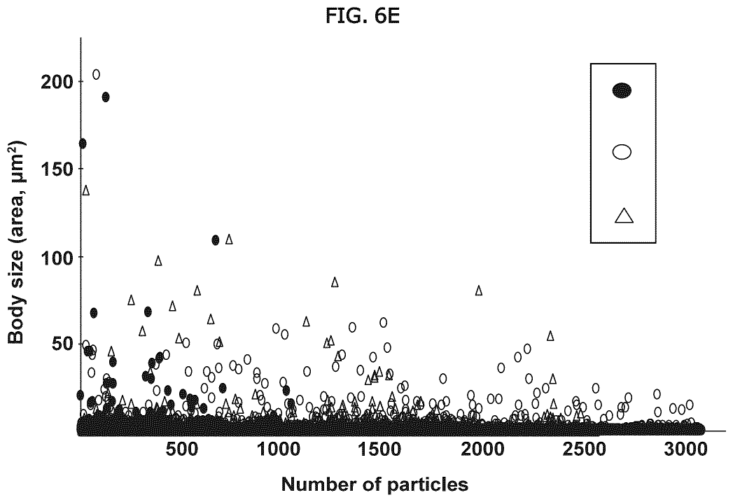

FIG. 6A shows a representative micrograph of extracellular matrix bodies isolated in native human plasma obtained from a subject having an internal disease. Image was obtained by light microscopy for extracellular matrix bodies fixed using EDC on a glass slide and stained with hematoxylin and eosin.

FIG. 6B shows a chart of the quantity of particles and their sizes for extracellular matrix bodies counted in native human plasma obtained from a non-disease subject by light microscopy fixed using EDC on a glass slide and stained with hematoxylin and eosin.

FIG. 6C shows a chart of the quantity of particles and their sizes for extracellular matrix bodies counted in native human plasma obtained from an internal disease subject by light microscopy fixed using EDC on a glass slide and stained with hematoxylin and eosin. The subject was newly diagnosed as having the internal disease.

FIG. 6D shows a chart of the quantity of particles and their sizes for extracellular matrix bodies counted in native human plasma obtained from a subject having the same internal disease as FIG. 6C, but having been diagnosed at a comparatively earlier date, and therefore having the internal disease for a longer period. Image was obtained by light microscopy fixed using EDC on a glass slide and stained with hematoxylin and eosin. Extracellular matrix bodies were more abundant and larger in size for the subject having had the internal disease for a longer period, thereby showing the use of the data for diagnosis, prognosis, and monitoring of disease.

FIG. 6E shows a combined chart of FIG. 6B, FIG. 6C and FIG. 6D.

### Example 4. Observation of Extracellular Matrix Bodies in Native Bodily Fluid by Fixation

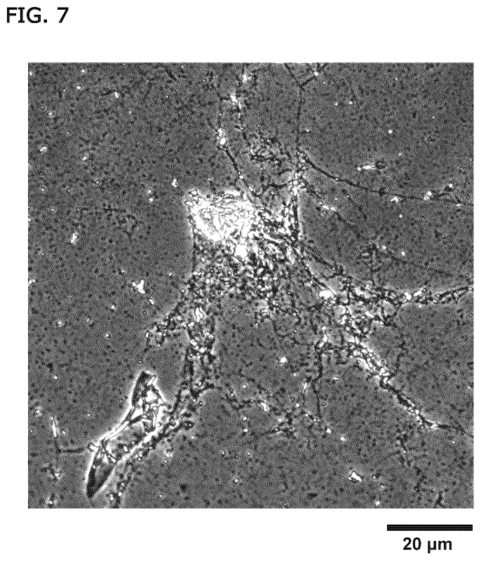

Bovine vitreous humor (BVH) is highly hydrated with 98-99.7% water content. To observe extracellular matrix bodies in native BVH, a sample of BVH was fixed by EDC-glutaraldehyde fixation on a glass slide and stained with H&E stain. FIG. 7 shows that native extracellular matrix bodies were present in the native BVH, prior to centrifugation or filtration. It was not possible to detect these extracellular matrix bodies in sufficient yield without EDC fixation, as described below in Examples 16-17.

FIG. 7 shows that the extracellular matrix bodies were present in native Bovine vitreous humor. Extracellular matrix bodies are present along with other biological material in body fluids and are observed in fluids without performing centrifugation or filtration. FIG. 7 also shows that the extracellular matrix bodies were not an artifact of applying of centrifugation or filtration to the sample vitreous humor because centrifugation and filtration were not applied to this sample.

Example 5. Isolating extracellular matrix bodies from bodily fluid by differential centrifugation. A BVH sample was placed into a centrifugation tube and spun at 500 g for 10 minutes at 4° C. Supernatant was collected and the pellet resuspended in 200 μl of PBS. Samples of the supernatant and pellet resuspension were fixed by EDC-glutaraldehyde fixation on glass slides for microscopy. Image analysis showed that 80% of the extracellular matrix bodies were found in the supernatant and 20% were found in the pellet.

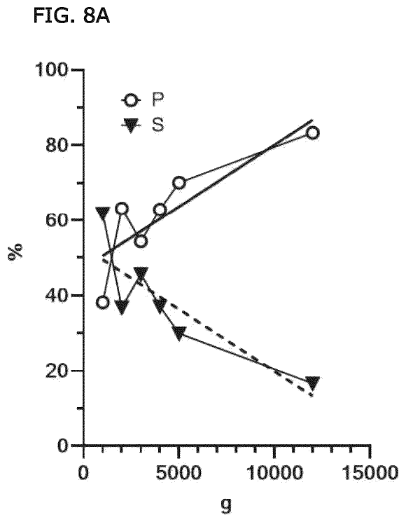

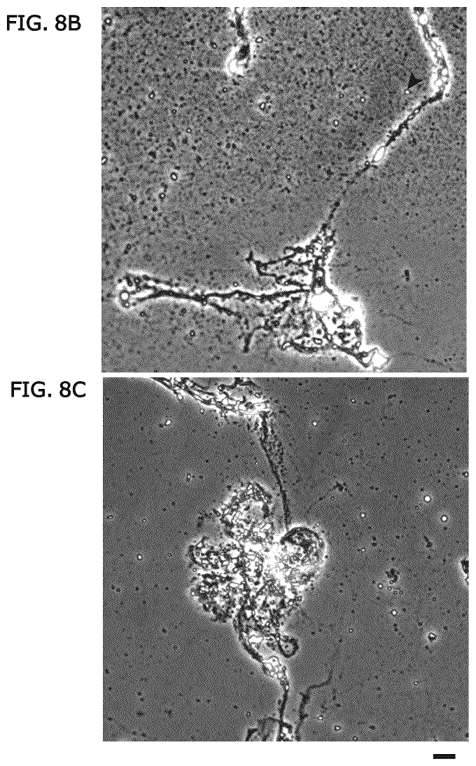

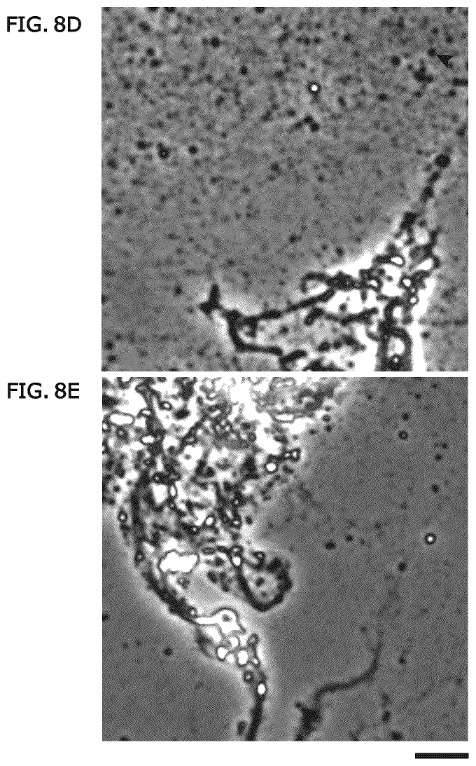

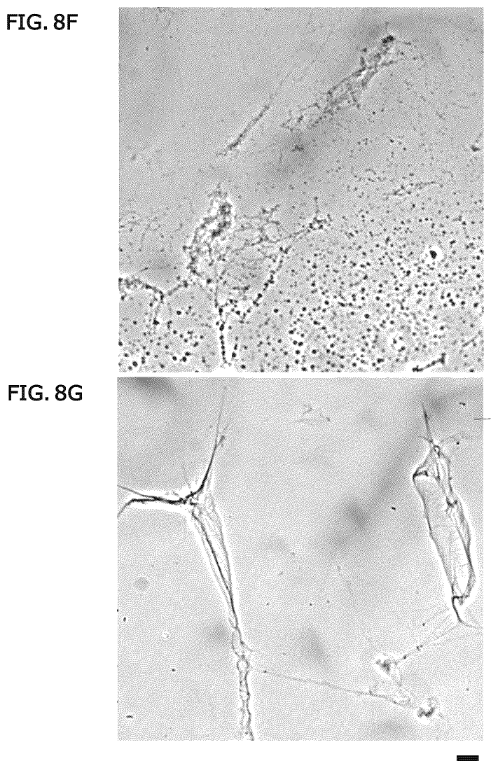

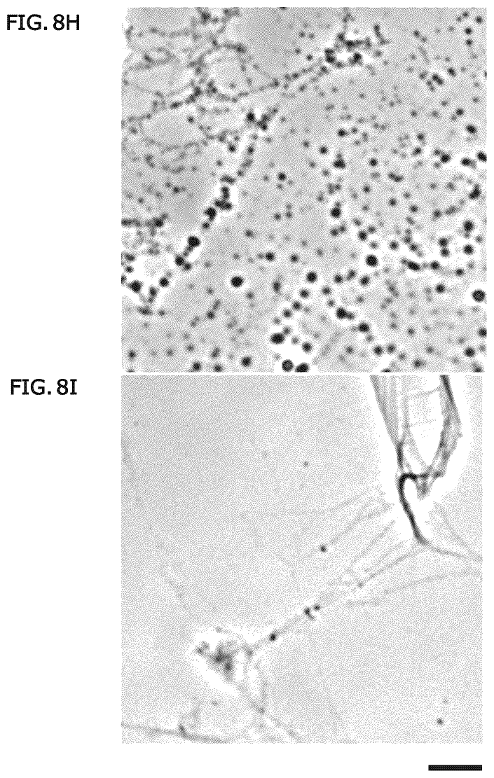

FIG. 8A shows a graph of isolation of extracellular matrix bodies in bovine vitreous humor by differential centrifugation. This graph shows the level of extracellular matrix bodies in the supernatant (S, filled triangles, dashed regression line) and pellet (P, open circles, solid regression line) after centrifugation at 1,000 to 12,000 g as a percent of total. Extracellular matrix bodies are separated to a high degree by differential centrifugation at from about 4,000 or 6,000 to about 12,000 g and higher.

FIG. 8B and FIG. 8C show micrographs of a fixed supernatant sample with H&E stain. FIG. 8E and FIG. 8E show micrographs of a fixed pellet resuspension sample with H&E stain.

FIG. 8F and FIG. 8G show micrographs of a fixed supernatant sample with alcian blue stain. FIG. 8H and FIG. 8I show micrographs of a fixed pellet resuspension sample with alcian blue stain.

Example 6. Detecting extracellular matrix bodies isolated from bodily fluid by differential centrifugation at 1,000 g. A BVH sample was placed into a centrifugation tube and spun at 1,000 g for 10 minutes at 4° C. Supernatant was collected and the pellet resuspended in 200 μl of PBS. Samples of the supernatant and pellet resuspension were fixed by EDC-glutaraldehyde fixation on glass slides for microscopy. Image analysis showed that 80% of the extracellular matrix bodies were found in the supernatant and 20% were found in the pellet.

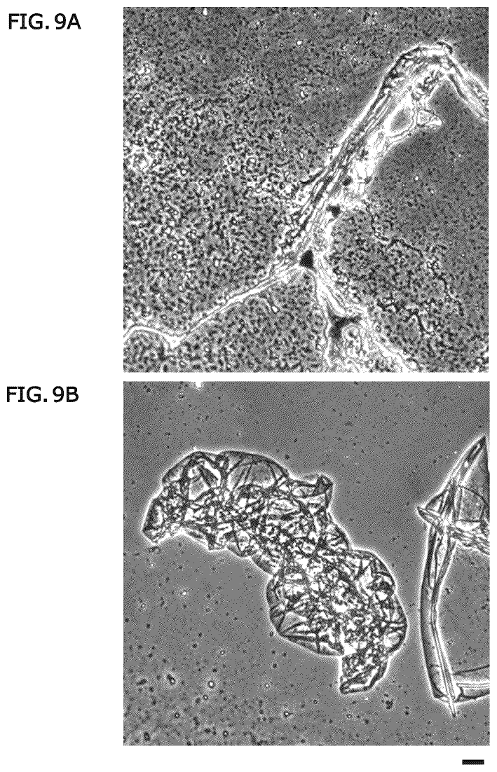

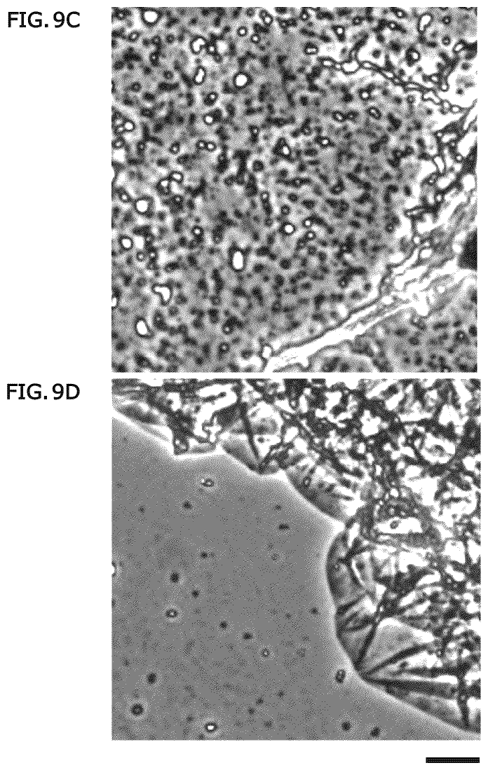

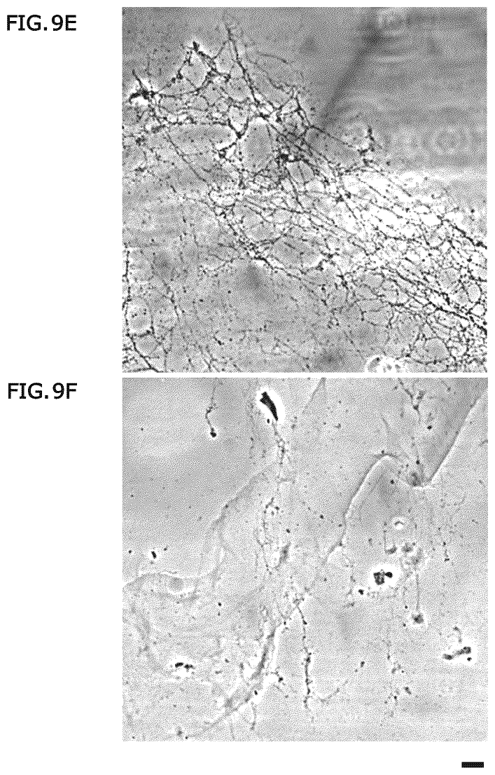

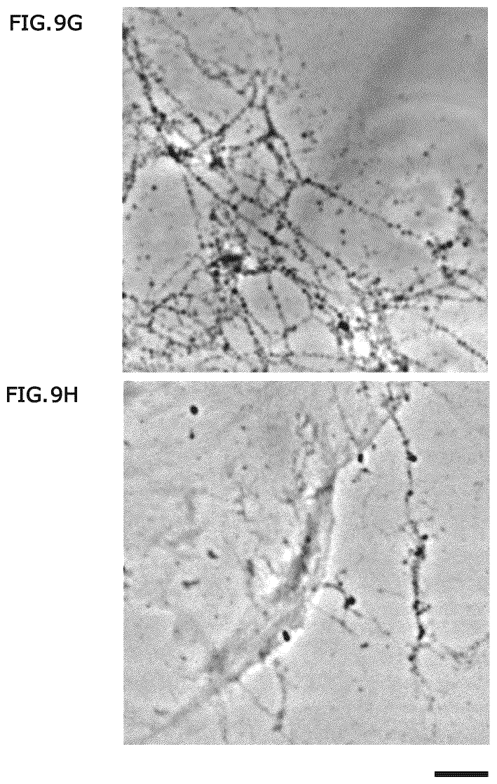

FIG. 9A and FIG. 9C show micrographs of a fixed supernatant sample with H&E stain. FIG. 9B and FIG. 9D show micrographs of a fixed pellet resuspension sample with H&E stain.

FIG. 9E and FIG. 9G show micrographs of a fixed supernatant sample with alcian blue stain. FIG. 9F and FIG. 9H show micrographs of a fixed pellet resuspension sample with alcian blue stain.

Example 7. Detecting extracellular matrix bodies isolated from bodily fluid by differential centrifugation at 2,000 g. A BVH sample was placed into a centrifugation tube and spun at 2,000 g for 10 minutes at 4° C. Supernatant was collected and the pellet resuspended in 200 μl of PBS. Samples of the supernatant and pellet resuspension were fixed by EDC-glutaraldehyde fixation on glass slides for microscopy. Image analysis showed that 75% of the extracellular matrix bodies were found in the supernatant and 25% were found in the pellet.

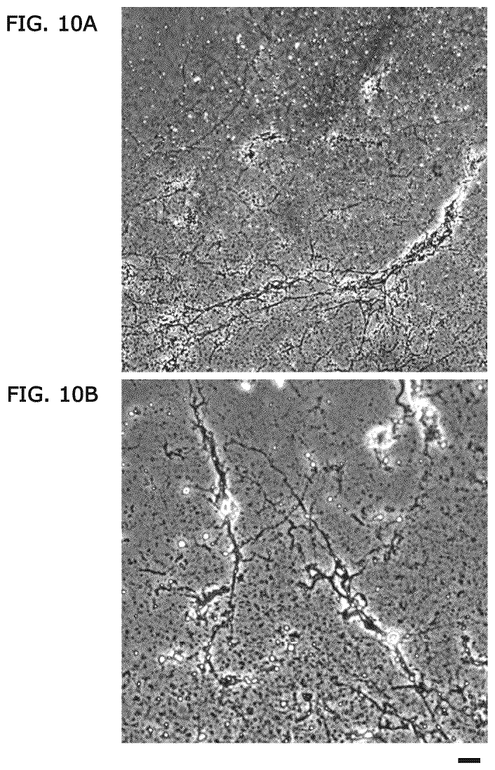

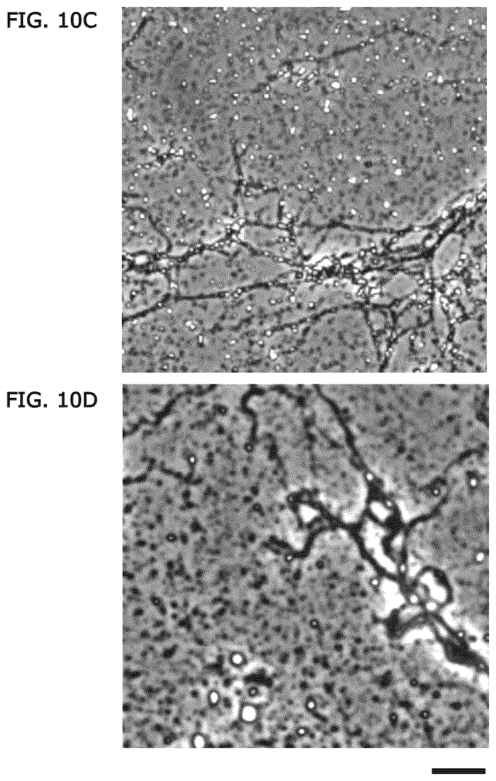

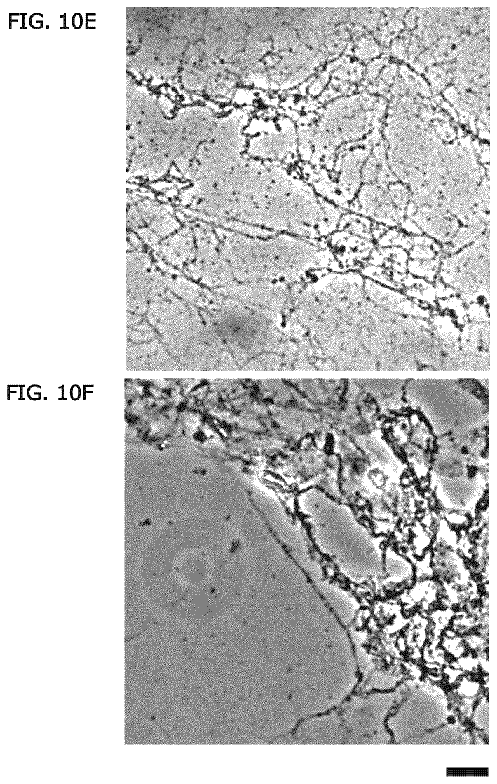

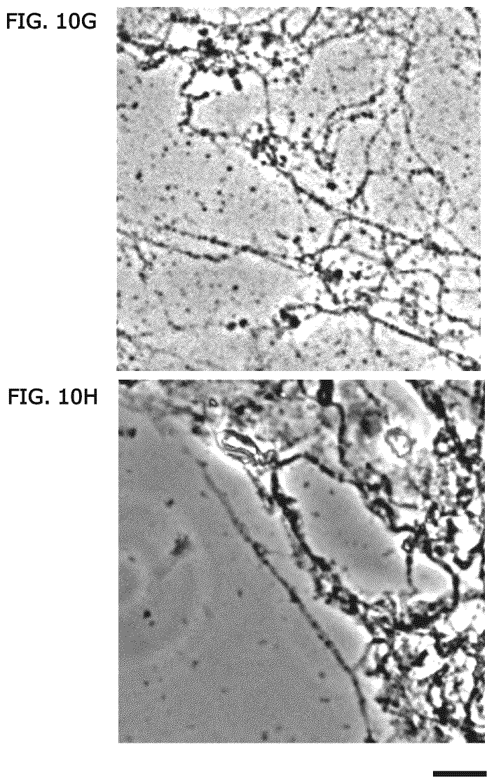

FIG. 10A and FIG. 10C show micrographs of a fixed supernatant sample with H&E stain. FIG. 10B and FIG. 10D show micrographs of a fixed pellet resuspension sample with H&E stain.

FIG. 10E and FIG. 10G show micrographs of a fixed supernatant sample with alcian blue stain. FIG. 10F and FIG. 10H show micrographs of a fixed pellet resuspension sample with alcian blue stain.

Example 8. Detecting extracellular matrix bodies isolated from bodily fluid by differential centrifugation at 3,000 g. A BVH sample was placed into a centrifugation tube and spun at 3,000 g for 10 minutes at 4° C. Supernatant was collected and the pellet resuspended in 200 μl of PBS. Samples of the supernatant and pellet resuspension were fixed by EDC-glutaraldehyde fixation on glass slides for microscopy. Image analysis showed that 70% of the extracellular matrix bodies were found in the supernatant and 30% were found in the pellet.

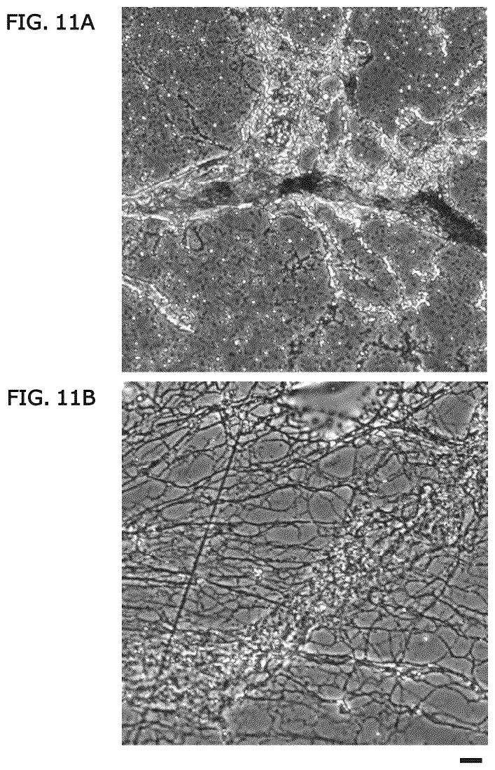

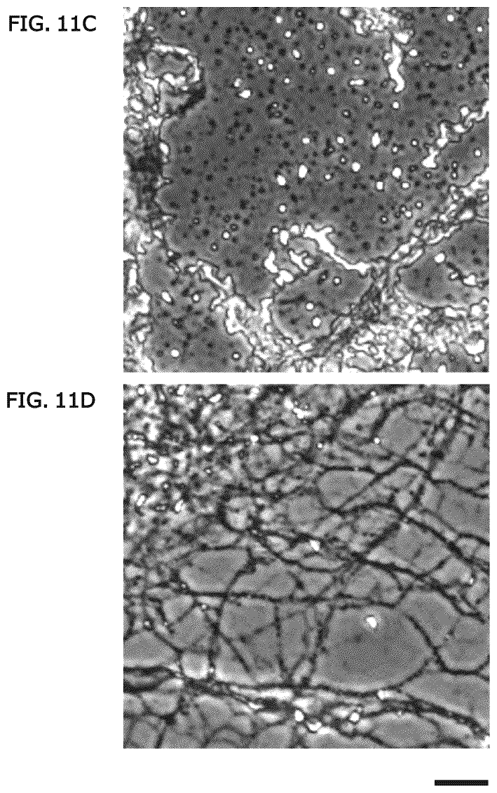

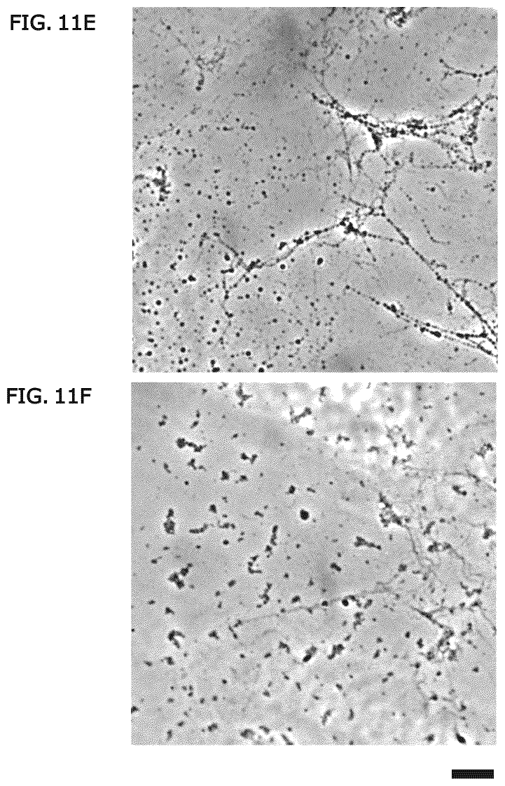

FIG. 11A and FIG. 11C show micrographs of a fixed supernatant sample with H&E stain. FIG. 11B and FIG. 11D show micrographs of a fixed pellet resuspension sample with H&E stain.

FIG. 11E shows a micrograph of a fixed supernatant sample with alcian blue stain. FIG. 11F shows a micrograph of a fixed pellet resuspension sample with alcian blue stain.

Example 9. Detecting extracellular matrix bodies isolated from bodily fluid by differential centrifugation at 4,000 g. A BVH sample was placed into a centrifugation tube and spun at 4,000 g for 10 minutes at 4° C. Supernatant was collected and the pellet resuspended in 200 μl of PBS. Samples of the supernatant and pellet resuspension were fixed by EDC-glutaraldehyde fixation on glass slides for microscopy. Image analysis showed that 50% of the extracellular matrix bodies were found in the supernatant and 50% were found in the pellet.

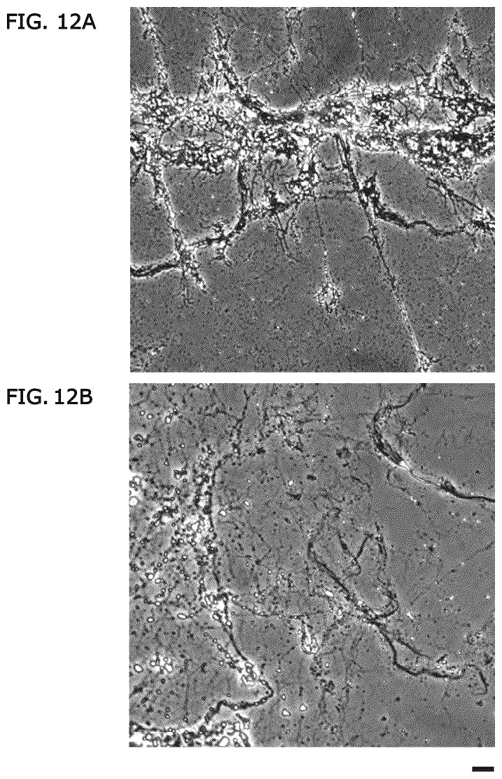

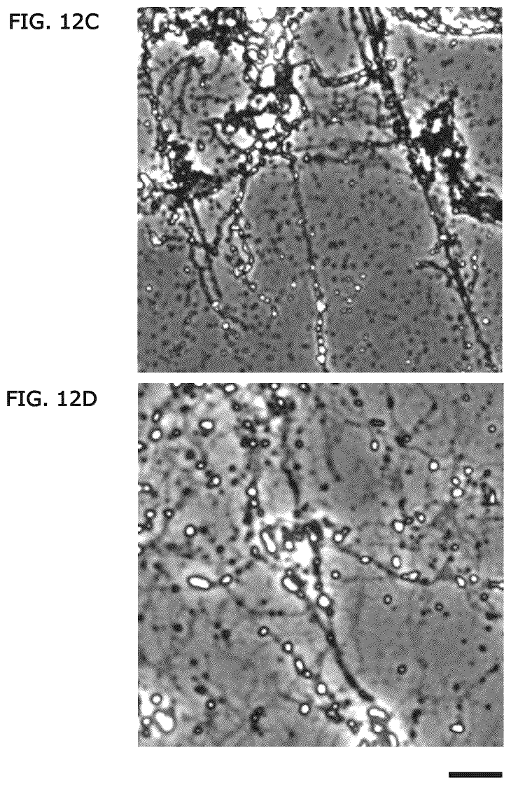

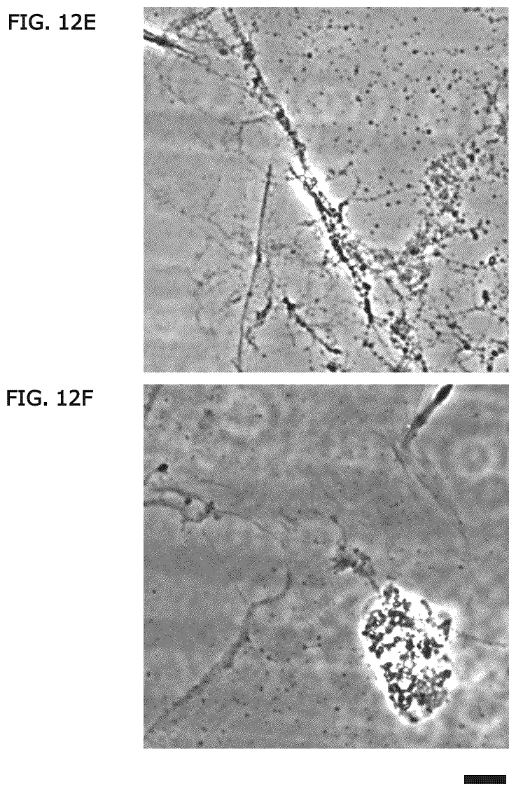

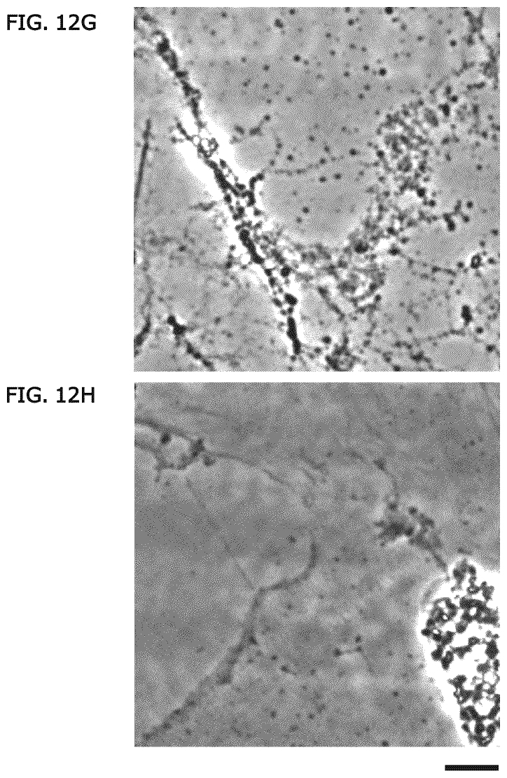

FIG. 12A and FIG. 12C show micrographs of a fixed supernatant sample with H&E stain. FIG. 12B and FIG. 12D show micrographs of a fixed pellet resuspension sample with H&E stain.

FIG. 12E and FIG. 12G show micrographs of a fixed supernatant sample with alcian blue stain. FIG. 12F and FIG. 12H show micrographs of a fixed pellet resuspension sample with alcian blue stain.

Example 10. Detecting extracellular matrix bodies isolated from bodily fluid by differential centrifugation at 5,000 g. A BVH sample was placed into a centrifugation tube and spun at 5,000 g for 10 minutes at 4° C. Supernatant was collected and the pellet resuspended in 200 μl of PBS. Samples of the supernatant and pellet resuspension were fixed by EDC-glutaraldehyde fixation on glass slides for microscopy. Image analysis showed that 50% of the extracellular matrix bodies were found in the supernatant and 50% were found in the pellet.

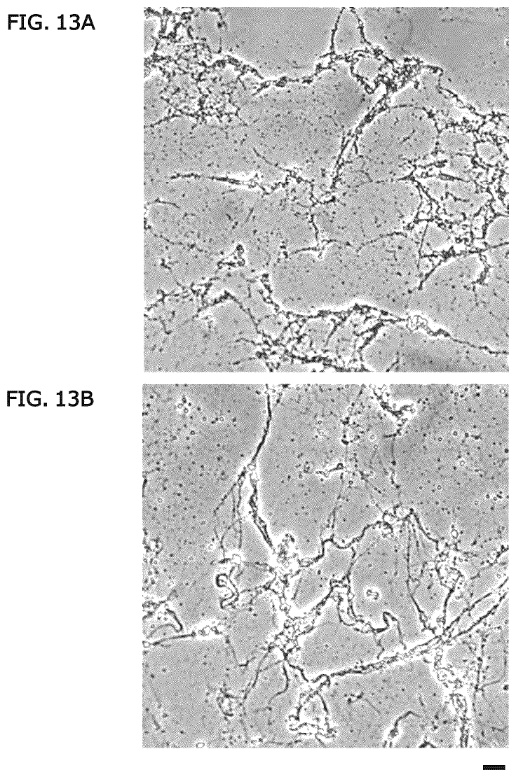

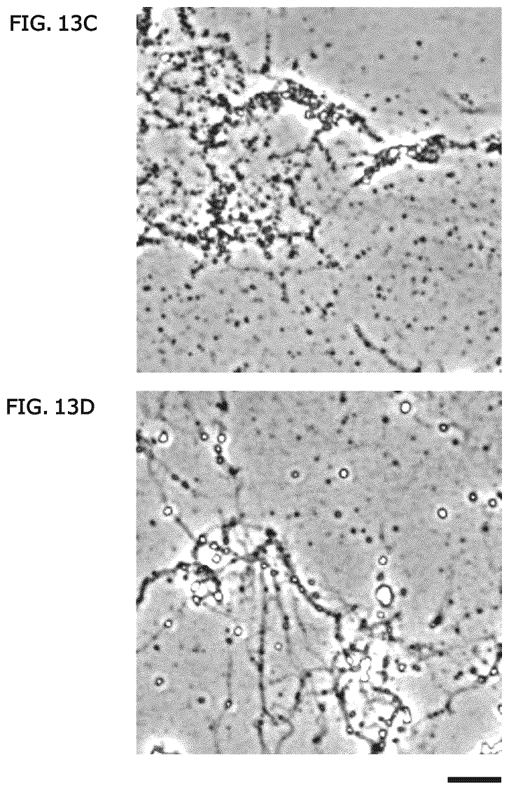

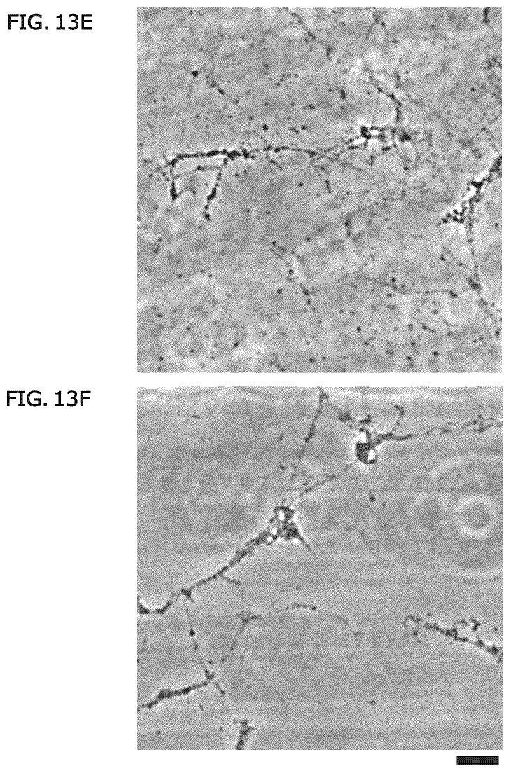

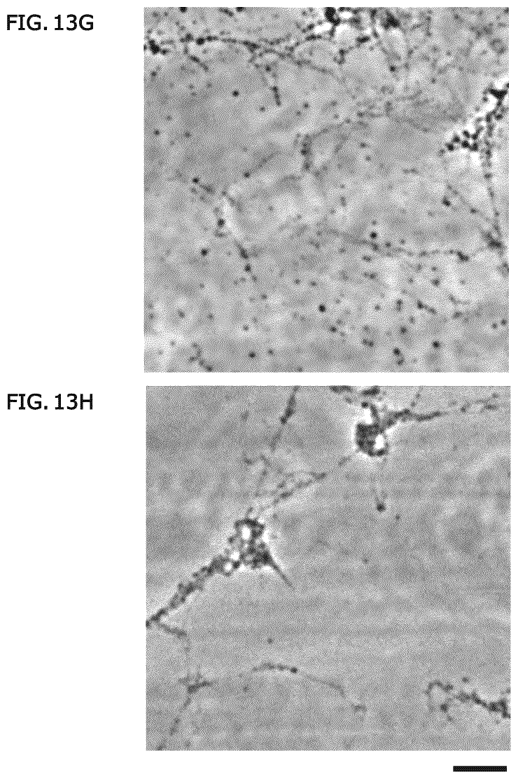

FIG. 13A and FIG. 13C show micrographs of a fixed supernatant sample with H&E stain. FIG. 13B and FIG. 13D show micrographs of a fixed pellet resuspension sample with H&E stain.

FIG. 13E and FIG. 13G show micrographs of a fixed supernatant sample with alcian blue stain. FIG. 13F and FIG. 13H show micrographs of a fixed pellet resuspension sample with alcian blue stain.

Example 11. Detecting extracellular matrix bodies isolated from bodily fluid by differential centrifugation at 10,000 g. A BVH sample was placed into a centrifugation tube and spun at 10,000 g for 10 minutes at 4° C. Supernatant was collected and the pellet resuspended in 200 μl of PBS. Samples of the supernatant and pellet resuspension were fixed by EDC-glutaraldehyde fixation on glass slides for microscopy. Image analysis showed that 40% of the extracellular matrix bodies were found in the supernatant and 60% were found in the pellet.

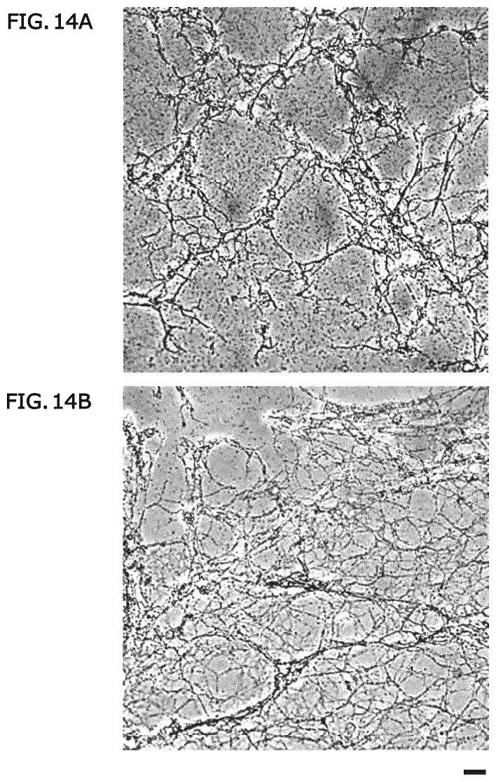

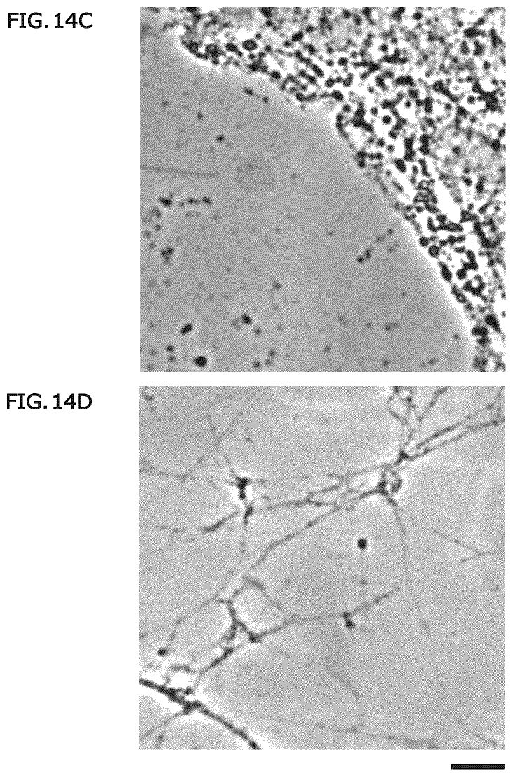

FIG. 14A shows a micrograph of a fixed supernatant sample with H&E stain. FIG. 14B shows a micrograph of a fixed pellet resuspension sample with H&E stain.

FIG. 14C shows a micrograph of a fixed supernatant sample with alcian blue stain. FIG. 14D shows a micrograph of a fixed pellet resuspension sample with alcian blue stain.

Example 12. Detecting extracellular matrix bodies isolated from bodily fluid by differential centrifugation at 12,000 g. A BVH sample was placed into a centrifugation tube and spun at 12,000 g for 10 minutes at 4° C. Supernatant was collected and the pellet resuspended in 200 μl of PBS. Samples of the supernatant and pellet resuspension were fixed by EDC-glutaraldehyde fixation on glass slides for microscopy. Image analysis showed that 20% of the extracellular matrix bodies were found in the supernatant and 80% were found in the pellet.

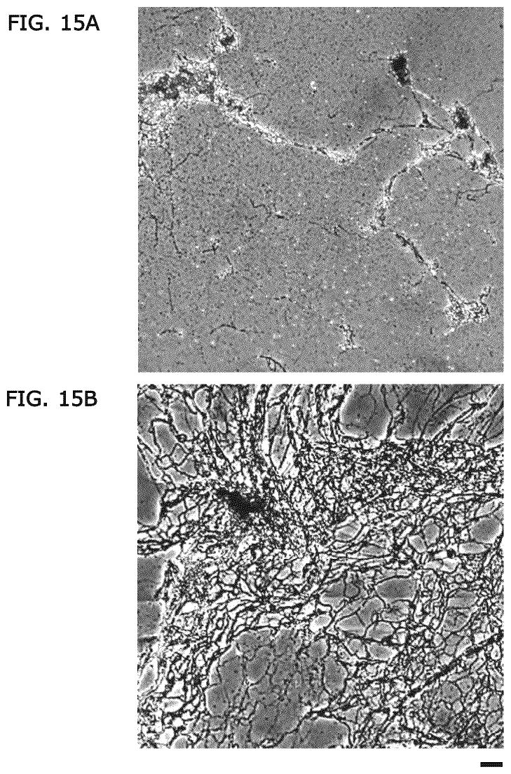

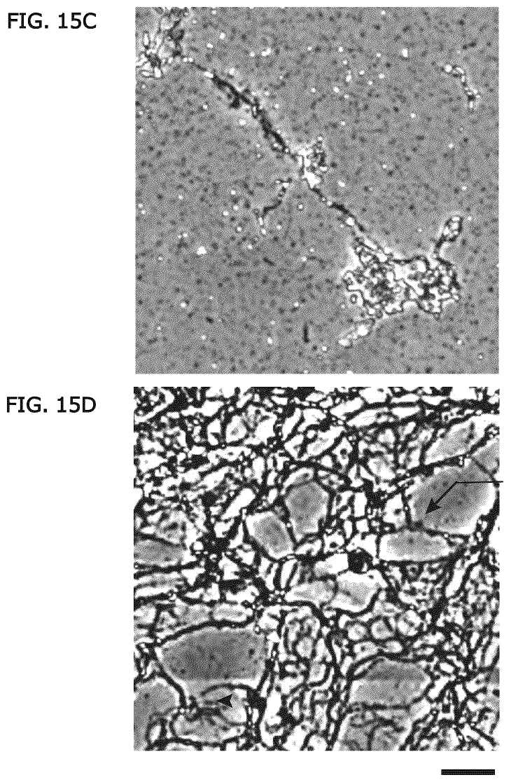

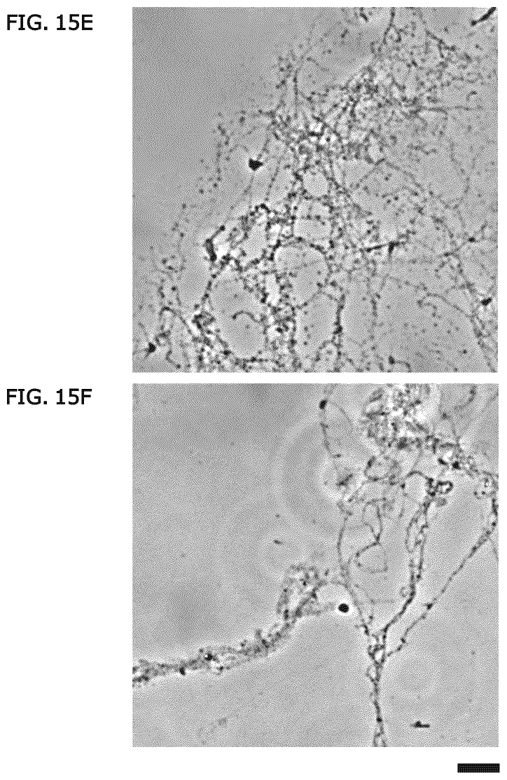

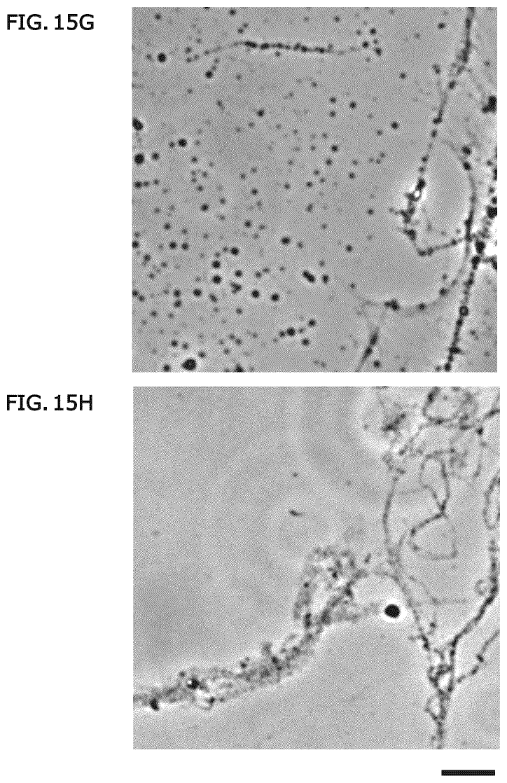

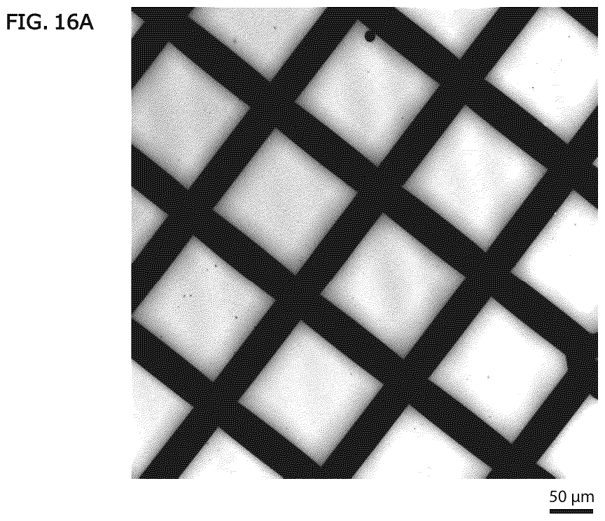

FIG. 15A and FIG. 15C show micrographs of a fixed supernatant sample with H&E stain. FIG. 15B and FIG. 15D show micrographs of a fixed pellet resuspension sample with H&E stain.

FIG. 15E and FIG. 15G show micrographs of a fixed supernatant sample with alcian blue stain. FIG. 15F and FIG. 15H show micrographs of a fixed pellet resuspension sample with alcian blue stain.

Example 13. Preparation of bovine vitreous humor bodily fluid. Bovine eyes for dissection were placed in a 100 mm plastic petri dish on ice to prevent nucleic acid and protein degradation. Using an SZX-16 stereo dissecting microscope (Olympus), orbital fat and extraocular muscles attached to the globe were removed. The globe was rinsed with 5 ml ice-cold Tris Buffered Saline (TBS) containing 50 mM Tris-HCl, 150 mM NaCl (pH 8.0) for 1 minute at 4° C. Vitreous was dissected by making an sclerotomy incision 4 mm posterior to the limbus using a 16 g needle and then making a circumferential sagittal incision with scissors to separate the globe into an anterior and posterior cup. Scissors were used to cut and remove the formed vitreous and to sever adhesions between vitreous and ocular structures. Vitreous contamination by uveal tissue or neural retina was avoided. Vitreous samples were rinsed with TBS (pH 8.0) for 1 min at 4° C. Vitreous specimens collected were placed in 1.5 ml centrifuge tubes frozen at −80° C. until use.

Example 14. Preparation of human plasma bodily fluid. Human plasma samples collected in Streck ct DNA blood collection tubes were used (PrecisionMed, Inc., Solana Beach). For these samples, whole blood was drawn into Streck blood collection tubes, and the sample was inverted 8-10 times to mix the blood with anticoagulant. Within 4 hours of collection, the sample was centrifuged at room temperature at 1,200 g for 10 minutes to isolate the plasma. The supernatant was aspirated and divided into new tubes. The plasma tubes were centrifuged at 1,200 g for 10 minutes at room temperature, and 1.0 mL aliquots of the supernatant were transferred into cryovials and placed on ice. The specimens were stored at −70° C. or colder prior to shipping.

Example 15. Preparation of human aqueous humor bodily fluid. Samples of aqueous humor were obtained with informed consent during standard therapeutic cataract surgery. At the beginning of the cataract surgery a 30-gauge needle on a TB syringe was inserted through the clear cornea to aspirate 0.05-0.1 cc of aqueous humor. Samples were transported on ice.

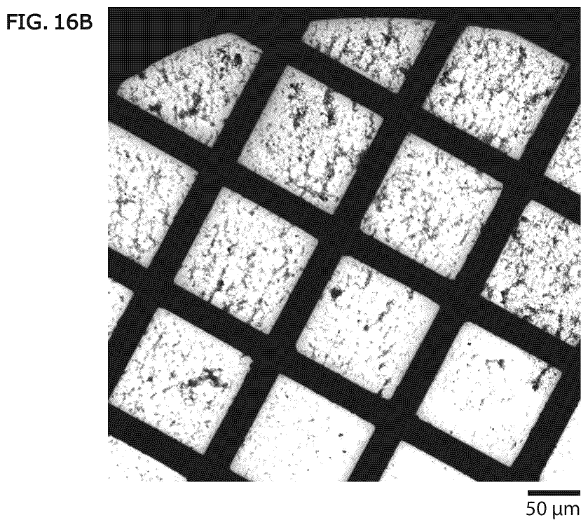

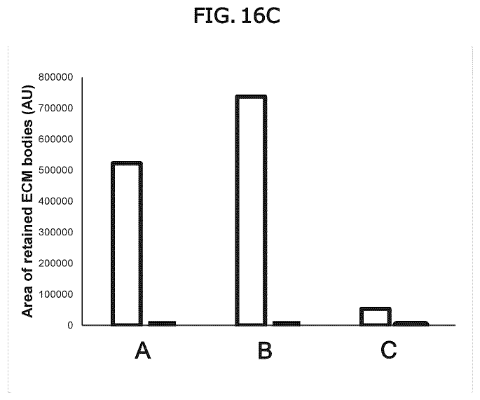

Example 16. Extracellular matrix bodies were not detected in bodily fluid by conventional aldehyde fixation. Extracellular matrix bodies were not consistently detected in human aqueous humor using conventional glutaraldehyde fixation techniques with TEM and negative staining with uranyl acetate. In this example, an aliquot of aqueous humor biofluid was applied to an electron microscopy grid and followed with standard protocols for glutaraldehyde fixation and negative staining with uranyl acetate. As shown in FIG. 16A, imaging with TEM did not detect extracellular matrix bodies.

Example 17. Extracellular matrix bodies were surprisingly detected in bodily fluid by carbodiimide fixation. Extracellular matrix bodies were detected in human aqueous humor using carbodiimide fixation. EDC is a heat stable carbodiimide fixative that creates a non-reversible crosslink between positively charged amino group side chains and carboxyl groups of proteins. Equal amounts of aqueous humor and EDC fixation solution were applied to the surface of a poly-l-lysine coated formvar TEM grid. The EDC fixative was activated at 50° C. for 3 hr, then removed, and a glutaraldehyde fixation solution was applied, followed by washing and negative staining. As shown in FIG. 16B, imaging with TEM detected abundant extracellular matrix bodies.

Analysis showed that from about 10-fold up to about 4000-fold more extracellular matrix bodies were observed by EDC fixation as compared to conventional glutaraldehyde-only fixation. FIG. 16C shows a comparison of the quantity of extracellular matrix bodies counted in images of three native human aqueous humor samples (A, B, C) obtained by the carbodiimide-fixation method of this invention (white bar) as compared to conventional fixation (solid bar). The detected quantities of extracellular matrix bodies are surprisingly enhanced by this invention.

Example 18. Detecting extracellular matrix bodies in human plasma by carbodiimide fixation. Extracellular matrix bodies were detected in bodily fluid by carbodiimide fixation. Extracellular matrix bodies in biological fluids were immobilized, stained, and imaged on glass slides by non-reversible crosslinking with the EDC-ETT solution.

An EDC-ETT solution was prepared. 1-Methylimidazole buffer solution (0.1 M 1-methylimidazole, 300 mM NaCl, with pH adjusted to 8.0 with 12 N NaOH) and stored the solution for up to 3 months at room temperature. EDC solution was freshly prepared for each experiment. We measured 0.96 ml the 1-Methylimidazole buffer solution and added 13 mg of 5-(Ethylthio)-1H-tetrazole (ETT, Sigma Aldrich) to a final concentration of 0.1 M. The pH was adjusted to 8.0 with 12 N NaOH. 19.2 mg of 1-ethyl-3-(3-dimethylaminopropyl) carbodiimide (EDC) was added (Sigma Aldrich) to a final concentration of 0.10 M, and readjusted the pH to 8.0 using 12 M HCl. The EDC-ETT solution was placed on ice until use.

SuperFrost Plus glass slides (Fisher Scientific) were rinsed with 10 ml of deionized (DI) water and allowed to air dry. A 40 cm² square border was demarked with clear nail polish and dried for 15 minutes at room temperature. A stock solution of poly-l-lysine was prepared using DI water at a ratio of 1:2 (poly-l-lysine to DI water) and equilibrated at room temperature. 200 μl of diluted poly-l-lysine solution was applied to the square border and incubated for 5 minutes at room temperature. The diluted poly-l-lysine solution was removed by pipet or decanting and the slides dried overnight.

0.013 grams of 5-ETT was mixed with 0.96 ml of 0.1 M 1-Methylimidazole buffer (pH 8.0) in a 1.5 ml tube Eppendorf tube. 0.0192 g of EDC-HCl was added. pH was adjusted to 8.0.

Equal volumes of a human plasma sample and a cold EDC-ETT solution were added to a 1.5 ml Eppendorf siliconized tube, and a sample of the mixture was applied to an ice-chilled slide and incubated cold for 30 minutes. The slide was then incubated overnight at 37° C., and the EDC-ETT solution was removed. The slide was washed twice with TBS 1× for 5 minutes at room temperature, and then stained with Hematoxylin and Eosin staining (Vector Labs, H-3502, Burlingame).

Extracellular matrix bodies were stained with hematoxylin and eosin to mark nuclei (blue color) and cytoplasm/extracellular matrix (pink), respectively, using an Hematoxylin and Eosin Stain Kit (Vector Labs). Hematoxylin stain was applied to the slide and incubated for 5 minutes at room temperature. Excess staining solution was removed by decanting and the sample was washed with distilled water. Bluing Reagent was applied to the slide and incubated for 15 seconds, and the wash step was repeated twice. The stained area was rinsed with 100% ethanol and covered with Eosin Y solution for 3 minutes at room temperature and rinsed with ethanol. 200 μl of 80% glycerol was added to preserve the specimen. A coverslip was placed on top of the sample and the edges sealed with nail polish.

Extracellular matrix bodies were stained with alcian blue for detecting glycosaminoglycans and hyaluronic acids. A sample of bodily fluid was fixed to a poly-L-lysine coated Superfrost Plus glass slide using EDC-crosslinking as described above. 1% Alcian Blue in 3% acetic acid was applied to a demarked region, incubated in a dark chamber for 30 minutes, and rinsed with distilled water at room temperature. The solution was removed with a pipette or decanted. Samples were washed with acidified aqueous wash (3% glacial acetic acid diluted in deionized water) at room temperature. The demarked region was coved with a 40% glycerol solution and an adequate amount of mounting medium and covered with a cover slip. The cover slip edges were sealed with nail polish. The slide was dried for 15 minutes at room temperature prior to imaging. A Zeiss Axiovert 200 wide-field microscope with Zen imaging software was used to capture images within 1-2 hours of Alcian blue/PSR staining on the glass slide.

Example 19. Isolating extracellular matrix bodies in bodily fluid by serial centrifugation. Extracellular matrix bodies were isolated from bovine vitreous humor by serial centrifugation. Bovine vitreous humor was obtained as described in Example 1. 700-800 μl samples were placed in 1 mL centrifuge tubes and spun at each of 500, 1,000, 2,000, 3,000, 4,000, 5,000, 10,000 and 12,000 g for 10 minutes at 4° C. For each speed after 500 g, the supernatant was collected by aspiration and used in the next step. A sample of each of the supernatant and pellet was collected and kept at 4° C. until particle analysis by EDC-glutaraldehyde fixation on glass slides.

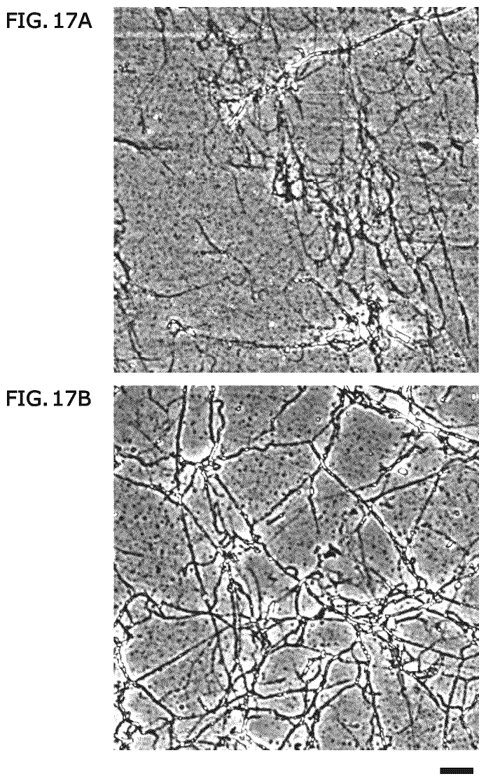

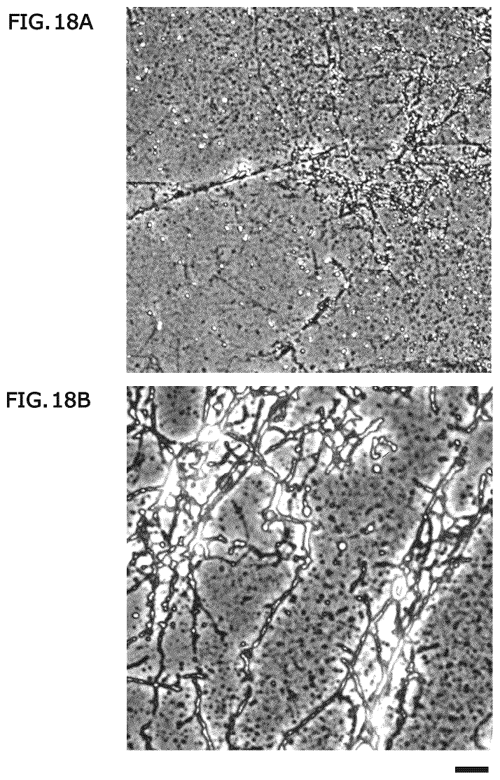

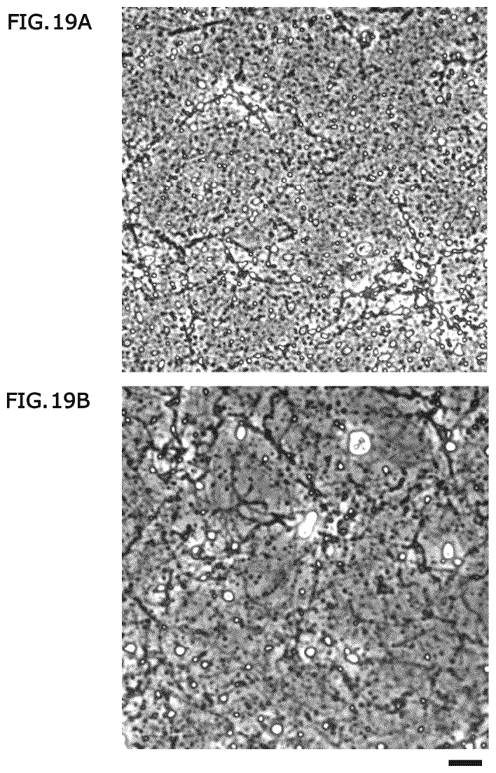

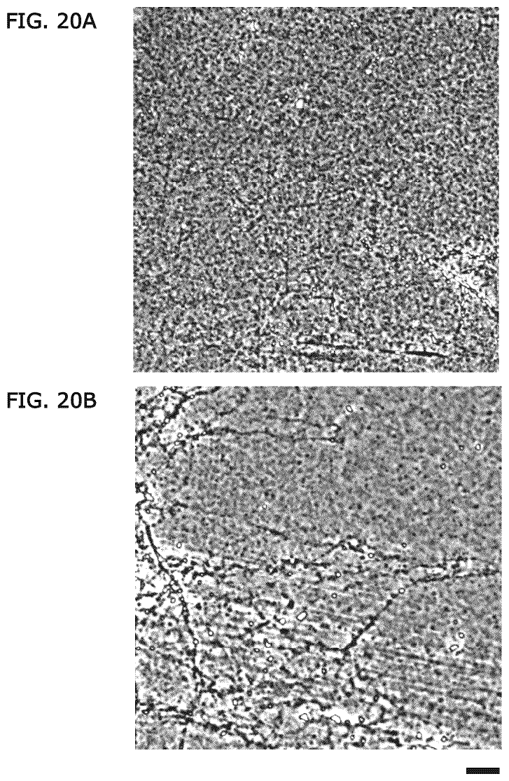

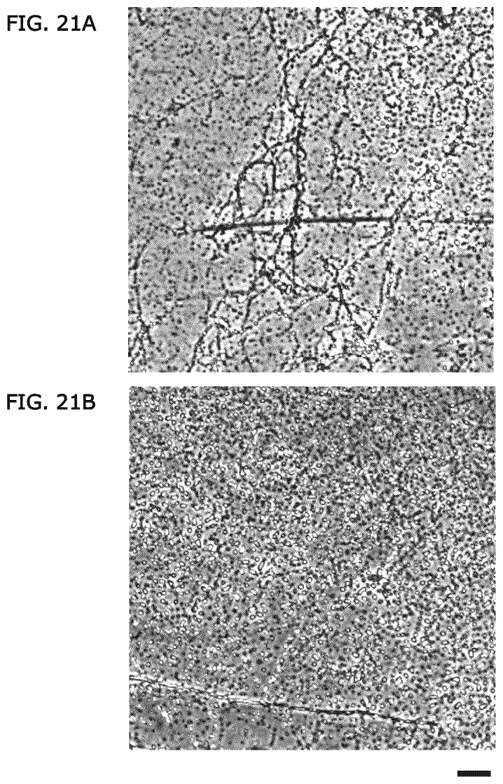

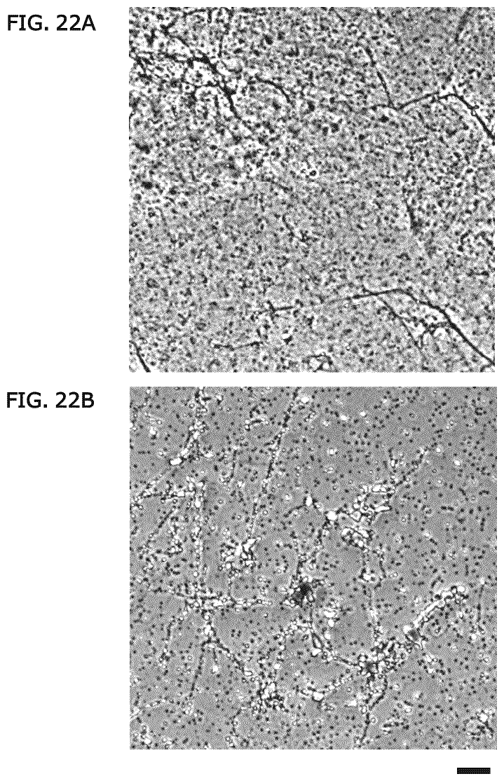

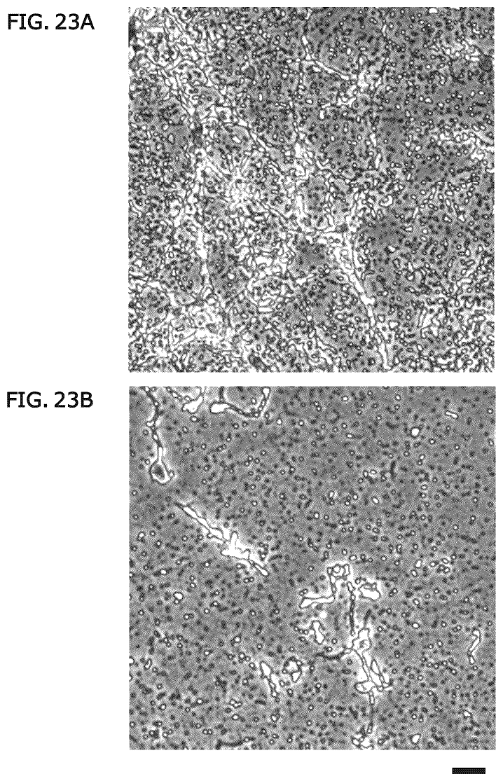

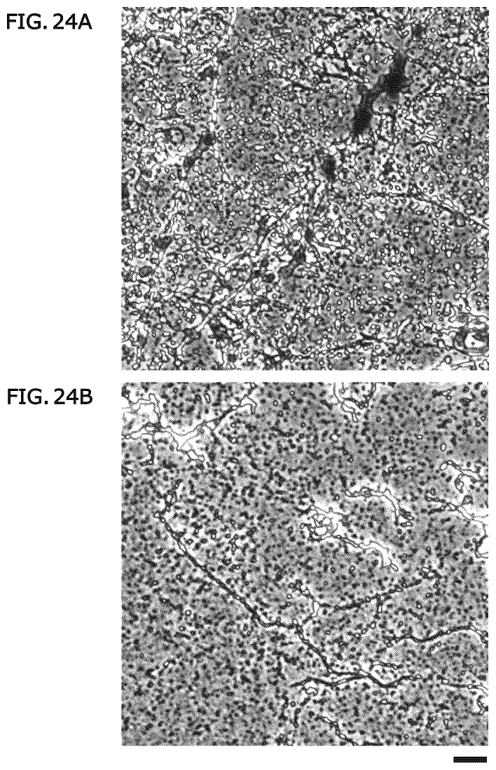

FIGS. 17 through 24 show representative micrographs of extracellular matrix bodies isolated in bovine vitreous humor by serial centrifugation. These images show extracellular matrix bodies in the supernatant and pellet after centrifugation. Images were obtained by light microscopy of a sample fixed with EDC on a glass slide and stained with hematoxylin and eosin.

Example 20. Particle analysis of isolated extracellular matrix bodies obtained from bodily fluid by differential centrifugation. Extracellular matrix bodies were isolated from bovine vitreous humor by serial centrifugation. Bovine vitreous humor was obtained as described in Example 1. 700-800 μl samples were placed in 1 mL centrifuge tubes and spun at each of 500, 1,000, 2,000, 3,000, 4,000, 5,000, 10,000 and 12,000 g for 10 minutes at 4° C. For each speed after 500 g, the supernatant was collected by aspiration and used in the next step. A sample of each of the supernatant and resuspended pellet was collected and kept at 4° C. until particle analysis by EDC-glutaraldehyde fixation on glass slides.

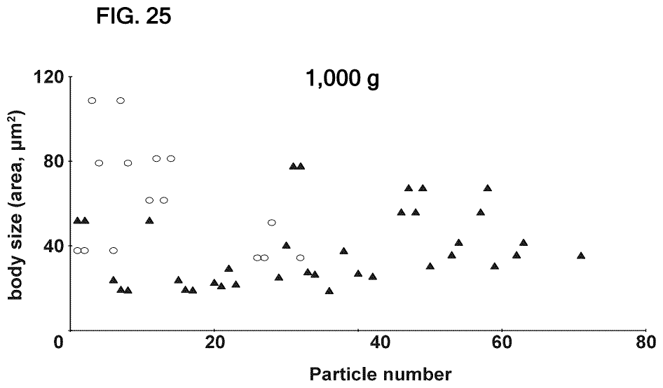

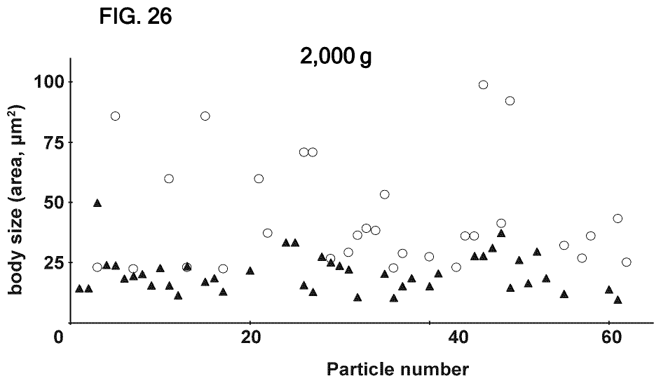

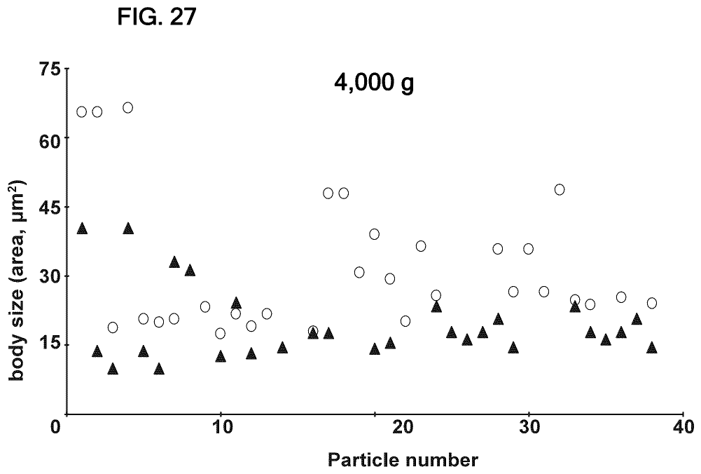

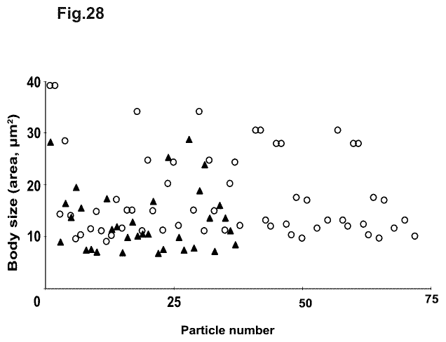

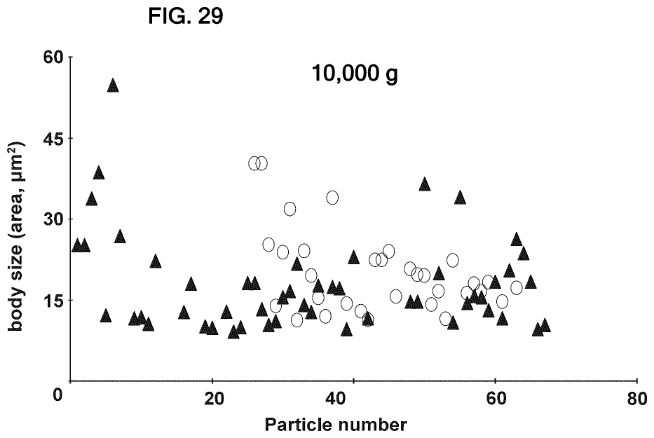

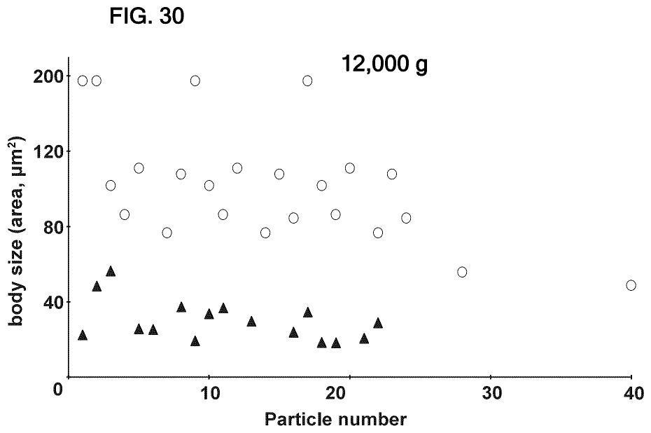

FIGS. 25 through 30 show particle analysis graphs of the extracellular matrix bodies isolated from bovine vitreous humor by serial centrifugation.

Example 21. Isolating extracellular matrix bodies from bodily fluid by differential centrifugation combined with membrane filtration. Extracellular matrix bodies were isolated from bovine vitreous humor by serial centrifugation. Bovine vitreous humor was obtained as described in Example 1.

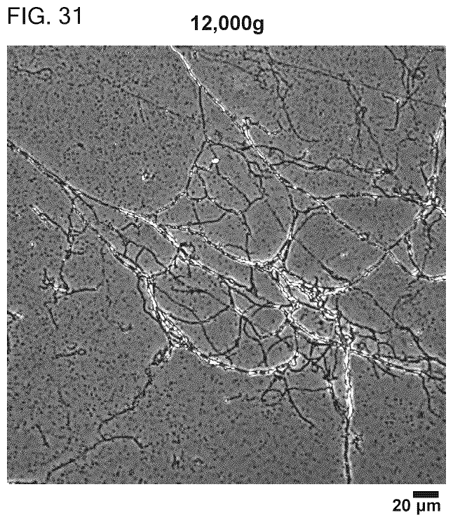

FIG. 31 shows a representative micrograph of bovine vitreous humor processed with centrifugation. The bovine vitreous humor was centrifuged at 12,000 g, followed by resuspending the pellet in 400 μl of phosphate buffer saline. This image showed that significant amounts of extracellular matrix bodies were obtained from the resuspended pellet. Image was obtained by light microscopy of a sample fixed with EDC on a glass slide and stained with hematoxylin and eosin.

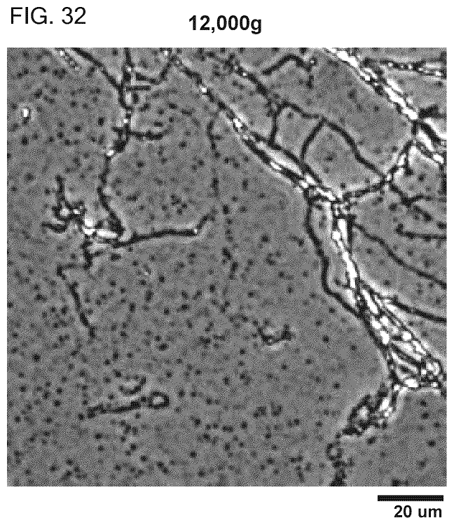

FIG. 32 shows an expansion of the micrograph in FIG. 31.

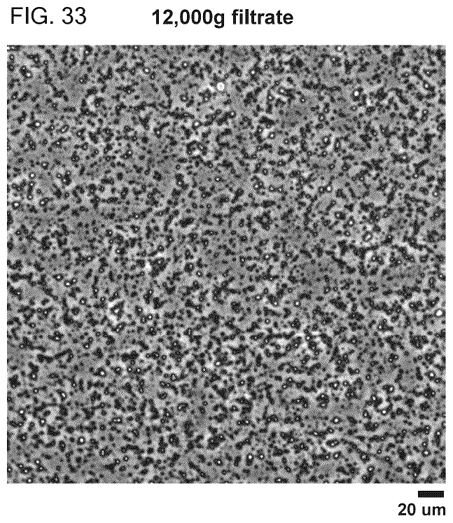

FIG. 33 shows a representative micrograph of bovine vitreous humor processed with centrifugation and membrane filtration. The bovine vitreous humor was centrifuged at 12,000 g, followed by resuspending the pellet in 400 μl of phosphate buffer saline and filtering with a cellulose acetate membrane with a 0.45 μm pore size. This image showed that substantially all extracellular matrix bodies were removed from the filtrate fraction distal to the 0.45 μm membrane. Image was obtained by light microscopy of a sample fixed with EDC on a glass slide and stained with hematoxylin and eosin.

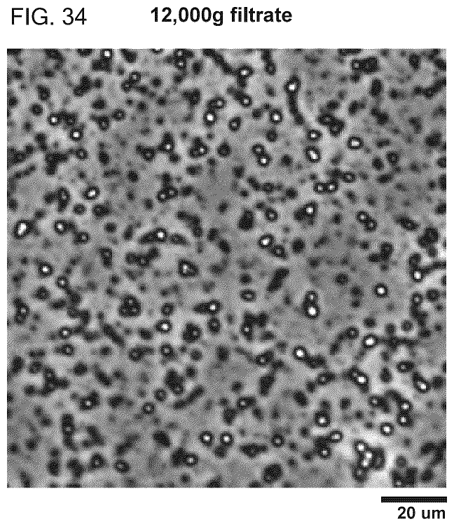

FIG. 34 shows an expansion of the micrograph in FIG. 33.

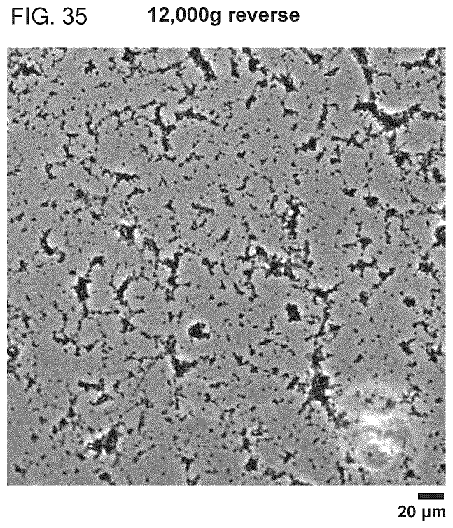

FIG. 35 shows a representative micrograph of bovine vitreous humor extracted from the filter after processing with centrifugation and membrane filtration. The bovine vitreous humor was centrifuged at 12,000 g, followed by resuspending the pellet in 400 μl of phosphate buffer saline and filtering the suspension with a cellulose acetate membrane with a 0.45 μm pore size. Extracellular matrix bodies we recovered by extracting the material held on the proximal surface of the 0.45-micron filter. This image shows the presence of extracellular matrix bodies extracted from proximal surface of the 0.45-micron filter.

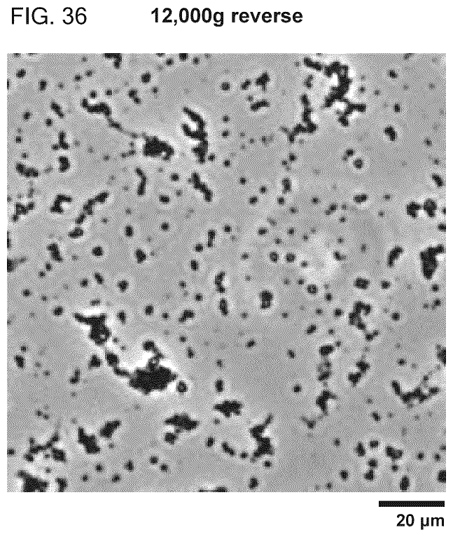

FIG. 36 shows an expansion of the micrograph in FIG. 35.

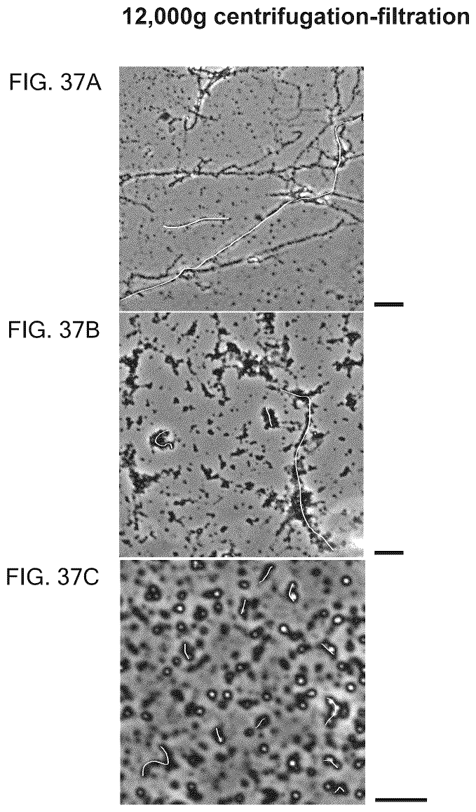

FIG. 37A shows an embodiment of a method for quantifying extracellular matrix bodies in a bodily fluid, bovine vitreous humor. The lengths of extracellular matrix bodies were measured by segmenting the image to identify and measure uninterrupted lengths of the extracellular matrix bodies (white lines).

FIG. 37B shows an embodiment of a method for quantifying extracellular matrix bodies in a bodily fluid, bovine vitreous humor. The lengths of extracellular matrix bodies can be measured by segmenting the image to identify and measure uninterrupted lengths of the extracellular matrix bodies (white lines).

FIG. 37C shows an embodiment of a method for quantifying extracellular matrix bodies in a bodily fluid, bovine vitreous humor. The lengths of extracellular matrix bodies can be measured by segmenting the image to identify and measure uninterrupted lengths of the extracellular matrix bodies (white lines).

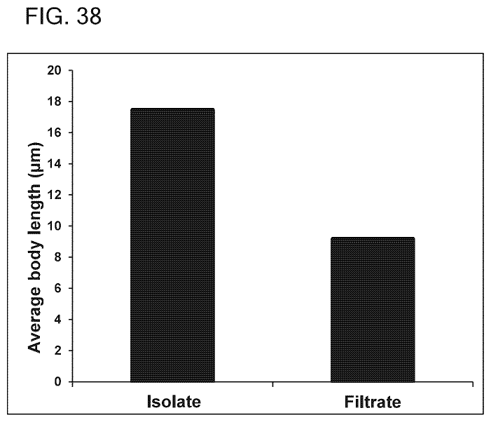

FIG. 38 shows quantification of extracellular matrix bodies in a method of this invention. Bovine vitreous humor was centrifuged at 12,000 g, followed by resuspending the pellet in 400 μl of phosphate buffer saline and filtering the suspension with a cellulose acetate membrane with a 0.45 μm pore size. Extracellular matrix bodies we recovered by extracting the material held on the proximal surface of the 0.45-micron filter. The extracted isolate contained 65% of the extracellular matrix bodies. The filtrate contained 35% of the extracellular matrix bodies.

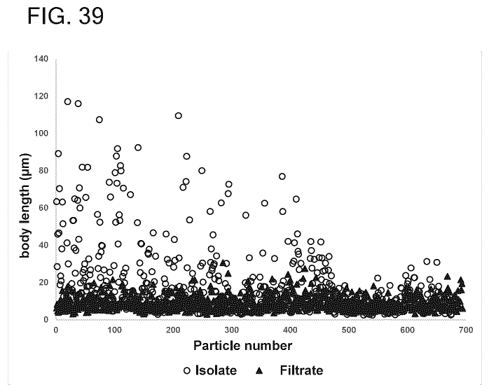

FIG. 39 shows quantification of extracellular matrix bodies in a method of this invention. Bovine vitreous humor was centrifuged at 12,000 g, followed by resuspending the pellet in 400 μl of phosphate buffer saline and filtering the suspension with a cellulose acetate membrane with a 0.45 μm pore size. Extracellular matrix bodies we recovered by extracting the material held on the proximal surface of the 0.45-micron filter. The extracted isolate extracellular matrix bodies (open circles) were larger in average body length than extracellular matrix bodies obtained from the filtrate (filled triangles).

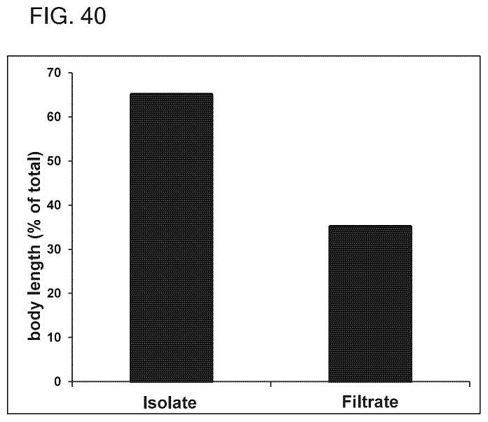

FIG. 40 shows quantification of extracellular matrix bodies in a method of this invention. Bovine vitreous humor was centrifuged at 12,000 g, followed by resuspending the pellet in 400 μl of phosphate buffer saline and filtering the suspension with a cellulose acetate membrane with a 0.45 μm pore size. Extracellular matrix bodies we recovered by extracting the material held on the proximal surface of the 0.45-micron filter. The extracted isolate extracellular matrix bodies (open circles) comprised significantly greater total body length than extracellular matrix bodies obtained from the filtrate (filled triangles).

Example 22. Isolating extracellular matrix bodies from bodily fluid by single pass membrane filtration. Extracellular matrix bodies were isolated from bovine vitreous humor by membrane filtration. Bovine vitreous humor was obtained as described in Example 1.

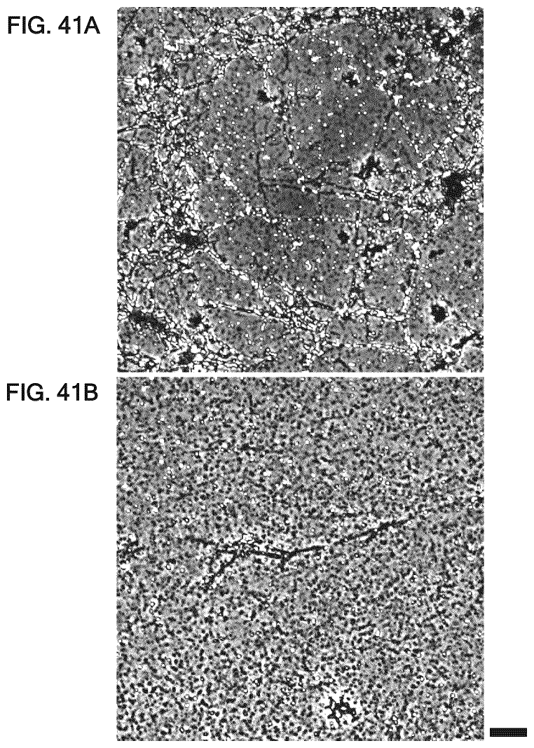

FIG. 41A shows a representative photomicrographic of the enrichment of extracellular matrix bodies by filtration from a biological fluid, bovine vitreous humor, using a cellulose acetate membrane filter with a pore size of 0.22 micron filter. Extracellular matrix bodies were recovered by reversing the filter and extracting material from the proximal side of the filter to obtain an isolate. Image was taken on a glass slide with EDC fixation. Scale bars are 20 um.

FIG. 41B shows a representative photomicrograph of the filtrate from a biological fluid, bovine vitreous humor, using a cellulose acetate membrane filter with a pore size of 0.22 micron filter. Relatively few extracellular matrix bodies, if any, were found in the filtrate. Scale bars are 20 μm.

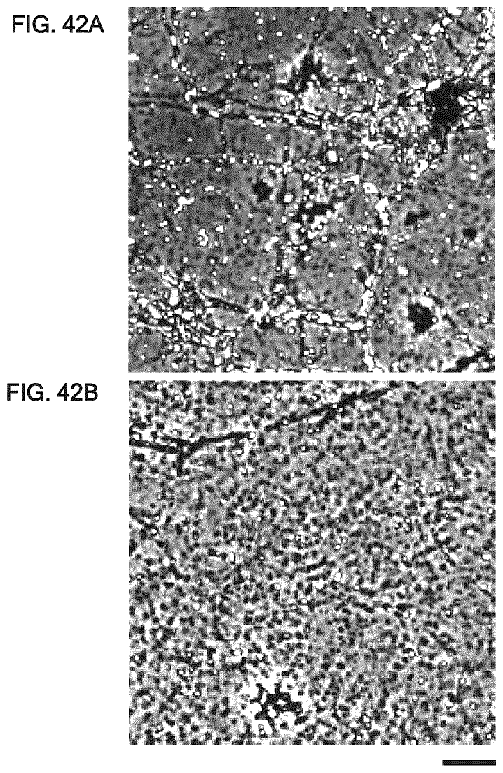

FIG. 42A shows an expansion of the micrograph in FIG. 41A.

FIG. 42B shows an expansion of the micrograph in FIG. 41B.

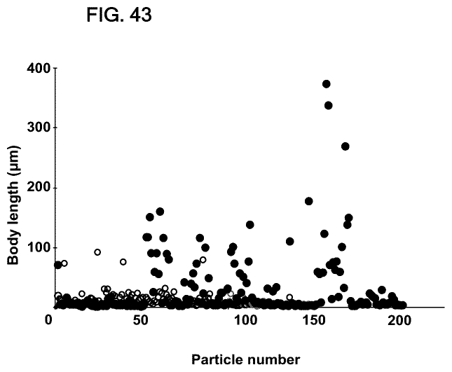

FIG. 43 shows a representative photomicrographic of the enrichment of extracellular matrix bodies by filtration from a biological fluid, bovine vitreous humor, using a cellulose acetate membrane filter with a pore size of 0.22 micron filter. Extracellular matrix bodies were recovered by reversing the filter and extracting material from the proximal side of the filter to obtain an isolate by reverse elution. Image was taken on a glass slide with EDC fixation. Scale bars are 20 um. A greater abundance of extracellular matrix bodies were found in the isolate (filled circles) as compared to the filtrate (open circles).

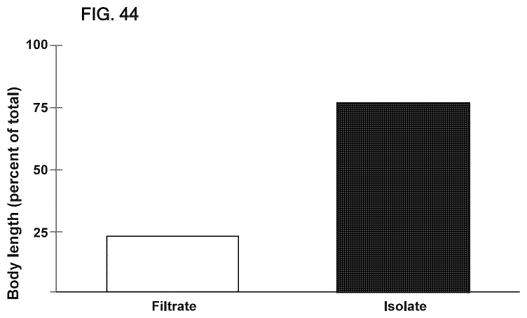

FIG. 44 shows a graphical representation of the enrichment of extracellular matrix bodies by filtration from a biological fluid, bovine vitreous humor, using a cellulose acetate membrane filter with a pore size of 0.45 micron filter. A greater abundance of extracellular matrix bodies were found in the isolate as compared to the filtrate.

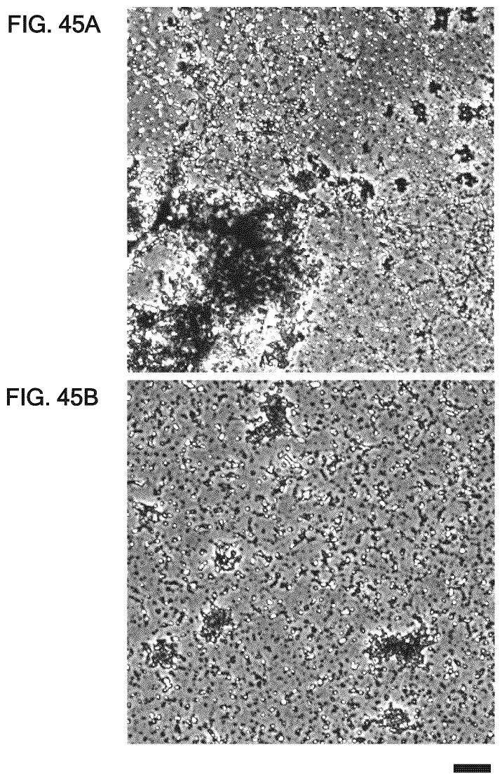

FIG. 45A shows a representative photomicrographic of the enrichment of extracellular matrix bodies by filtration from a biological fluid, bovine vitreous humor, using a cellulose acetate membrane filter with a pore size of 0.22 micron filter. Extracellular matrix bodies were recovered by reversing the filter and extracting material from the proximal side of the filter to obtain an isolate. Image was taken on a glass slide with EDC fixation. Scale bars are 20 um.

FIG. 45B shows a representative photomicrograph of the filtrate from a biological fluid, bovine vitreous humor, using a cellulose acetate membrane filter with a pore size of 0.45 micron filter. Relatively few extracellular matrix bodies, if any, were found in the filtrate. Scale bars are 20 μm.

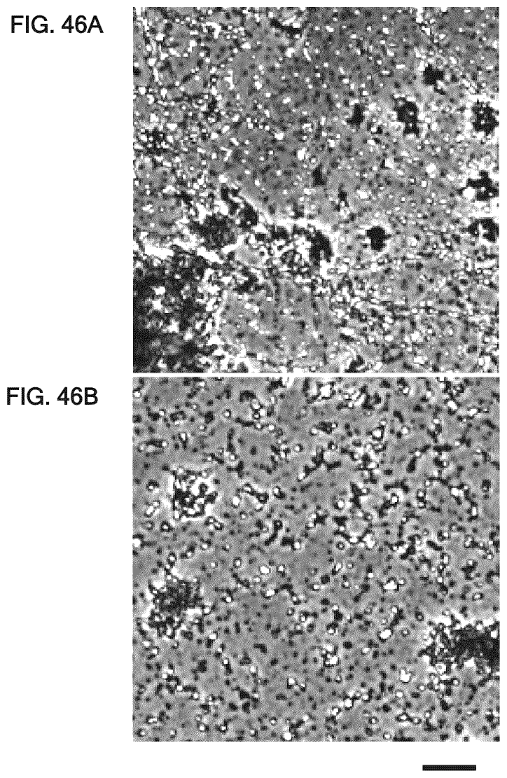

FIG. 46A shows an expansion of the micrograph in FIG. 45A.

FIG. 46B shows an expansion of the micrograph in FIG. 45B.

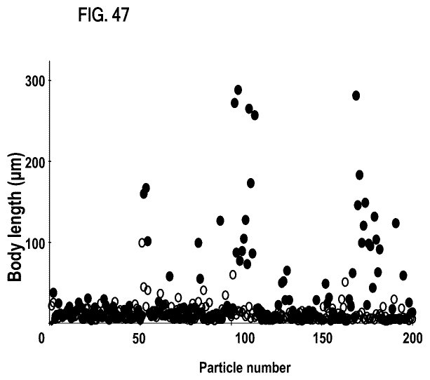

FIG. 47 shows a graphical representation of the enrichment of extracellular matrix bodies by filtration from a biological fluid, bovine vitreous humor, using a cellulose acetate membrane filter with a pore size of 0.45 micron filter. A greater abundance of extracellular matrix bodies were found in the isolate (filled circles) as compared to the filtrate (open circles). Extracellular matrix bodies were quantified by analyzing multiple photographic images (n=3). Analysis was performed in ImageJ for each 20× image. The length of extracellular matrix bodies were measured using a freehand line tool and fifty measurements were taken for each image.

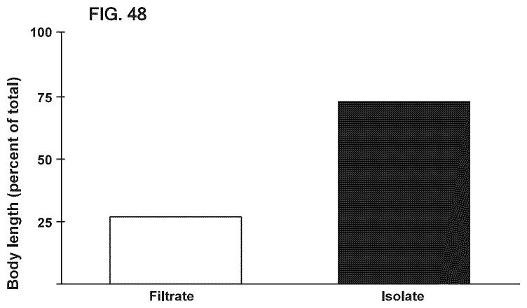

FIG. 48 shows a graphical representation of the enrichment of extracellular matrix bodies by filtration from a biological fluid, bovine vitreous humor, using a cellulose acetate membrane filter with a pore size of 0.45 micron filter. A greater abundance of extracellular matrix bodies were found in the isolate as compared to the filtrate.

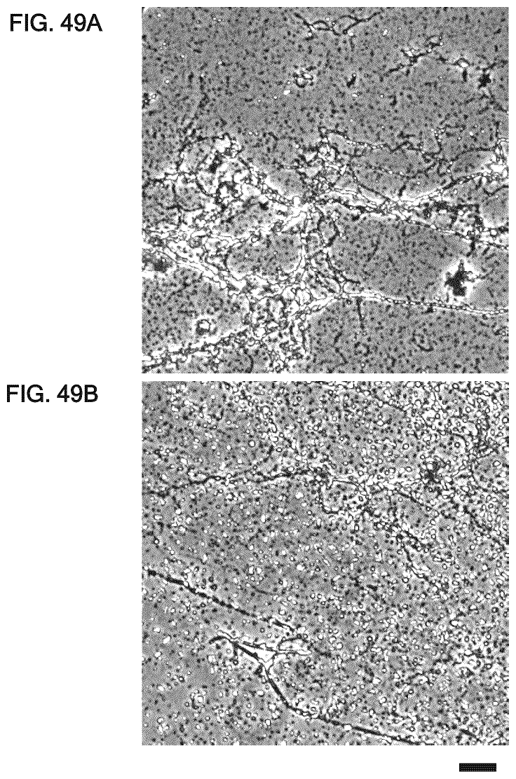

FIG. 49A shows a representative photomicrographic of the enrichment of extracellular matrix bodies by filtration from a biological fluid, bovine vitreous humor, using a cellulose acetate membrane filter with a pore size of 1 micron filter. Extracellular matrix bodies were recovered by reversing the filter and extracting material from the proximal side of the filter to obtain an isolate. Image was taken on a glass slide with EDC fixation. Scale bars are 20 um.

FIG. 49B shows a representative photomicrograph of the filtrate from a biological fluid, bovine vitreous humor, using a cellulose acetate membrane filter with a pore size of 1 micron filter. Relatively few extracellular matrix bodies, if any, were found in the filtrate. Scale bars are 20 um.

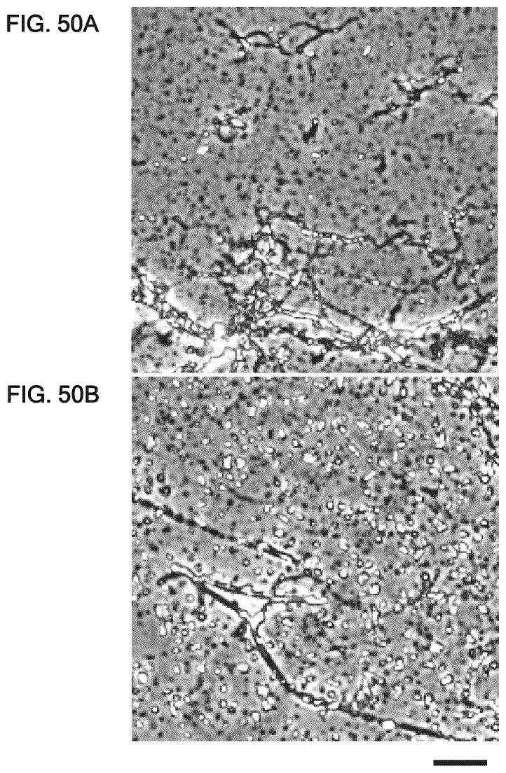

FIG. 50A shows an expansion of the micrograph in FIG. 49A.

FIG. 50B shows an expansion of the micrograph in FIG. 49B.

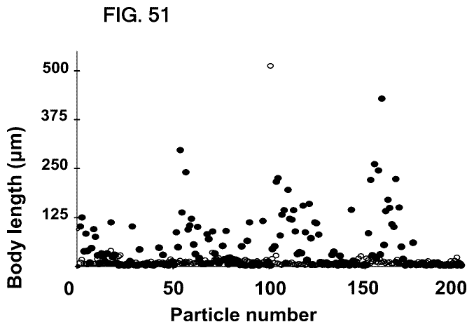

FIG. 51 shows a graphical representation of the enrichment of extracellular matrix bodies by filtration from a biological fluid, bovine vitreous humor, using a cellulose acetate membrane filter with a pore size of 1 micron filter. A greater abundance of extracellular matrix bodies were found in the isolate (filled circles) as compared to the filtrate (open circles).

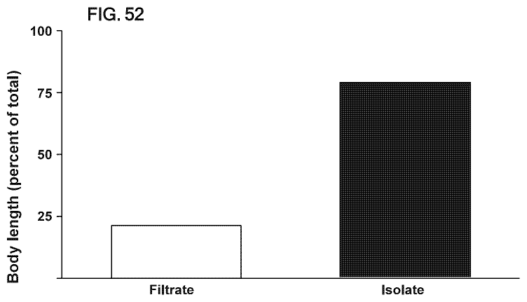

FIG. 52 shows a graphical representation of the enrichment of extracellular matrix bodies by filtration from a biological fluid, bovine vitreous humor, using a cellulose acetate membrane filter with a pore size of 1 micron filter. A greater abundance of extracellular matrix bodies were found in the isolate as compared to the filtrate.

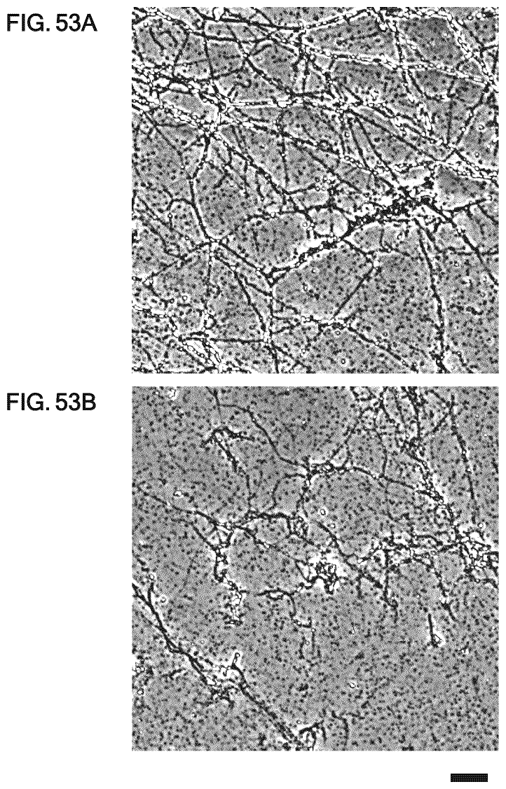

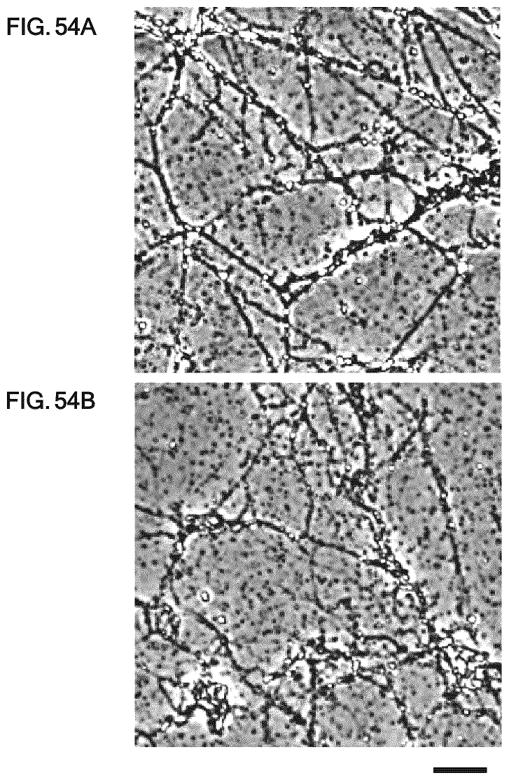

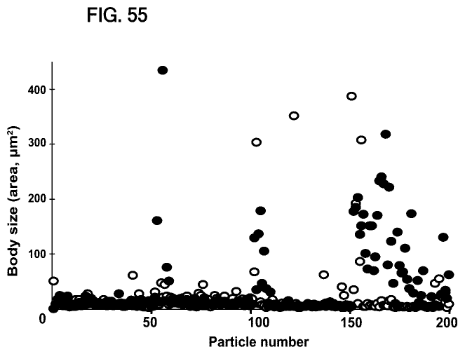

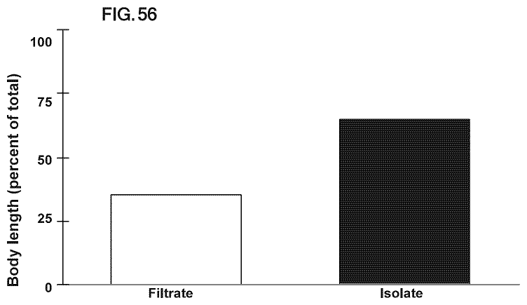

FIG. 53A shows a representative photomicrographic of the enrichment of extracellular matrix bodies by filtration from a biological fluid, bovine vitreous humor, using a cellulose acetate membrane filter with a pore size of 5 micron filter. Extracellular matrix bodies were recovered by reversing the filter and extracting material from the proximal side of the filter to obtain an isolate. Image was taken on a glass slide with EDC fixation. Scale bars are 20 um.

FIG. 53B shows a representative photomicrograph of the filtrate from a biological fluid, bovine vitreous humor, using a cellulose acetate membrane filter with a pore size of 5 micron filter. Relatively fewer extracellular matrix bodies were found in the filtrate as compared to the isolate. Scale bars are 20 um.

FIG. 50A shows an expansion of the micrograph in FIG. 49A.

FIG. 50B shows an expansion of the micrograph in FIG. 49B.

FIG. 51 shows a graphical representation of the enrichment of extracellular matrix bodies by filtration from a biological fluid, bovine vitreous humor, using a cellulose acetate membrane filter with a pore size of 1 micron filter. A greater abundance of extracellular matrix bodies were found in the isolate (filled circles) as compared to the filtrate (open circles).

FIG. 52 shows a graphical representation of the enrichment of extracellular matrix bodies by filtration from a biological fluid, bovine vitreous humor, using a cellulose acetate membrane filter with a pore size of 1 micron filter. A greater abundance of extracellular matrix bodies were found in the isolate as compared to the filtrate.

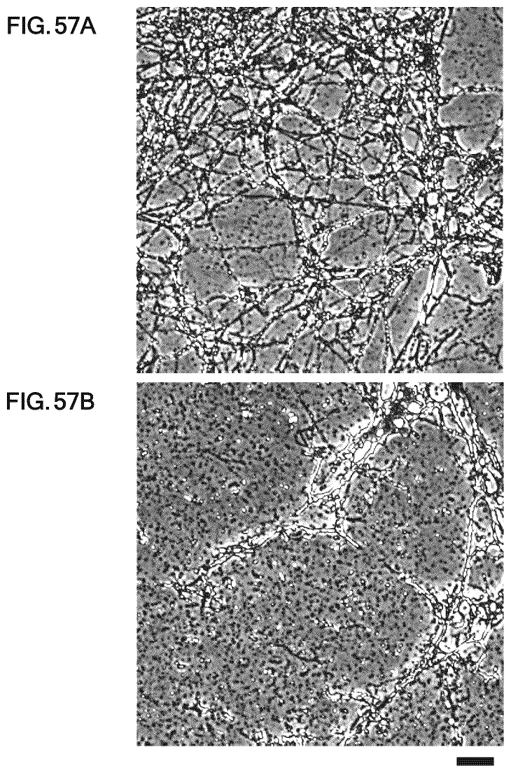

FIG. 57A shows a representative photomicrographic of the enrichment of extracellular matrix bodies by filtration from a biological fluid, bovine vitreous humor, using a cellulose acetate membrane filter with a pore size of 10 micron filter. Extracellular matrix bodies were recovered by reversing the filter and extracting material from the proximal side of the filter to obtain an isolate. Image was taken on a glass slide with EDC fixation. Scale bars are 20 um.

FIG. 57B shows a representative photomicrograph of the filtrate from a biological fluid, bovine vitreous humor, using a cellulose acetate membrane filter with a pore size of 10 micron filter. Relatively fewer extracellular matrix bodies were found in the filtrate as compared to the isolate. Scale bars are 20 um.

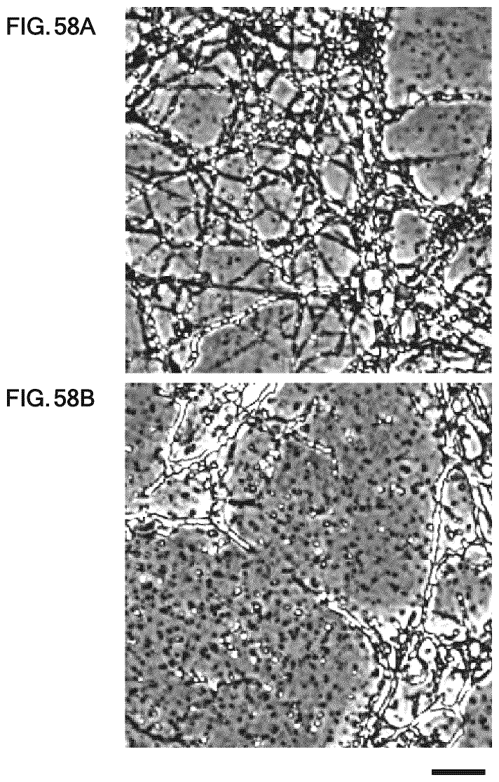

FIG. 58A shows an expansion of the micrograph in FIG. 57A.

FIG. 58B shows an expansion of the micrograph in FIG. 57B.

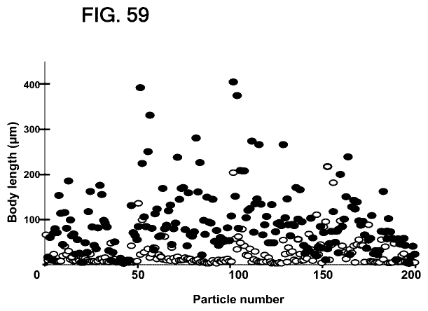

FIG. 59 shows a graphical representation of the enrichment of extracellular matrix bodies by filtration from a biological fluid, bovine vitreous humor, using a cellulose acetate membrane filter with a pore size of 10 micron filter. A greater abundance of extracellular matrix bodies were found in the isolate (filled circles) as compared to the filtrate (open circles).

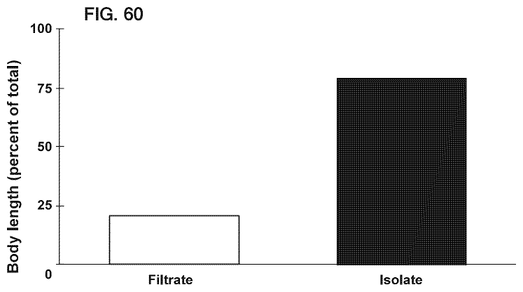

FIG. 60 shows a graphical representation of the enrichment of extracellular matrix bodies by filtration from a biological fluid, bovine vitreous humor, using a cellulose acetate membrane filter with a pore size of 10 micron filter, shown I FIG. 59. A greater abundance of extracellular matrix bodies were found in the isolate as compared to the filtrate.

Example 23. Isolating extracellular matrix bodies from bodily fluid by multiple pass serial membrane filtration. Extracellular matrix bodies were isolated from bovine vitreous humor by membrane filtration. Bovine vitreous humor was obtained as described in Example 1.

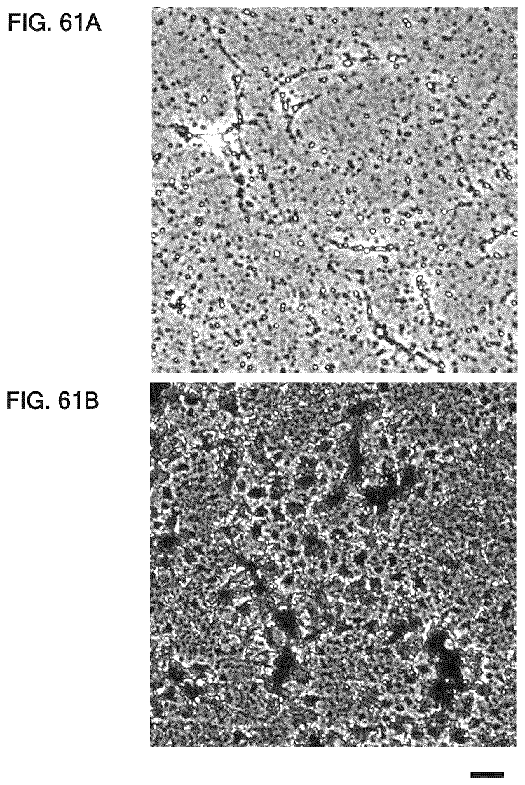

FIG. 61A shows a representative photomicrographic of the enrichment of extracellular matrix bodies by filtration from a biological fluid, bovine vitreous humor, using a cellulose acetate membrane filter with a pore size of 0.22 micron filter. Extracellular matrix bodies were recovered by reversing the filter and extracting material from the proximal side of the filter to obtain an isolate. Image was taken on a glass slide with EDC fixation. Scale bars are 20 um. This image shows the filtrate.

FIG. 61B shows a representative photomicrograph of the isolate from a biological fluid, bovine vitreous humor, using a cellulose acetate membrane filter with a pore size of 0.22 micron filter. Scale bars are 20 um. Relatively few extracellular matrix bodies, if any, were found in the isolate as compared to the filtrate.

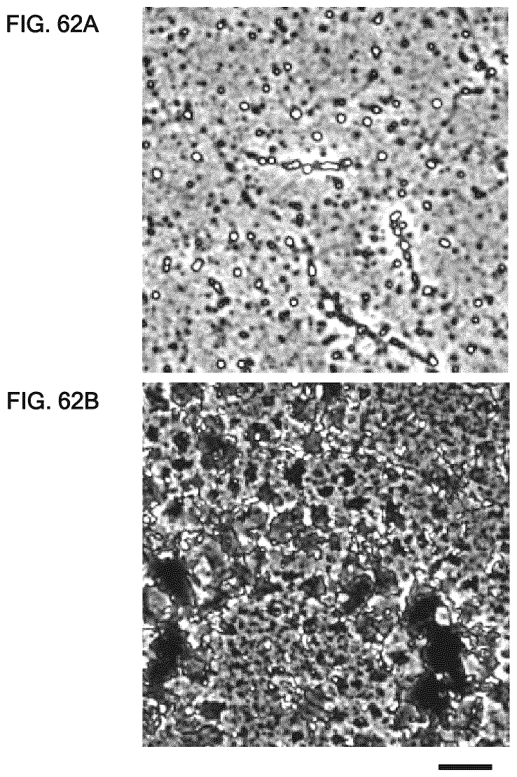

FIG. 62A shows an expansion of the micrograph in FIG. 61A.

FIG. 62B shows an expansion of the micrograph in FIG. 61B.

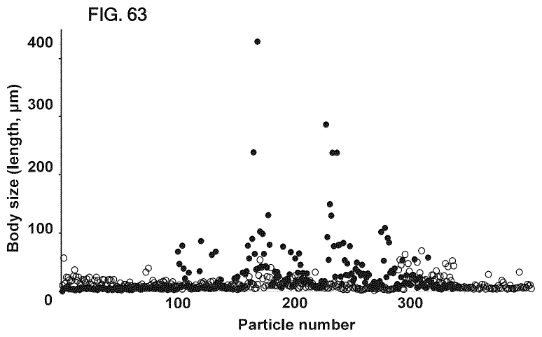

FIG. 63 shows a graphical representation of the enrichment of extracellular matrix bodies by filtration from a biological fluid, bovine vitreous humor, using a cellulose acetate membrane filter with a pore size of 0.22 micron filter. A greater abundance of extracellular matrix bodies were found in the isolate (filled circles) as compared to the filtrate (open circles).

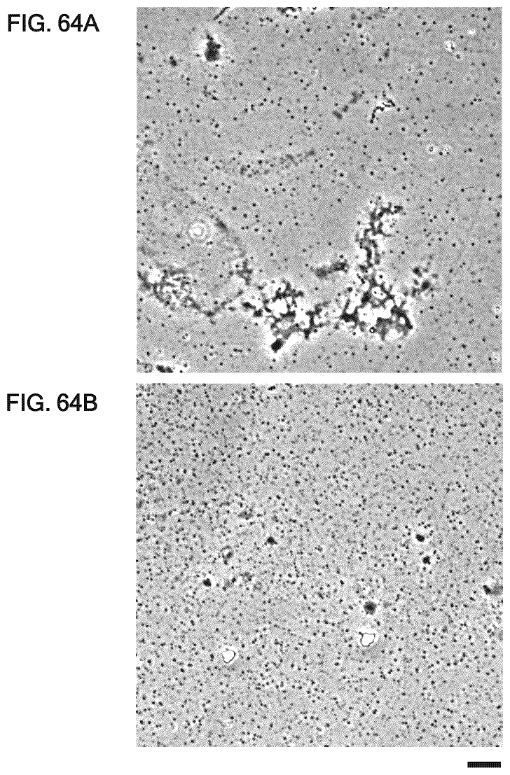

FIG. 64A shows a representative photomicrographic of the enrichment of extracellular matrix bodies by serial (repeated) filtration from a biological fluid, bovine vitreous humor, using a cellulose acetate membrane filter, first with a pore size of 0.22 micron, and second with a pore size of 0.45 micron. Extracellular matrix bodies were recovered by reversing the filter and extracting material from the proximal side of the filter to obtain an isolate. Image was taken on a glass slide with EDC fixation. Scale bars are 20 um. This image shows the double isolate being the isolate after filtration at 0.22, then 0.45 micron.

FIG. 64B shows a representative photomicrographic of the enrichment of extracellular matrix bodies by serial (repeated) filtration from a biological fluid, bovine vitreous humor, using a cellulose acetate membrane filter, first with a pore size of 0.22 micron, and second with a pore size of 0.45 micron. Extracellular matrix bodies were recovered by reversing the filter and extracting material from the proximal side of the filter to obtain an isolate. Image was taken on a glass slide with EDC fixation. Scale bars are 20 um. This image shows the filtrate after filtration at 0.22, then 0.45 micron. Relatively few extracellular matrix bodies, if any, were found in the double filtrate as compared to the double isolate.

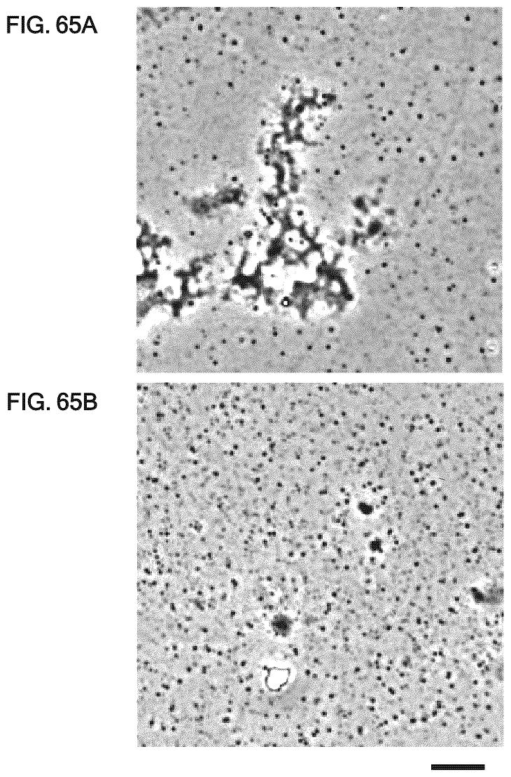

FIG. 65A shows an expansion of the micrograph in FIG. 64A.

FIG. 65B shows an expansion of the micrograph in FIG. 64B.

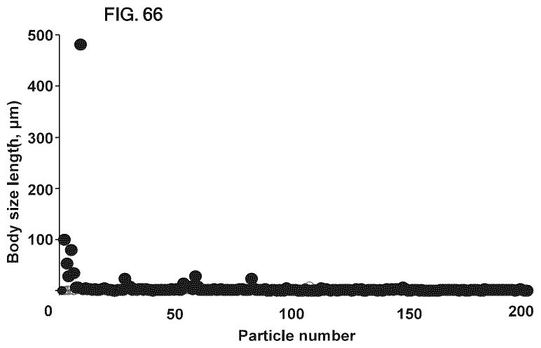

FIG. 66 shows a graphical representation of the enrichment of extracellular matrix bodies by serial filtration from a biological fluid, bovine vitreous humor, using a cellulose acetate membrane filter, first with a pore size of 0.22 micron, and second with a pore size of 0.45 micron. A greater abundance of extracellular matrix bodies were found in the double isolate (filled circles) as compared to the double filtrate (open circles).

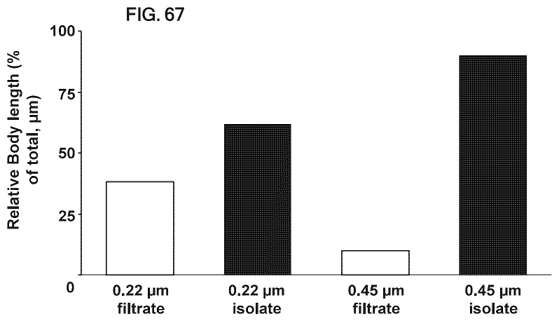

FIG. 67 shows a graphical representation of the enrichment of extracellular matrix bodies by serial filtration from a biological fluid, bovine vitreous humor, using a cellulose acetate membrane filter, first with a pore size of 0.22 micron, and second with a pore size of 0.45 micron. A greater abundance of extracellular matrix bodies were found in the isolate as compared to the filtrate in each step of the serial filtration.

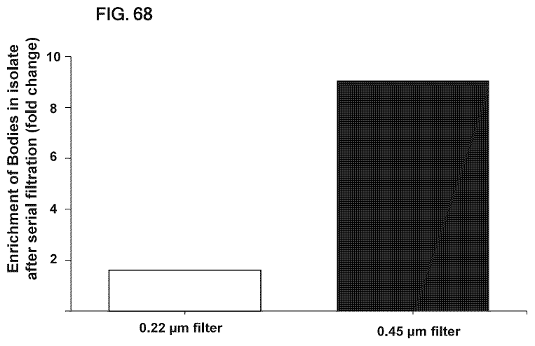

FIG. 68 shows a graphical representation of the enrichment of extracellular matrix bodies by serial filtration from a biological fluid, bovine vitreous humor, using a cellulose acetate membrane filter, first with a pore size of 0.22 micron, and second with a pore size of 0.45 micron. A multi-fold abundance of extracellular matrix bodies were found in the final isolate as compared to the final filtrate after serial filtration.

Example 24. Extracellular matrix bodies were isolated from human cerebrospinal fluid (CSF) by filtration. In this example, extracellular matrix bodies were isolated from human cerebrospinal fluid by membrane filtration.

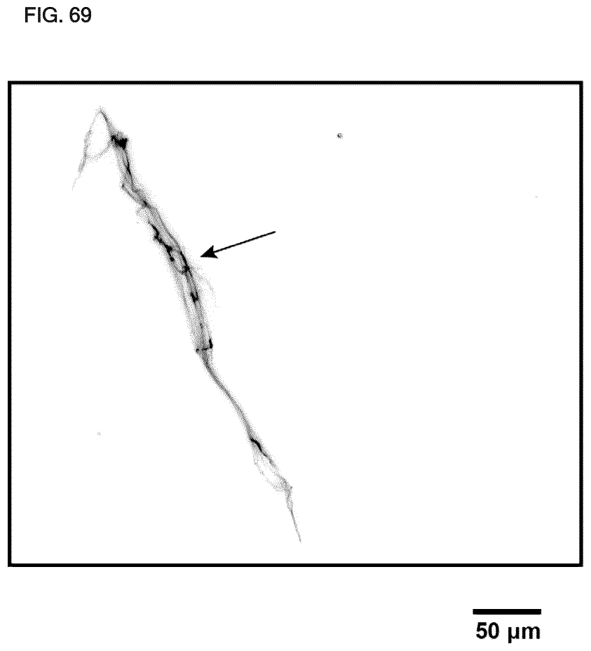

FIG. 69 shows a representative photomicrographic of extracellular matrix bodies isolated by membrane filtration from a biological fluid, human cerebrospinal fluid (CSF). CSF was filtered through a 0.45 μm syringe-tip filter. Extracellular matrix bodies were recovered from the proximal side of the membrane and fixed to a glass slide by an EDC-glutaraldehyde fixation protocol. The extracellular matrix bodies were stained with a monoclonal fluorescent-conjugated antibody (594) for fibronectin, an extracellular matrix protein. The slide was imaged by microscopy. Black lines and features in the image of FIG. 69 correspond to stained substances. This image shows that extracellular matrix bodies can be isolated from human cerebrospinal fluid (CSF) by the methods of this invention.

1-61. (canceled)

62. A process for separating, isolating or enriching extracellular matrix bodies in a biological fluid, the process comprising centrifuging or filtering the biological fluid.

63. The process of claim 62, comprising removing cells or cell debris by centrifuging the biological fluid; and separating, isolating or enriching extracellular matrix bodies from the biological fluid by centrifuging or filtering the biological fluid.

64. The process of claim 63, wherein the removing cells or cell debris by centrifuging the biological fluid comprises centrifugation which applies about 200 to 5,000 g for 1-10 minutes, or 100 to 1,200 g for 1-10 minutes.

65. The process of claim 62, wherein the separating, isolating or enriching extracellular matrix bodies from the biological fluid by centrifuging creates a pellet by applying forces for times and speeds below levels needed to force small particles into the pellet, so that the pellet is substantially free of particles smaller than about 1 micrometer.

66. The process of claim 62, wherein the separating, isolating or enriching extracellular matrix bodies from the biological fluid by centrifuging creates a pellet by applying 3,000-20,000 g for 1-100 minutes, or 6,000-100,000 g for 1-200 minutes, and optionally resuspending the pellet.

67. The process of claim 62, wherein the separating, isolating or enriching extracellular matrix bodies from the biological fluid by filtering is performed with a filter for passing particles of less than a cutoff size, wherein the cutoff size is 1, or 2, or 3, or 4, or 5, or 6, or 10 micrometers, and the extracellular matrix bodies are extracted from the filter and re-suspended.

68. The process of claim 62, comprising additional separating, isolating or enriching extracellular matrix bodies from a supernatant after centrifuging the biological fluid, or from a retentate, precipitate, residue, or extract after filtering the biological fluid, or from a filtrate after filtering the biological fluid, wherein the additional separating, isolating or enriching is done by microfluidic device, centrifugation, chromatography, chemical precipitation, filtration, immuno- or affinity-capture, electrophoresis, AC electrokinetics, or a combination thereof.

69. The process of claim 62, wherein the separating, isolating or enriching captures at least a majority of or substantially all of the extracellular matrix bodies from the biological fluid, optionally with an absence of cells.

70. The process of claim 62, wherein the separated, isolated or enriched extracellular matrix bodies comprise biomarkers in the form of a protein, a polypeptide, a lipid molecule, a lipoparticle, a carbohydrate, a nucleic acid molecule, or an expression level of a nucleic acid.

71. The process of claim 62 further comprising applying an initial centrifugation step to the biological fluid for removing cells or cell debris; and applying one or more serial centrifugation steps to the supernatant of the initial centrifugation, wherein a supernatant is used in each step.

72. The process of claim 71, wherein the removing cells or cell debris by centrifuging the biological fluid comprises centrifugation which applies about 200 to 5,000 g for 1-10 minutes, or 100 to 1,200 g for 1-10 minutes.

73. The process of claim 71, wherein the one or more serial centrifugation steps each apply about 1,000 to 60,000 g for 1-100 minutes, or 1,000 to 3,000 g for 1-30 minutes to the supernatant from the previous step.

74. The process of claim 71, comprising filtering the supernatant from a step of serial centrifugation with a filter for passing particles of less than a cutoff size; wherein the cutoff size is 1, or 2, or 3, or 4, or 5, or 6, or 10 micrometers, and extracting extracellular matrix bodies trapped in the filter.

75. The process of claim 71, comprising additional separating, isolating or enriching extracellular matrix bodies from a supernatant after centrifuging the biological fluid, wherein the additional separating, isolating or enriching is done by microfluidic device, centrifugation, chromatography, chemical precipitation, filtration, immuno- or affinity-capture, electrophoresis, AC electrokinetics, or a combination thereof.

76. The process of claim 71, wherein the separating, isolating or enriching captures at least a majority of or substantially all of the extracellular matrix bodies from the biological fluid, optionally with an absence of cells.

77. The process of claim 71, wherein the separated, isolated or enriched extracellular matrix bodies are a biomarker for medical, diagnostic or prognostic information.

78. The process of claim 71, wherein the separated, isolated or enriched extracellular matrix bodies comprise biomarkers in the form of a protein, a polypeptide, a lipid molecule, a lipoparticle, a carbohydrate, a nucleic acid molecule, or an expression level of a nucleic acid.

79. The process of claim 62, comprising separating, isolating or enriching extracellular matrix bodies by:
a. re-suspending a pellet obtained after centrifugation;
b. filtering the re-suspension with a filter for passing particles of less than a cutoff size, wherein the cutoff size is 1, or 2, or 3, or 4, or 5, or 6, or 10 micrometers; and
c. extracting extracellular matrix bodies trapped in the filter.

80. The process of claim 79, wherein the filtering captures at least a majority of or substantially all of the extracellular matrix bodies from the biological fluid, and the re-suspension is substantially free of particles smaller than about 1 micrometer.

81. The process of claim 79, comprising adding a reagent to a supernatant after the initial centrifugation, wherein the reagent is for precipitating the extracellular matrix bodies.

82. The process of claim 79, wherein the extracellular matrix bodies are at least 2-fold, or at least 5-fold, or at least 10-fold enriched in concentration as compared to the biological fluid.

83. The process of claim 79, wherein the extracellular matrix bodies are associated with a pathology or disease.

84. The process of claim 62, wherein the biological fluid is any one of whole blood, blood plasma, blood serum, cerebrospinal fluid, vitreous humor, aqueous humor, urine, saliva, sweat, tears, synovial fluid, pleural fluid, gastric fluid, peritoneal fluid, breast milk, nipple aspirate, ocular fluid, semen, amniotic fluid, lymph, bile, cerumen, chyle, chyme, endolymph, perilymph, exudates, feces, ejaculate, gastric acid, gastric juice, mucus, pericardial fluid, pus, rheum, sebum, serous fluid, smegma, sputum, synovial fluid, vaginal secretion, menstrual effluent, vomit and combinations thereof.

85. The process of claim 62, comprising determining a level of a biomarker of the separated, isolated or enriched extracellular matrix bodies, optionally wherein the biomarker is the level of the extracellular matrix bodies, or the level of a substance found in the extracellular matrix bodies, wherein the substance is a protein, a polypeptide, a lipid molecule, a lipoparticle, a carbohydrate, a nucleic acid molecule, or an expression level of a nucleic acid, optionally wherein the level of the substance is determined by any of microscopy, immunostaining, fluorescence assay, chelate complexation, quantitative HPLC, spectrophotometry, antibody array, Western blot, immunoassay, immunoprecipitation, ELISA, LC-MS, LC-MRM, radioimmunoassay, mass spectrometry, 2D gel mass spectrometry, LC-MS/MS, RT-PCR, nucleic acid assay, next generation sequencing, and combinations thereof.

86. A process for diagnosing, prognosing or monitoring a disease in a subject, the process comprising:
a) separating, isolating or enriching extracellular matrix bodies in a biological fluid sample of the subject;
b) determining a level of one or more biomarkers based on the separated, isolated or enriched extracellular matrix bodies, wherein the biomarker is the level of the extracellular matrix bodies, or the level of a substance found in the extracellular matrix bodies, wherein the substance is a protein, a polypeptide, a lipid molecule, a lipoparticle, a carbohydrate, a nucleic acid molecule, or an expression level of a nucleic acid; and
c) comparing the levels of the biomarkers to reference levels based on a control group of subjects, and diagnosing, prognosing or monitoring the disease in the subject.

87. A method for preparing a sample of extracellular matrix bodies in a biological fluid by fixation, the method comprising contacting the biological fluid with a non-reversible cross-linking agent which fixes the extracellular matrix bodies, optionally wherein the non-reversible cross-linking agent is a water-soluble carbodiimide, a cyanogen halide, or a mixture thereof, optionally selected from the group consisting of 1-ethyl-3-(3-dimethylaminopropyl)-carbodiimide, cyanogen bromide, cyanogen fluoride, cyanogen chloride, or cyanogen iodide.

88. The method of claim 87, comprising contacting the biological fluid with an aldehyde-containing fixative agent.

89. The method of claim 87, comprising detecting the fixed extracellular matrix bodies by microscopy, spectrophotometry, tomography, or magnetic resonance.
